(19) United States
(12) Patent Application Publication
(10) Pub. No.: US 2021/0285017 A1
(43) Pub. Date: Sep. 16, 2021

(54) METHOD AND SYSTEM FOR IMPROVING THE GREENHOUSE GAS EMISSION REDUCTION PERFORMANCE OF BIOGENIC FUELS, HEATING MEDIUMS AND COMBUSTION MATERIALS AND/OR FOR ENRICHING AGRICULTURAL AREAS WITH CARBON-CONTAINING HUMUS

(71) Applicants: Marc FELDMANN, Berlin (DE); Lennart FELDMANN, Marburg (DE)

(72) Inventors: Marc Feldmann, Berlin (DE); Lennart Feldmann, Marburg (DE); Michael Ferldmann, Marburg (DE)

(21) Appl. No.: 16/339,596

(22) PCT Filed: Oct. 6, 2017
(86) PCT No.: PCT/EP2017/075516

§ 371 Date: Apr. 4, 2019

(30) Foreign Application Priority Data
Oct. 7, 2016 (DE) 10 2016 011 952.7
Jun. 14, 2017 (DE) 10 2017 005 627.7

(51) Int. Cl. C12P 5/02; C10L 3/08; C10L 5/44; C10L 9/08; C12M 1/107; C05F 11/02; C10B 53/02; C10B 57/14

(52) U.S. Cl. CPC C12P 5/023; C10L 2290/542; C10L 5/447; C10L 9/08; C10L 9/083; C10L 9/086; C12M 21/04; C05F 11/02; C10B 53/02; C10B 57/14; C10L 2200/0469; C10L 2230/02; C10L 2200/0446; C10L 2270/026; C10L 2200/0423; C10L 2270/023; C10L 2290/28; C10L 2290/24; C10L 2290/06; C10L 2230/14; C10L 2230/22; C10L 2290/02; C10L 2290/04; C10L 2290/08; C10L 2290/10; C10L 2290/18; C10L 2290/22; C10L 2290/26; C10L 2290/30; C10L 2290/42; C10L 3/08

(57) ABSTRACT

A method and a system for improving the GHG emission reduction performance of fuels, heating mediums and combustion materials and for enriching agricultural land with C-containing humus.

### 1. TECHNICAL FIELD

The invention relates to the production of greenhouse gas (GHG) emission-reduced fuels, heating mediums and combustion materials from biomass and to the ensuring or improvement of the quality of agricultural and forest areas by ensuring or improving the humus content thereof.

### 2 BACKGROUND

Transport is the basis of our society and economy. Mobility is the lifeblood of the European internal market, it shapes the quality of life of the citizens who enjoy their freedom to travel. Efficient mobility is also a prerequisite for economic prosperity. No transport is no option, free travel, goods transport and trade are and will remain basic needs for the people. However, given the limited resources and in the light of greenhouse gas emissions (GHG emissions) caused by transport, society, politicians and players in the mobility industry are called upon to meet the travel needs of the citizens and the freight traffic needs of our economy in a new way.

As early as in 1988, the former NASA scientist, Dr. James Hansen, was the first to describe the greenhouse gas effect, warned against it and received little attention. Today, almost 30 years later, in a study published in March 2016, he and his team warn that the climate models that have been drawn up do not property reflect but trivialize the speed of global warming and climate change. Even with a global warming of ‘only’ 2° C., the imminent rise of sea levels by a few meters would endanger the existence of entire regions and countries.

At the same time, oil will become scarcer in the coming decades and increasingly come from uncertain sources. In the long term, the less successfully the world masters the transition to non-fossil energy sources, the higher the rise in oil prices will be. If we do not manage this dependency on oil, it might have a drastic impact on travel and goods transport, with serious consequences for price stability, trade, employment and thus for our economic security and social peace.

In Paris in December 2015, the international community of states decided to limit the increase in the mean temperature of the atmosphere of the earth compared with pre-industrial levels to below 2° C. and thus the climate change. In order to achieve this global political goal, the EU must reduce emissions of European greenhouse gases by 80% to 95% by 2050 compared with 1990 levels. It has not yet been decided by politicians whether the target value of 80% or 95% will ultimately have to be reached.

According to the statistics of the German Federal Environment Agency, the German share in European GHG emissions amounted to about 1.251 million tons of CO₂ equivalents in 1990, so that Germany has an emission target of a maximum of 63-250 million tons of CO₂-eq for the year 2050. This target was also confirmed by the German government in its 2010 energy concept. It was reaffirmed in 2014 in the “Klimaschutz 2020 [Climate Protection 2020]” action program of Federal Government and in 2016 laid down in the “Klimaschutzplan 2050 [Climate Protection Plan 2050]” and backed up with measures. As interim emission targets, the German policy program specifies a residual GHG emission of a maximum of 751 million tons for 2020 (down 40% compared to 1990 levels), a reduction of a maximum of 563 million tons (down 55%) for 2030 and a GHG emission reduced to a maximum of 375 million tons (down 70%) for 2040.

By 2010, German GHG emissions had decreased from 1.251 million t CO₂-eq in 1990 to 941 million t CO₂-eq in 2010. (−25%), in 2015 it amounted to only 902 million tons CO₂-eq (−28%). In 2016, however, it had already reached 906 million tons of CO₂-eq (−27.6%) again. If the first interim target is to be reached in 2020, GHG emissions must be reduced by 38.75 million t CO₂-eq (each −3.1 percentage points/a compared to 1990) in each of the 4 years remaining until 2020 and thereafter by 18.80 million t CO₂-eq/year annually (each −1.5 percentage points/a compared to 1990). It is already foreseeable that the measures adopted so far will not be sufficient to achieve the GHG emission reduction performance targets.

In the transport sector where the reduction in the GHG emissions is particularly difficult and therefore particularly costly due to a lack of alternatives, the emission reduction should reach by 2050 at least 60% compared to the 1990 levels. According to the statistics of the German Federal Environment Agency, which leaves GHG emissions arising in other sectors where they are and thus does not apply the Life Cycle Analysis (LCA) method developed by the IPCC (Intergovernmental Panel on Climate Change) and the UNFCCC (United Nations Framework Convention on Climate Change), the share of German traffic in German GHG emissions in 1990 was about 164 million t CO₂-eq, which corresponds to 13.1% of the total GHG emissions. By 2010, the GHG emission level had fallen to 154 million t CO₂-eq, which is less than the overall development, but the share of traffic had risen to 16.4% of the total emissions. By 2015, the GHG emissions from German traffic had increased both in absolute and relative terms, namely to 161 million t CO₂-eq, and has a share of 17.8% in Germany's total GHG emissions. While the total German GHG emissions fell by 28% in the 25 years from 1990 to 2015, the GHG emissions from German traffic fell by only 3 million t CO₂-eq, or by 1.8%, from 164 million t CO₂-eq to 161 million t CO₂-eq during this period. This shows how difficult it is to achieve GHG emission reductions in the transport sector.

Nevertheless, the study “Klimaschutzbeitrag des Verkehrs bis 2050 [Contribution of transport to climate protection by 2050]” commissioned by the German Federal Environment Agency in June 2016 proposes to set the GHG emission reduction targets for German transport (excluding international transport) for 2020 at 15%-20%, for 2030 at 25%-40%, for 2040 at 43%-70% and for 2050 at 60%-98% compared to 1990. GHG emissions from transport, excluding GHG emissions from upstream production chains, will thus only be allowed to reach 131-139 million t CO₂-eq in 2020 (−10% to −15% compared to 2005), only 98-123 million t CO₂-eq in 2030 (−20% to −36% compared to 2005), only 49-93 million t CO₂-eq in 2040 (−40% to −68% compared to 2005) and only 3-66 million t CO₂-eq in 2050 (−57% to −98% compared to 2005).

According to this study, the GHG emissions from transport should mainly be reduced by reducing the energy consumption, namely by 12-16% by 2020 compared with the energy consumption of 2005, by 21-31% by 2030 compared with 2005, by 31-45% by 2040 and by 40-60% by 2050. This means that by 2020 fuels will have virtually no GHG emission reduction potential, as fuel consumption is 12%-16%, which is even higher than the GHG reduction target of 10%-15%. By 2030, 86% to 105% of the GHG reduction performance shall be achieved via the fuel consumption reduction and 14% to −5% via the specific GHG emission reduction related to an energy unit (the fuel reduction consumption of 21% to 31% accounts for about 105% to 86% of the GHG reduction of 20% to 36%). By 2040, 66%-78% of the GHG emission reduction performance should still be achieved via the fuel consumption reduction and only 34%-22% via the specific GHG emission reduction related to an energy unit (the reduction in fuel consumption of 31%-45% accounts for about 78%-66% of the GHG reduction of 40%-68%). And in 2050, 61%-70% of the GHG reduction performance shall be achieved via the fuel consumption reduction and only 39% to 30% via the specific GHG emission reduction related to an energy unit (the fuel consumption reduction of 40% to 60% accounts for about 70% to 61% of the GHG reduction of 57%-98%). The study aims to achieve the GHG reduction performance mainly by producing liquid and gaseous fuels from renewable electric current (Power to Liquid PtL and Power to Gas PtG), with the GHG emissions associated with electricity production being attributed to the energy sector rather than to transport—in the form of deceptive packaging, so to speak.

The study “Klimaschutzbeitrag des Verkehrs bis 2050”, which was carried out in June 2016 and is the latest study to be produced by German transport experts as part of the Mobility and Fuel Strategy (MFS) by the German Government, which is currently being developed, shows that the contribution of biofuels to reducing the GHG emissions is estimated to be low. Therefore, only a total biofuel quantity of only 300 PJ/a (83,333 GWh_{Hi}/a) is used in the plans, the production of which in the upstream chain that is not considered by the study will still cause GHG emissions of 10 million t CO₂-eq in 2050, which corresponds to 120 gCO₂/kWh_{Hi}.

By far the largest share of GHG emissions from all German transport (air, water, rail and road transport, excluding the German share in international maritime transport) is accounted for by road transport, with about 94%-96%. In 1990, its share of the total German GHG emissions amounted to about 154 million t CO₂-eq (12.3%), in 2010 to about 148 million t CO₂-eq (15.7%) and in 2015 to about 155 million t CO₂-eq (17.1%). It is therefore mainly road traffic that is to blame for the fact that the relative emissions of German traffic have risen from 13.1% in 190 to 17.8% in 2015.

These GHG values from the German Federal Environment Agency are still about 20% too low since as presented above they are not based on the Life Cycle Analysis method (LCA), which is also referred to as Well-to-Tank analysis and includes the GHG emissions from the entire fuel production path. Only the direct combustion-related (stoichiometric) greenhouse gas emissions that are determined in the tank-to-wheel analysis are taken into account. In addition, unrealistic consumption values from vehicle registration (keyword: New European Driving Cycle (NEDC)) and no so-called “real driving” values are used for national climate reporting. In order to achieve, within the framework of national climate reporting, reliable GHG emission values that correspond to the actual national fuel consumption, considerable corrective calculations must be made. GHG emissions caused by the production of fuels are therefore not included in the greenhouse gas balance of transport any more than GHG emissions caused in the production of electricity used as fuel. In national climate reporting, these GHG emissions are allocated to the other sectors. This means that effects of climate protection measures affecting the GHG emissions from electricity and fuels used in transport are only of limited significance.

The fact that emission values determined on the test bench have very little to do with the RDE (real driving emissions) occurring during real driving operations is demonstrated not least by the diesel exhaust scandal. Even after the engine control programs had been improved, the RDE (real driving emissions) for nitrogen oxides (NOₓ) exceed the test bench values determined in accordance with NEDC by a factor of 3-5. This flagrant violation was classified as permissible by at least one of the world's leading automobile manufacturers and the German Federal Motor Transport Authority (Kraftfahrt-Bundesamt) contrary to the applicable environmental legislation, which shows how difficult it is for the automotive industry to comply with the emission values stipulated by European legislation.

In the following, the adjusted LCA-GHG emissions of the fuels used in German road traffic including electricity (in 2015 about 636,000 GWh or about 2,290 PJ), determined according to the Life Cycle Analysis (LCA) and in the Real Driving Mode, will therefore be considered, which did not lead to a GHG emission of 155 million t CO₂-eq in 2015 but to a GHG emission of about 186 million t CO₂-eq.

Despite considerable technical progress, the European transport system has not changed fundamentally. It is not sustainable. Although transport has become more energy-efficient, it is still about 95% dependent on oil and oil-based fuels in the EU. Transport has also become more environmentally friendly, but an increase in traffic has more than compensated for this positive development. New propulsion technologies, new fuels and better traffic management are therefore needed, in both the EU and the rest of the world, in order to reduce transport emissions of greenhouse gases, nitrogen oxides, fine particulates and noise. Delay and a hesitant introduction of new technologies should not be weighted by the missing effect of the first years, but by the missing effects of the target years 2020 or 2030 or 2040 or 2050, which are much greater.

The challenge, therefore, is to eliminate dependence of transport on oil without sacrificing its efficiency and reducing mobility. At the same time, existing resources must be used sustainably to provide high-quality mobility options. In practice, transport must consume more environmentally friendly non-fossil energy, make better use of modern infrastructure and reduce its negative impact on the environment and important natural assets, such as water, land and ecosystems.

The car and its use must therefore be reinvented. We must act without hesitation. The decisions we make today are crucial for transport in 2050. By 2020, 2030 and 2040, ambitious interim targets must be reached to ensure that we move in the right direction.

This includes the development and introduction of sustainable and environmentally friendly fuels and propulsion systems. At present, we are still dependent on the oil-based fossil fuels, i.e. gasoline and diesel, but the future belongs to advanced alternative fuels with high and very high emission reduction performance. However, according to the latest EU directives, biofuels produced from food or feed are not advanced fuels because they do not comply with the ethical component of the sustainability principle.

The European Council, the European Commission, the European Parliament and the Member States have therefore set themselves the target of reducing current greenhouse gas emissions from the transport sector (see Directive 2015/1513 of the European Parliament and of the Council of 9 Sep. 2015). By 31 Dec. 2020, fossil fuel suppliers must reduce their life cycle greenhouse gas emissions (LCA-GHG emissions) per unit energy by at least 6%. In addition, research and development on new advanced biofuels shall be promoted, which will allow high greenhouse gas emission savings and will not compete directly for agricultural land for food and feed production.

The EU (Council, Commission, Parliament, Member States) considers it desirable to achieve a significantly higher consumption of advanced fuels by 2020 compared to the current amount consumed since they will play an important role in reducing CO₂ emissions from transport and in developing low CO₂ emission transport technologies, especially after 2020. In particular, preference shall be given to biogenic input materials that do not have a high economic value for uses other than the production of biofuels and that have high greenhouse gas emission reduction performance. In this context, waste and residual materials are of particular importance as potential input materials for fuel production. These EU targets, which were published in September 2015, thus contradict the more recent targets of the German study “Klimaschutzbeitrag des Verkehrs bis 2050” published by the German Federal Environment Agency in June 2016.

Since the GHG emission reduction performance of fuels produced from waste and residual materials is particularly high, the EU, for example, will credit these with twice their energy content to the respective national target to be achieved by the member states, namely to achieve a biofuel share of at least 10% of the total fuels consumed in the transport sector by 31 Dec. 2020. The main objective of all efforts is therefore to reduce current GHG emissions. This is often forgotten, e.g. when considering and evaluating the sub-goal of energy efficiency.

The atmosphere of the earth contains a certain proportion of greenhouse gases (GHG), including water vapor, carbon dioxide (CO₂, also known as carbon dioxide for short), methane (CH₄) and laughing gas (dinitrogen monooxide N₂O, also known as nitrogen oxide for short). The quantities of greenhouse gases contained in the atmosphere of the earth and their changes are measured in millions or billions of tons. According to the new EU Directive 2015/652 of the EU Council of 20 Apr. 2015, laughing gas is about 298 times more harmful to the environment than carbon dioxide and methane is about 25 times more harmful to the environment. In this context, environmental harmfulness is equated with the temperature-increasing effect of greenhouse gases on the atmosphere of the earth.

In order to standardize the various environmental impacts, experts use the environmental impact of the greenhouse gas, i.e. carbon dioxide, as a reference value. The absolute total level of the various greenhouse gases in the atmosphere of the earth and its change are correspondingly measured and reported in million or billion tons of CO₂ equivalents (CO₂-eq). The relative CO₂ level is measured and indicated as the relative share value “part per million” (ppm). The relative proportion of CO₂ in the pre-industrial era was about 300 ppm and is today about 400 ppm. The further increase until the year 2100 shall be limited to 550 ppm.

Every combustion process in which fossil carbon (C) oxidizes with atmospheric oxygen (O₂) to give CO₂ increases the CO₂ level in the atmosphere of the earth (measured in million or billion tons of CO₂ equivalents). Accordingly, any anaerobic fermentation process in which fossil or atmospheric carbon (C) combines with hydrogen (H) to form methane (CH₄)—as is the case, for example, in rice paddies and the stomachs of every cattle—also increases the level of CO₂ equivalents in the atmosphere of the earth. Likewise, each nitrification and denitrification process that occurs during fertilization, during which laughing gas (N₂O) is formed, increases the level of CO₂ equivalents in the atmosphere of the earth. These processes are all chemical and therefore technical processes.

Accordingly, the amount of greenhouse gas emissions of a fuel, heating medium or combustion material related to an energy unit and measured in gCO₂ equivalents/MJ or in gCO₂ equivalents/kWh is a technical value. It does not matter whether this technical value of the greenhouse gas emission into the atmosphere of the earth (greenhouse gas pollution for short) results from the chemical process of (stoichiometric) combustion or according to the Life Cycle Analysis (LCA), which also considers processes that are upstream and possibly downstream of stoichiometric combustion. The LCA consideration is also referred to as Well-to-Wheel analysis, which consists of the two sections Well-to-Tank and Tank-to-Wheel, wherein the Tank-to-Wheel section covers stoichiometric fuel combustion in the engine.

In this context, a distinction must be made between a reduction in the GHG emission rate and a reduction in greenhouse gases (GHG). A reduction in the GHG emission (rate) merely means that the emission of GHG (the electricity of additional GHG) decreases, a reduction in greenhouse gases, on the other hand, reduces the absolute stock of GHG quantities contained in the atmosphere of the earth. Accordingly, the UNFCCC and Kyoto Protocol bodies also distinguish between emission reduction units (ERUs) and credits from GHG or carbon reductions (removal units RMUs) in accordance with the Annex to Decision 13/CMP.1.

In disagreement with the TREMOD model of the Federal Environment Agency and in agreement with all national and international environmental authorities (e.g. the Intergovernmental Panel on Climate Change (IPCC), the United Nations Framework Convention on Climate Change (UNFCCC)), government agencies, such as the Ministry of Finance, and supranational agencies, such as the EU Commission, the European Parliament and the European Council, the GHG emissions of the various energy sources are considered in the following over their entire development process, i.e. on the basis of a so-called Life Cycle Analysis (LCA) or Well-to-Wheel (WtW).

As a reference value for the greenhouse gas load of a fuel, heating medium or combustion material, experts take the life cycle greenhouse gas emissions resulting from the production, distribution and use of the (fossil) fuels, i.e. gasoline (fuel for Otto engines) and diesel (diesel fuel), produced from fossil crude oil over the entire production and use path. The greenhouse gas emission (or also greenhouse gas intensity, greenhouse gas load, greenhouse gas balance) of this fossil reference determined according to LCA has so far been 83.8 gCO₂-eq/MJ, corresponding to 301.7 gCO₂-eq/kWh_{Hi}, in accordance with EU Directive 2009/28/EC.

The EU Directive 2015/652 of the EU Council, dated 20 Apr. 2015, for determining the calculation methods regarding the quality of gasoline and diesel fuels redefines in detail the method for calculating the greenhouse gas intensity and the corresponding standard values. In order to avoid repetitions, reference is made to this EU Directive with regard to the calculation of GHG emission values (greenhouse gas intensity) and with regard to the absolute GHG emission values, in particular also to the “Well-to-Tank report” (version 4) of July 2013 of the Joint Research Center-EUCAR-CONCAWE (JEC Consortium) on which this Directive is based.

According to the weighted average of the respective sources of raw materials, the standard values for fossil fuels, which are detected or determined according to the LCA, are now 93.3 gCO₂-eq/MJ (335.9 gCO₂-eq/kWh_{Hi}) for fuel for Otto engines, 95.1 gCO₂-eq/MJ (342.4 gCO₂-eq/kWh_{Hi}) for diesel fuel, 73.6 gCO₂-eq/MJ (265.0 gCO₂-eq/kWh_{Hi}) for liquid gas, 69.3 gCO₂-eq/MJ (249.5 gCO₂-eq/kWh_{Hi}) for compressed natural gas (CNG), 74.5 gCO₂-eq/MJ (268.2 gCO₂-eq/kWh_{Hi}) for liquefied natural gas (LNG), 3.3 gCO₂-eq/MJ (11.9 gCO₂-eq/kWh_{Hi}) for compressed synthetic methane (syn-methane) produced according to Sabatier, 104.3 gCO₂-eq/MJ (375.5 gCO₂-eq/kWh_{Hi}) for compressed hydrogen from steam reformed natural gas, 9.1 gCO₂-eq/MJ (32.8 gCO₂-eq/kWh_{Hi}) for compressed hydrogen from electrolysis run with green electricity, 234.4 gCO₂-eq/MJ (843.8 gCO₂-eq/kWh_{Hi}) for compressed hydrogen from coal, 52.7 gCO₂-eq/MJ (189.7 gCO₂-eq/kWh_{Hi}) for compressed hydrogen from coal with CO₂ capture, and 86 gCO₂-eq/MJ (309.6 gCO₂-eq/kWh_{Hi}) for fuel for Otto engines or diesel fuel from fossil used plastics. According to the EU Directive 2015/652, the weighted average of all fossil fuels, referred to as the fuel base value, is now 94.1 gCO₂-eq/MJ (338.8 gCO₂-eq/kWh_{Hi}), i.e. 10.3 gCO₂-eq/MJ (37.1 gCO₂-eq/kWh_{Hi}) more than before.

A reduction of this (technical) reference value as specified by the EU Commission by 1.00% corresponds to a reduction in the LCA-GHG emission by 3.388 gCO₂-eq/kWh_{Hi}. This reduction in the LCA greenhouse gas emission value and each multiple thereof as well as the initial value (fossil reference value) represent technical values because the reduction in a (generally recognized) technical value is the (technical) result of a technical process. Accordingly, the terms “greenhouse gas emission savings”, “GHG emission reduction” and “GHG emission reduction performance” also describe technical issues.

The paper by Katja Kolimuss: “Carbon offsets 101”, World watch Magazine, vol. 20, no. 4, Washington July 2007 (www.worldwatch.org/node/5134) and Unknown: “Understanding carbon offsets”, Offset Options S:L:, Barcelona, 2010 (www.offsetoptions.com/faq_carbonoffsets.php) represent the background of the subject area. But even these sources mistakenly equate GHG reductions with the reduction in GHG emissions. While GHG reductions actually lead to a lower GHG level in the atmosphere of the earth, GHG emission reductions only result in a reduction in the GHG emission rate; in the latter case, however, further GHG emissions occur, so that the GHG level of the atmosphere of the earth continues to increase—although at a slower pace. Unfortunately, these two points of view are often confused—even at the technical level. The result is wrong conclusions.

It is essential for the classification and assessment of the method according to the invention and the system according to the invention to distinguish these technical terms. In this context, avoided GHG emissions (i.e. the reduction in the emission rate) cannot be equated with CO₂-binding measures. The former reduce the emission rate of fossil carbon, the latter, such as reforestation, actually remove atmospheric carbon from the atmosphere of the earth via photosynthesis, although not really permanently, but only over the lifetime of the respective tree or wood product (i.e. for about 20 to 500 years). The tree or wood product then decays, the aerobic decay being a chemical oxidation process that uses atmospheric oxygen, so that the atmospheric carbon ultimately ends up back in the atmosphere of the earth.

The EU Directive 2009/28/EC (RED 1) reports for a large number of biogenic and synthetic fuels and their various production methods how high they are loaded with GHG emissions. The emission values were determined using the LCA method. None of the biofuels listed in the directive and also none of the synthetic fuels listed have GHG emissions of 0 gCO₂-eq/MJ or 0 gCO₂-eq/kWh_{Hi}. The best values are achieved with 10 gCO₂-eq/MJ (36 gCO₂-eq/kWh_{Hi}) of biodiesel from vegetable or animal waste oil and with 12 gCO₂-eq/MJ (43.2 gCO₂-eq/kWh_{Hi}) of biogas produced from dry manure. From the so-called future fuels, synthetic diesel produced from waste wood according to the Fischer-Tropsch process reaches 4 gCO₂-eq/MJ (14.4 gCO₂-eq/kWh_{Hi}), DME produced from waste wood and methanol produced from waste wood reach 5 gCO₂-eq/MJ (18 gCO₂-eq/kWh_{Hi}), Fischer-Tropsch diesel produced from farmed wood reaches 6 gCO₂-eq/MJ (21.6 gCO₂-eq/kWh_{Hi}) and DME produced from farmed wood and methanol produced from farmed wood reach 7 gCO₂-eq/MJ (25.2 gCO₂-eq/kWh_{Hi}). Ethanol produced from wheat straw (ligno-ethanol) still reaches 11 gCO₂-eq/MJ (39.6 gCO₂-eq/kWh_{Hi}).

Therefore, even biofuels used as substitutes for fossil fuels in internal combustion engines and future synthetic fuels are not per se GHG-emission-free or GHG-neutral energy carriers; on the contrary, they can be significantly loaded with GHG emissions. According to the LCA method, the greenhouse gas load results from the sum of the (indirect) GHG loads of all energies or all energy carriers used in cultivation, harvesting, biomass storage, transport, conversion into marketable energy carriers, energy carrier storage, distribution and use as well as the (direct) GHG emissions which are emitted into the atmosphere of the earth in the form of N₂O, CH₄, CO₂ and other greenhouse gases.

Although the GHG load of transport is substantially reduced through the use of conventional biofuels and even more so through the use of synthetic fuels (according to the Evaluation and Experience Report 2015 of the German Federal Institute for Agriculture and Food (FAF), on the average by 70% in 2015), in practice to date none of the fuels listed in EU Directive 2009/28/EC has been able to achieve absolute GHG neutrality, i.e. GHG freedom (both of which correspond to a GHG emission reduction performance of 100%). This means that in practice there is still no fuel that can be used in internal combustion engines with a GHG emission of 0.0 gCO₂-eq/MJ or 0.0 gCO₂-eq/kWh_{Hi}, nor is there any absolutely GHG-free biofuel.

Synthetic methane (syn-methane) generated from grid-decoupled wind power and atmospheric CO₂ comes closest to the goal of GHG neutrality according to Sabatier. The study “Klimaschutzbeitrag des Verkehrs bis 2050” commissioned by the German Federal Environment Agency and published in June 2016 comes to the following conclusion (cf. page 95 and page 97): “Internal combustion engines can be operated virtually GHG-neutral via the life cycle of the fuel if the fuels are produced from renewable or RES electricity and CO₂ is circulated through the atmosphere” and “If a significant reduction in the GHG reductions shall be achieved, the use of energy carriers generated from renewable electricity Is necessary”. Other GHG-neutral fuels than so-called PtG and PtL fuels are obviously not known to the authors of the study, i.e. proven transport experts with above-average know-how, because at the same time the study published by the Federal Environment Agency in June 2016 comes to the following conclusion (see page 82): “Imported biofuels pose a particular challenge because they are not taken into account in the national inventory reports [Federal Environment Agency, 2014b], but unlike electricity or electricity-generated fuels they cannot be produced in a greenhouse gas-neutral fashion in the future either”. This statement fits in with the assumption made in the study that biofuels will still have GHG emissions of 120 gCO₂-eq in 2050 (see above).

In their assessment, the proven transport experts do not take into account the fact that the production costs of synthetic methane produced from wind power, in particular, are very high due to the low technical availability of the plant (wind blows only 2,000 to 3,000 hours in the 8,760 hours per year) and the associated high plant costs as well as due to the low efficiency of only 40% in total (70% in the case of electrolysis, 80% in the Sabatier process and 70% in the case of the sum of all other upstream and downstream processes, such as CO₂ extraction from the air and compression of the gas produced) and the associated high energy costs per kilowatt hour of gas fuel produced (9.2 cents/kWhₑₗ wind power/0.4=23 cents/kWh_{Hi} syn-methane). The energy costs alone amount to 23 cents/kWh_{Hi}×8.8 kWh_{Hi}/liter×1.19=241 cents/liter gasoline equivalent including VAT per liter gasoline equivalent. Plant and capital costs for the production plants, personnel costs, transport costs in the natural gas network, gas station costs as well as energy and value added taxes are not considered here yet at all.

Also the frequently mentioned restriction to wind power not discharged into the electricity grid (and therefore quasi free of charge) does not represent a solution because in this case the wind turbines are partly connected to the electricity grid, which means that the precondition set by the EU Commission for not taking into account the GHG load of the electricity mix—the grid decoupling of the renewable electricity generation plants—is no longer fulfilled. In addition, the technical availability of the electrolysis and synthesis plants goes back even further, to only the time when the electricity transport network is overloaded. Even if this share were set extremely high at 25% of the time the wind turbines run at all (2,000-3,000 h/a)—i.e. 500 to 750 hours per year—the very capita-intensive electrolysis and synthesis plants would remain unused for 8,010 to 8,260 hours per year. The calculation for syn-methane generated from wind power is thus ruined by the high costs of the grid-decoupled energy carriers used (renewable electricity) and/or by a low technical availability of the production facilities.

According to the “Klimaschutzbeitrag des Verkehrs bis 2050” study, the preferred technologies of the future for passenger cars, light-weight commercial vehicles and small trucks are battery-electric (BEV) and plug-in hybrid (PHEV) vehicles (cf. page 105). These vehicles are said to be entering the market increasingly after 2015 and will allegedly dominate registrations after 2050. In addition, niches for fuel cell vehicles are also taken into account. However, internal combustion engines with liquid fuels continue to dominate the vehicle stock. Gas vehicles (CNG and LPG) also remain a niche for heavy trucks and buses. The low-GHG energy supply for transport will be realized from 2030 onwards by converting fossil liquid fuels to Power-to-Gas (PtG) and/or Power-to-Liquid (PtL) fuels produced from renewable (RE) sources (cf. page 106). For the authors of the study vehicles equipped with internal combustion engines (CNG and LNG) are hardly worth mentioning, especially gas vehicles not powered by bio-methane.

Of the residual materials with high GHG emission reduction performance, those that are particularly suitable for the production of fuels are those that are available in large quantities. The straw growth is such a residual material available in large quantities which has a high GHG emission reduction performance. In Germany alone, about 44 million tons are produced annually with a (lower) calorific value Hi of about 180,000 GWh_{Hi} (650 PJ), of which about 29.8 million tons are grain straw (wheat straw, rye straw, barley straw, triticale straw and oat straw), about 9.9 million tons are rapeseed straw and about 4.0 million tons are grain corn straw.

However, this growth of straw cannot be fully used. In agricultural practice, even with “complete” straw removal, about 28% or about 12.2 million tons of wet mass of the national straw growth in the form of stubbles and chaff remain in the field, i.e. a maximum of 72% can be driven off at all. This means that only a maximum of 31.5 million tons of straw wet mass can be used at all. Since there is now a consensus among agricultural experts that an average of about ⅔ of the straw growth must remain in the fields in order to maintain the humus content of the arable land, especially to compensate for the effects which “soil predators”, such as corn, potatoes and sugar beets, have on the arable land, in Germany not only 12.2 million tons of the annual straw growth must remain in the fields, but about 29 million tons. This reduces the amount of straw that can be removed from German fields from about 44 million tons/a to about 15 million tons/a (the amount of 4.15 million tons/a needed as bedding for livestock farming does not count in ⅓ but in ⅔ because it is assumed that this straw returns to the field in the form of solid manure and thus contributes to maintaining the humus content). The proven experts of the German Biomass Research Center (GBRC) even only have a quantity of 8-13 million tons of straw wet mass per year available for energy purposes, which corresponds to about ⅕ to ⅓ of the national growth of straw.

The production of fuel from straw is such an energetic purpose. Assuming a conversion efficiency of 40%, a national straw removal of 8-15 million tons with a calorific value of about 33,000-61,000 GWh_{Hi} (120-220 PJ) can produce a fuel quantity of about 13,100-24,500 GWh_{Hi} (48-88 PJ). With a conversion efficiency increased to 70%, the amount of fuel that can be produced from German straw increases accordingly to 23,000-43,000 GWh_{Hi}(83-155 PJ).

46%-48% of the dry straw mass removed from the field consists of carbon. The cereal plant is known to obtain this carbon through photosynthesis from the carbon dioxide (CO₂) contained in the atmosphere. The carbon contained in the dry substance of the plant is thus of atmospheric origin. When this atmospheric carbon is removed (sequestered) from the atmosphere of the earth, the atmosphere of the earth is decarbonized.

Straw, which in Germany largely remains on the field after harvesting the cereal grains and is worked into the soil to maintain the humus content of the soil, is broken down into its components there. The straw is broken down by soil animals, microorganisms and fungi as well as by the physico-chemical process of aerobic rotting known from composting, which is nothing more than an exothermic oxidation of the dissolved straw components and in particular the atmospheric carbon with atmospheric oxygen. Both together are also referred to as soil respiration, which ultimately produces (atmospheric) carbon doxide (CO₂) and water (H₂O). This carbon dioxide can be described as atmospheric because both its carbon content and its oxygen content are of atmospheric origin. The atmospheric CO₂ escapes from the soil into the atmosphere, creating a carbon cycle.

A (smaller) part of the atmospheric carbon stemming from the straw incorporated into the arable soil does not oxidize to form CO₂ for a short time during soil respiration, but remains in the soil as a component of the so-called active nutrient humus for a period of time (a maximum of several decades) with digressively decreasing proportions. In contrast, passive permanent humus remains in the soil for centuries and millennia.

Humus is a complex mixture of living and dead organic matter which is contained mainly in the topsoil of fields. This organic soil substance (OSS) is the basis of life for heterotrophic soil organisms. Stray materials of vegetable origin, such as chopped straw, and straw materials of animal origin, such as farm manure (liquid manure, manure), are converted by fungi, representatives of macrofauna (e.g. earthworms, woodlice, centipedes) or representatives of mesofauna (e.g. enchytrias, collembolan) in a permanent process of degradation, conversion and build-up. Already comminuted plant and animal remains as well as excrements of the soil animals are further degraded by secondary decomposers (bacteria, fungi).

According to the current opinion, humus consists of relatively short-chain substances of various types (polysaccharides, polypeptides, aliphatic groups (fats), polyaromatic lignin fragments) which form so-called aggregates with cations, sand and clay particles.

Long-term permanent experiments have shown that the humus content has positive effects on the chemical, physical and biological properties of a sol. When comparing two extreme variants, which indicate the upper and lower limits of the change of the respective characteristic, the content of organic soil substance (OSS) was increased from a clear deficiency to the upper limit of normal arable possibilities. There were significant positive effects on storage density, pore volume and aggregate stability and consequently a significantly improved water infiltration, water storage capacity and usable field capacity. In addition, soil life was intensified, and the earthworm density and microbial biomass increased significantly. This also increased the nutrient content (N, P, S) of the soil, the proportion of trace elements and the cation exchange capacity. The supply of convertible organic matter led to an increased release of nutrients, from which especially light soils benefited, whose yield capacity increased on the average by 10%-33% and in maximum by about 125%.

Experts therefore come to the conclusion that the supply level of soil with organic matter or the humus content of the soil can be regarded as superior characteristics for soil quality because a broad spectrum of important soil fertility properties depend directly or indirectly on them. Thus, humus content and soil fertility are more or less equated.

The humus reserves of the soil are simply divided into two fractions of different (bio-)chemical stability and lifetime, namely the active and unstable part of the nutrient humus and the passive and stable part of the permanent humus. The greater part of the humus reserves of the soil has been formed in a process of soil formation that has been ongoing since the last ice age. It is chemically very stable and is therefore also called permanent humus. Degradation products from the suppled organic matter form strong bonds with the clay and fine silt particles of the soil, so that further degradation of this OSS is prevented in the long term. Permanent humus is therefore characterized by residence times of hundreds to thousands of years. This largely stable humus fraction comprises about 20%-50% of the entire humus stock in light soils and up to over 80% in heavy soils.

The organic substances supplied in the form of harvest and root residues as well as farm manure liquid manure, manure) belong to the so-called active nutrient humus, i.e. an unstable, partly highly unstable humus fraction. This nutrient humus is subject to permanent biochemical transformation processes, which proceed more or less quickly.

After the incorporation into the soil, the biochemically very easily degradable components of the freshly added organic material are first used by the soil organisms (animals, microorganisms, fungi) as a source of food and energy on a short-term basis —usually within a few months—and then consumed into carbon dioxide. These materials include in particular those with close C/N relations, such as green manure. More difficult to degrade organic substances, such as harvest and root residues, with wide C/N ratios and high lignin contents, such as straw which has a C/N ratio of 70/1 to 100/1 (on the average 86/1), as well as residues and metabolites of soil organisms initially accumulate in the soil to some extent until they are finally degraded after a few decades. Ultimately, all added organic substances that are not permanently chemically stabilized are completely degraded within 25-30 years.

The decomposed organic substance is finally released into the soil as (seepage) water and into the atmosphere as carbon dioxide. The nutrients introduced with the plant and animal materials (above all the functional elements, i.e. potassium and sodium) are predominantly released already in the first year after the introduction into the soil because they are not built into the plant cell structures. The organically bound basic nutrients, i.e. nitrogen, phosphorus and sulfur and some trace elements, are largely released only in the medium term and are then available for plant growth. This release of the chemical building materials is called mineralization.

Depending on the respective environmental and site conditions, more or less rapid degradation, conversion and build-up processes take place in the humus portion of the soil. For example, the humus composition is determined by the type and quantity of harvest and root residues, by the quantity of dead soil animals and microorganisms and by organic fertilizers, which are also referred to as organic primary substances (OPS). How long these OPS remain in the soil depends on the degradation intensity or degradation resistance of the OSS.

The current humus content (=the supply level of organic matter to top soil) can be regarded as an open flow equilibrium between the supply and degradation of OSS. In order to maintain a certain humus level, the organic substance dissolved by mineralization must be replaced continuously or annually by the addition of newly formed harvest and root residues and/or new organic fertilizer. Only then Is the humus balance balanced.

On the one hand, the conversion of OSS thus depends on the living conditions (weather conditions, soil properties) of the organisms involved in degradation and, on the other hand, on the properties of the substrate to be decomposed (consistency, C/N ratio, degree and type of stabilization). The degree of OSS stabilization Is, in turn, dependent on the site-specific soil characteristics and the properties of OSS. Some form of OSS stabilization interacts with minerals from the clay fraction (clay minerals, iron oxides). In the process, so-called clay-humus associates are formed. These compounds are so stable that they largely protect the atmospheric carbon of the plant from oxidation by microorganisms and from other chemical reactions.

In addition, the humus also is used as a store and transformer of nutrients, in particular nitrogen, sulfur and phosphorus; a gradual release of these nutrients allows an improvement in nutrient utilization. Humus is also used as a buffer and filter, as it can immobilize toxic substances and partially detoxify them.

Humus consists essentially of organic carbon, organic oxygen, organic hydrogen, organic nitrogen, organic phosphorus and organic sulfur. These elements also occur in soil in inorganic compounds. An analytical separation between the organic and inorganic components is only possible for carbon. For this reason, the organic carbon content is used as a yardstick for the humus content of a soil.

Humus dry substance consists of 30%-70% (weighted average 58%) of organic and thus of atmospheric carbon. With this mass fraction, carbon is the most important component of soil organic matter (OSS).

The humus content and the associated carbon content of the uppermost soil layer (top soil) are 1%-4% humus in arable land (with an average C content of humus of 58%, i.e. 0.58%-2.32% humus-C_{org}), 2%-8% humus in forest areas (1.16%-4.64% humus-C_{org}) and 4%-15% in grassland (2.32%-8.7% humus-C_{org}). According to the Soil Mapping Guide KA-2005 of the Soil Working Group of the German Federal Institute for Geosciences and Natural Resources (FGR), the humus and C contents of the uppermost soil layer are divided into 7 levels.

Relative to time, the degradation of OSS and thus of carbon takes place digressively depending on the substrate: when peat and wood are applied, 75% of the first C dose tends to still be present in the soil after 1 year, 65% after 2 years and 60% after 3 years. Green manure is much more short-lived: after 1 year only 15% is still present, after 2 years only 10% and after 3 years only 5%. Of the native straw worked into the soil, only 35% remains after 1 year, 25% after 2 years and 15% after 3 years. After 25-30 years there is usually nothing left of an OSS dose in the soil other than the degradation end products, i.e. water (H₂O) and carbon dioxide (CO₂), and even these are usually seeped away or evaporated after this time.

The degradation rate of OSS is determined inter ala by the C/N ratio, which is almost ideal for green manure with a ratio of 20 for soil organisms, for straw with 70-100 (on the average 86) already much further precipitates and for peat/wood with up to 300 is extremely wide. After the supply of biomass into the soil, an aerobic physico-chemical rotting process begins in addition to the utilization by soil organisms. As in composting, this process reduces part of the biomass in an exothermic oxidation process. This means that a (highly unstable) part of the freshly supplied biomass is decomposed within a few weeks and months. Only the unrotten remainder of the supplied biomass enters the unstable pool of the nutrient humus for a longer period of time (1-30 years). Accordingly, there are specific humification factors for each substrate, the so-called humus equivalents HEQ, which are measured in kg of humus equivalents/ton of substrate, wherein there is HEQ for both the wet mass and the respective dry mass. With high applications on organic matter, the HEQ factors tend to be lower than with low feed quantities because the degradation rate is higher with high doses.

For straw with a wet mass content of 86%, humification values of 41-83 kg HEQ/t straw-WM are given (on the average 62 HEQ/t straw-WM) and for straw dry substance 48-97 kg HEQ/t straw dry substance (on the average 72 HEQ/t straw-DS), i.e. a relatively low humus effect occurs in the soil during the incorporation of straw. When applying the rather solid phase from the liquid fermentation residues of the fermentation of cattle and pig manure (DS content of this solid phase 25%-35%), HEQ values of 24-46 kg HEQ/t WM and 95-133 kg HEQ/t DS were determined. In order to determine the carbon content of humus, these HEQ values must be divided by the average factor of 1.724 or multiplied by the average percentage of 58%, wherein it should be noted that the range of the carbon content is 30%-70% depending on the location, which corresponds to HEQ factors of 3.3-1.4.

With its 41-83 kg HEQ/t straw-WM, straw delivers only about 12-58 kg C_{org}/t straw-WM (on the average 35 kg C_{org}/t straw-WM) into the soil, in consideration of the entire range, and the rather solid phase of the above mentioned fermentation residues only 7-32 kg C_{org}/t WM (on the average about 20 kg C_{org}/t WM). Due to the wide C/N ratio and the resulting resistance to degradation, straw is nevertheless relatively well suited as a humus-forming agent, as it takes a relatively long time compared to other organic substrates until the soil flora and soil fauna have completely decomposed it.

The carbon, which is mostly bound in carbohydrates and thus not chemically stabilized, is used by soil organisms to generate energy. Nitrogen and other nutrients and micro-nutrients use them to build up substances. The end products of energy utilization (carbon doxide and water) leave the field top soil by evaporation and seepage (see above), while the (organic) nitrogen becomes part of the microbial biomass and thus of the humus. With the degradation of carbon and the simultaneous storage of nitrogen in the microbial biomass, the C/N ratio of humus (or OSS) gradually narrows to the 6.6-30.0 ratios commonly found in agricultural soils.

As a rule, high soil humus contents are associated with high soil biological activity which has positive phytosanitary effects and tends to reduce the need for plant protection products. High humus contents usually result in increased aggregate stability, good soil aeration, improved water storage, increased rootability, reduced soil erosion, lower surface runoff and the reduction of harmful soil compaction.

Under constant environmental and vegetation conditions, a balance between delivery and degradation of organic matter is achieved on arable soils in the medium term. With a constant annual supply of organic materials (e.g. In the form of a constant supply of straw), the associated increase in the degradation rate results in a further accumulation of active nutrient humus that decreases from year to year. The cumulative overall effect shows a decreasing boundary effect. The increase in the degradation rate is due to the fact that humus degradation is a function of the total nutrient humus stock.

After about 20-30 years, the cumulated humus content does no longer increase ceteris paribus. The respective soil has reached a so-called flow equilibrium in which the supply from organic fertilization corresponds exactly to the amount of organic substance that is degraded annually by mineralization. The organic fertilizer from the first year of application was completely degraded at this time. This results in a new site-specific humus level. Accordingly, an increase in the supply level of organic materials leads to an increase in the humus supply and the humus content of the soil. Ceteris paribus, humus degradation then also increases until a new, higher humus flow equilibrium is reached in the soil, i.e. the humus supply quantity again corresponds exactly to the humus degradation quantity.

Up to now, however, if arable land is used continuously, cultivation measures have had only limited effects of a few tenths of a percentage point on the humus and thus on the carbon content of soils. In Europe, organic carbon accounts for 0.23%-9.45% of the mass of agricultural soils. The European average for very light sandy soils (clay content <5%) is about 0.87% C_{org} on the average, for light sandy soils (clay content 5-12%) about 1.01% C_{org} on the average, for medium heavy soils (clay content 12-25%) about 1.39-1.52% C_{org} on the average, for heavy soils (clay content 25-45%) about 1.56% C_{org} on the average and for very heavy soils (clay content >45%) about 2.01% C_{org} on the average. C_{org} and humus contents therefore increase with increasing clay content. However, specific site conditions such as high precipitation in particular can lead to sandy soils with high humus contents and clay soils with low humus contents.

Despite its relatively low HEQ and carbon values (see above), the straw produced during the grain harvest as an agricultural by-product is currently the most important organic fertilizer for the humus reproduction of arable land. Grain straw is therefore not regarded as residual material or even as waste from arable land use. Accordingly, it is recommended to avoid straw removal from areas with negative humus balances over several years.

In order to promote the conversion of straw worked into the soil into C-containing humus, fertilization with nitrogen (N) is often carried out during its incorporation into the arable soil. Because of the high C/N ratio of 70/1 to 100/1 (on the average 86/1) for grain straw, the microorganisms are initially unable to process the straw property because they lack the proportionate nitrogen required to build up the body's own proteins in this C/N ratio.

The optimum C/N ratios for soil microbes are about 6-10, which are produced approximately with the addition of nitrogen.

According to Cross Compliance (DirektZahlVerpflV 2004), straw humification with 100 kg C-containing humus per ton of straw wet mass is to be assumed, which corresponds to about 58 kg C_{org}/t straw wet mass with an average humus-carbon content of 58% (range: 30-70 kg C_{org}). According to the humus balance of the Association of German agricultural testing and research institutes (VDLUFA), straw is evaluated with a humus reproduction capacity of 80-110 kg HEQ per ton of straw wet mass, which, when the full range is used, corresponds to a carbon content of 24-77 kg C_{org}/t straw-WM and an average of about 50 kg C_{org}/t straw-WM. According to the specifications of the Bavarian State Institute of Agriculture, which is based on the so-called humus unit method, Bavarian straw farms should assume a humus reproduction capacity of 70 kg HEQ (41 kg C_{org}) per ton of straw wet mass.

In the following, an average humus reproduction capacity of 35 kg C_{org} per ton of straw wet mass is assumed for straw with 100% retention of the straw on the field, which equals a humus equivalence of about 60 kg HEQ with an average carbon content of 58%.

An energy-related utilization of straw, such as straw combustion in straw-fired combined heat and power plants, means that it is no longer available for soil humus reproduction.

With cereal-based crop rotations and an assumed humus reproduction capacity of 60 kg HEQ per ton of straw wet mass (35 kg C_{org}/t straw-WM), a sustainable humus reproduction capacity is only assumed in the federal state of Brandenburg if at least 50% of the straw remains in the field. In its detailed study “Basisinformation für eine nachholtige Nutzung von landwirtschaftlichen Reststoffen zur Bioenergiebereitstellung—DBFZ-Report Nr. 13” (Basic Information for the Sustainable Use of Agricultural Residues for the Provision of Bioenergy—GBRC report no. 13), the GBRC determined in 2012 using the dynamic humus unit method that in order to maintain the humus content of arable soils an average of 65% of the straw growth must remain in the field. This means that only 35% of the national straw growth is available for the material and energetic use. In the past, about half of this (18%) was used as bedding for livestock and horse husbandry, so that only about 17% of the straw growth was effectively available for energy use. If it is assumed that the straw used as bedding is completely returned to the field in the form of straw-containing manure (solid manure) (no energetic utilization of the solid manure in biogas plants), the proportion of the national straw growth available for energetic utilization increases to 35% or to ⅓. This means that about ⅔ of the German straw growth must remain in the field.

### 3 PRIOR ART

At present, there are essentially three technical directions that carny out CO₂ recuperation in order to produce emission-reduced energy carriers from coal, natural gas or crude oil: firstly, the post-combustion capture technology; secondly, the overstoichiometric combustion (oxyfuel technology); and thirdly, pre-combustion capture technology.

The post-combustion capture technology is mainly used in coal-fired power plants that burn fossil coal to generate electricity in order to capture and recuperate (fossil) CO₂ from their flue gas in order to reduce the CO₂ emission rate of the coal-fired power plant and the GHG emission value of the generated electric current. US 2007/0178035A1 (White/Allam), DE102008062497A1 (Linde-KCA-Dresden) and DE102009043499A1 (Uhde) are known to have techniques for extracting fossil CO₂s from flue gases from combustion plants, especially from large power plants operated with fossil carbon or fossil natural gas. The extracted fossil CO₂ shall be sent to geological final disposal site (so-called CCS process, CCS standing for Carbon Capture and Storage). The further and additional pollution of the atmosphere of the earth with additional fossil CO₂ shall thus be reduced and in the ideal case completely avoided.

When the post-combustion capture technology is applied, existing deposal sites of fossil energy carriers are still exploited with increasing extraction or production rates. The conversion of these fossil energy carriers into marketable energy carriers is not at all sustainable, only the emission of fossil CO₂s—i.e. the emission rate—is reduced. In addition, for technical and economic reasons, it is not possible to completely capture and finally store the fossil CO₂s generated by the combustion of fossil energy carriers. This means that even when using the best CCS processes and CCS plants, there is always a certain CO₂ slip, which is between 2% and 85% of the CO₂ volume and thus further increases the stock of greenhouse gases in the atmosphere of the earth.

With the CCS technologies designed and planned so far, neither an absolutely GHG-emission-free nor a sustainable coal or natural gas stream can be produced, nor can GHG-neutral fuels such as hydrogen, methanol, ethanol or synthetic methane, butane, octane, propane or DME be produced because the CCS processes still use fossil carbon. Neither these processes know atmospheric carbon nor its chemical-physical stabilization. Nor do they know of any carbonization of atmospheric carbon-containing residues from a biomass conversion to a biochar/vegetable coal/biocoke consisting of atmospheric carbon. They certainly do not disclose the incorporation of chemically and physically stabilized carbon in agricultural soil to maintain or improve the humus content of this soil.

They do not at all describe the combination of their system with a fuel, heating medium or combustion material production and utilization system that combines GHG-positive GHG emission quantities of a fuel, heating medium or combustion material determined according to LCA with GHG-negative emission quantities of another compatible fuel, heating medium or combustion material also determined according to the LCA method to a balanced emission zero quantity of a corresponding fuel, heating medium or combustion material mixture.

The oxyfuel process, for which WO2004/094901A1 (Abrams & Culvey) can be cited as an example, is also previously known. WO2004/094901A1 teaches a two-stage combustion of solid carbon-containing combustion materials with pure oxygen and nitrogen-free accompanying gases, such as argon and carbon dioxide, wherein a fuel gas is initially produced under nitrogen-free conditions. This fuel gas is burned (oxidized) with overstoichiometric addition of pure oxygen and argon to generate heat and convert it into steam in a boiler. The flue gas from this overstoichiometric combustion, which contains a high percentage of CO₂ and is largely free of nitrogen oxides, is freed from fly ash and dust using the cyclone technology. The resulting exhaust gas is freed from gaseous salts by means of an acid gas scrubber” and fed to a cryogenic CO₂ separation plant. The resulting pure CO₂ is returned to the first (or second) combustion stage to control or regulate the process to a lesser extent, while the generated CO₂ is used as an industrial product to a greater extent. WO2004/094901A1 mentions biomass as a combustion material, but means all hydrocarbon-containing solid combustion materials, including crude oil-based used tires and crude oil-based plastics.

WO2004/094901A1 correctly states that any combustion of hydrocarbons produces the greenhouse gas, i.e. carbon dioxide, but that the only use of the captured CO₂ is as an industrial product. This means that WO2004/094901A1 does not disclose any process or system with which atmospheric carbon could be chemically-physically stabilized. Nor does WO2004/094901A1 describe the chemical-physical stabilization of atmospheric carbon in order to avoid a reaction with atmospheric oxygen to form (atmospheric) CO₂, either as a method or a device. WO2004/094901A1 also does not describe any carbonization of atmospheric carbon still contained in conversion residues into biochar/vegetable coal/biocoke. WO2004/094901A1 certainly does not describe the incorporation of atmospheric carbon, which is chemically and physically stabilized, into agricultural soil in order to maintain or improve the humus content of this soil. Nor does WO2004/094901A1 teach a method that permanently removes atmospheric carbon as such from the atmosphere of the earth, neither a method for geological sequestration of atmospheric carbon as such nor the material substitution of fossil CO₂s by atmospheric CO₂, nor the production of GHG-emission-reduced synthetic methane from GHG-emission-reduced hydrogen and atmospheric CO₂, nor its use as a substitute for one or more fossil fuels. The subject matter of WO2004/094901A1 is also not suitable for this purpose, because WO2004/094901A1 neither distinguishes between fossil and atmospheric carbon nor between fossil and atmospheric CO₂—which is an essential basis for the invention and for the evaluation of the invention—nor are the apparatuses of WO2004/094901A1 suitable for ensuring that the total amount of greenhouse gases in the atmosphere of the earth does not increase further despite the production of fuels, heating mediums or combustion materials. Nor does WO2004/094901A1 teach how to optimize the overall process of producing, distributing and using fuels, heating mediums or combustion materials in terms of greenhouse gas emissions. WO 2004/094901A1 certainly does not describe the combination of its system with a fuel, heating medium or combustion material production and utilization system which combines the GHG-positive GHG emission quantities of a fuel, heating medium or combustion material determined according to LCA with the GHG-negative emission quantities of another compatible fuel, heating medium or combustion material mixture also determined according to the LCA method to form a balanced GHG total quantity of a corresponding fuel, heating medium or combustion material mixture.

So-called ore-combustion capture processes and corresponding PCC systems are also previously known, which in so-called IGCC (Integrated Gasification Combined Cycle) power plants sub-stoichiometrically convert fossil energy carriers, such as coal, but also regenerative energy carriers, such as biomass or waste, into a (combustion) gas consisting essentially of hydrogen and carbon monoxide in partial oxidation with water using the gasification technology. Using suitable catalysts, the carbon monoxide (CO) is allowed to react under high pressure with water vapor to form CO₂ and hydrogen (H₂). At pressures of up to 70 bar, the reaction product, i.e. CO₂, can be relatively easily absorbed from the gas mixture using one of the known processes (pressure swing absorption, amine wash, cryogenic technology, etc.). The (combustion) gas freed from CO₂ is converted into electricity with high efficiency in gas turbines, but it can also be used to produce pure hydrogen, methanol, ethanol or synthetic methane, octane, propane, butane or DME. Since the CO₂ is removed from the (combustion) gas before it is used, this technology is also known as “pre-combustion capture” technology. However, the by-product of the PCC process is only CO₂ and not coal or coke. IGCC power plants have a GHG emission value that is up to 15% lower, which makes them appear relatively clean compared to conventional coal-fired power plants, but their GHG emission reduction performance is only 15% and not 100%. IGCC does not achieve GHG-negative emission values at all.

CN102784544 (A) (XU et al.) is an example of such a pre-combustion capture technology. This published patent application, which comes under the field of “clean coal power generation”, describes a system for the PCC recuperation of CO₂ prior to the use of the (combustion) gas generated. The invention relates to an IGCC-based pre-combustion CO₂ capture system comprising a sulfur-resistant conversion apparatus, an MDEA (methyl d ethanol amine) desulfurization and decarburization device, and a sulfur and carbon separator. The sulfur-resistant conversion device is converted into a mixed gas consisting mainly of CO₂ and H₂, as described above, to convert the CO contained in the synthesis gas, said conversion being carried out in a conversion furnace. The MDEA desulfurization and decarburization device comprises an absorption tower and a desorption tower. The absorption tower is used to receive the mixed gas in the sulfur-resistant conversion device and to absorb CO₂ and H₂S gases and the desorption tower is used to receive CO₂ and H₂S and to desorb CO₂ and H₂S. The sulfur and carbon separator comprises a desulfurization cleaner to receive the CO₂ and H₂S gases in the desorption tower. And the H₂S gas is absorbed by an H₂S absorber, leaving the CO₂ gas behind. A further effect of the invention is that the concentration of CO₂ in the mixed gas can be increased to 35%-45%, which makes the absorption of CO₂ technically less complex and thus more cost-effective.

CN102784544(A) does not differentiate between fossil and atmospheric carbon nor between fossil and atmospheric CO₂—which is an essential basis for the invention and for the evaluation of the invention. The PCC technology of CN102784544(A) does not produce any chemically and physically stabilized carbon, especially no chemically and physically stabilized atmospheric carbon. CN102784544(A) also does not teach a carbonization of atmospheric carbon still contained in conversion residues. The teaching of CN 102784544(A) certainly does not cover the incorporation of atmospheric carbon, which is chemically and physically stabilized, into agricultural soil in order to maintain or improve the humus content of this soil. The PCC technology does not include the combination of a PCC system with a fuel, heating medium or combustion material production and utilization system that combines the GHG-positive GHG emission quantities of a fuel, heating medium or combustion material determined according to LCA with GHG-negative emission quantities of another compatible fuel, heating medium or combustion material also determined according to the LCA method to form a balanced zero quantity of a corresponding fuel, heating medium or combustion material mixture.

These three technologies for CO₂ recuperation (post-combustion capture technology, oxyfuel process, pre-combustion capture process) have in common that, firstly, no distinction is made between fossil and atmospheric carbon or between fossil and atmospheric CO₂ and that, secondly, the aspect of chemical-physical stabilization of atmospheric carbon is not addressed. Moreover, in the end only fossil CO₂ gas is available in addition to the energy carrier produced and no (more or less pure) carbon. This lacks the basic prerequisites for the production of a GHG-negative fuel, heating medium or combustion material, which in turn is a prerequisite for the production of a mixed fuel consisting of GHG-negative and GHG-positive components or mixed heating medium or combustion material mixture with balanced GHG balance or with a GHG emission value of zero.

DE19747324C2 (Wolf) describes a device for producing combustion, synthesis and reduction gases from fossil and renewable combustion materials, biomass, waste and sewage sludge by pyrolysis (CHOREN process). The carbonization gas, which has a temperature of up to 500° C., and the residual coke resulting from the carbonization are converted into combustion, synthesis or reduction gases in a reaction chamber at 500° C. to 1,200° C., leaving only mineral slag which no longer contains carbon. DE19747324C2 knows neither the difference between fossil and atmospheric carbon nor the difference between fossil and atmospheric CO₂—which is an essential basis both for the invention itself and for the evaluation of the invention. Accordingly, DE19747324C2 cannot indicate atmospheric carbon, let alone chemically and physically stabilized atmospheric carbon. This is simply not possible because all the carbon contained in the input materials supplied is converted into gas in DE19747324C2 and the (hazardous) slag, which is the only conversion residue, contains virtually no carbon. It is therefore not possible to avoid a reaction between (atmospheric) carbon and atmospheric oxygen. Accordingly, DE19747324C2 cannot remove CO₂ from the atmosphere of the earth and therefore cannot decarbonize the atmosphere of the earth. Even if only biomass such as straw were used, the atmospheric carbon contained in the straw would return to the atmosphere of the earth. Without sequestration of atmospheric carbon, the synthesis gas produced thus at best achieves GHG neutrality and no GHG negativity, and this only if it is ruled out that further greenhouse gas emissions are produced during the production, distribution and use of the combustion gas or the fuels and heating mediums produced from synthesis gas. Under no circumstances can DE19747324C2 provide biochars, vegetable coals or biocoke that would be suitable to maintain or even improve the humus content of agricultural or other soils.

Also previously known is the Bioliq® process of the Karlsruhe Institute of Technology (http://www.bioliq.de/55.php), which is used to produce synthetic fuels and chemical basic products from dry biomass. The Bioliq® process comprises five process steps: rapid pyrolysis, energy densification, high-pressure entrained-flow gasification, gas purification and fuel synthesis. The end products are designer fuels, such as syn-diesel, syn-gasoline, syn-kerosene, syn-DME and syn-methanol produced from synthesis gas by the Fischer-Tropsch, methanol or dimethyl ether synthesis. In addition to these fuels, the synthesis gas can also be used to produce many basic substances for the chemical industry. The developers of the Bioliq® process state the following advantages of synthetic fuels produced from biomass over other biofuels and over synthesis products produced from coal in the CtL process or from natural gas in the GtL process: Conservation of fossil raw materials; partial independence from energy imports; broad range of raw materials; no competition regarding use or land for food production; contribution to strengthening regional agriculture; existing infrastructure can be used without change; no change in vehicle technology necessary; provision of a wide range of fuel types (syn-diesel, syn-kerosene, syn-gasoline) possible; tailoring (“designer fuels”) to different engine types possible; no change in driving habits (range) required; reduction of anthropogenic CO₂ emissions.

In rapid pyrolysis (www.bioliq.de/64.php), the chopped biomass is converted into hot carbonization gas (pyrolysis vapors) and fine coke within seconds at 500° C. in a twin-screw reactor. For rapid heating of the biomass, a heat carrier circuit is used in which a 5-10-fold excess of sand is mixed with the biomass in the reactor. The pyrolysis vapors are cooled to ambient temperature by quench cooling and thus liquefied to a heavy oil-like aqueous condensate (slurry). What remains Is a carbonization tar and a combustible pyrolysis gas, which essentially consists of carbon dioxide, carbon monoxide and hydrocarbons and is burnt together with part of the pyrolysis residue contained in the sand—a fine coke. The resulting flue gas heats the sand circulating in the cycle. With optimum process control, only 10% of the energy contained in the biomass is allegedly required for rapid pyrolysis. The product of rapid pyrolysis is the energy-compacted flowable heavy oil-like condensate. In energy densification (www.bioliq.de/66.php), powdery pyrolysis coke and the pyrolysis condensate are mixed to form a suspension known as bioliq-syncrude. In high-pressure entrained-flow gasification (www.bioliq.de/67.php), the bioliq-syncrude is atomized in an entrained-flow gasifier with the addition of hot oxygen and converted at over 1200° C. into a tar-free, low-methane raw synthesis gas. The type of gasifier used for this purpose is particularly suitable for ash-rich biomass, such as straw. The reaction takes place under pressures, which are determined by the subsequent synthesis. Fischer-Tropsch syntheses require process pressures up to 30 bar, methanol or dimethyl ether syntheses (DME) up to 80 bar. The Bioliq® pilot gasifier installed in the demonstration project is designed for 5 megawatts (1 t/h) and two pressure stages of 40 and 80 bar and is based on the Lurgi Multi-Purpose-Gasification (MPG) concept. As by-products, heat and electricity are generated which cover a large part of the process energy and thus contribute to the required high CO₂ emission reduction performance. High-pressure high-temperature processes developed by KIT are used for gas cleaning (www.bioliq.de/69.php). These processes allow us to expect energy savings through optimum temperature control or heat displacement in the event of subsequent commercialization. In the first expansion stage of the Bioliq pilot plant, a particle separation (ash, coke, soot) with ceramic filter cartridges will first be carried out at 800° C. Then, acid gases (HCl, H₂S), alkalis and heavy metals are separated at about 500° C. in a fixed bed absorber with trona as sorbent (NaHCO₃, Na₂CO₃×2H₂O). A downstream catalytic converter is used to decompose organic and nitrogen-containing substances (HCN, NH₃). In the first expansion stage, CO₂ is separated by conventional solvent wash; hot gas wash is also planned for a later development stage of the plant. The separated CO₂ is reused internally in the fuel process for the fuel synthesis. The fuel synthesis (www.bioliq.de/73.php) takes place in two stages via dimethyl ether (DME) as an intermediate product, for the synthesis of which a hydrogen to carbon monoxide ratio of about 1:1, as usually occurs in biomass gasification, is advantageous. The DME synthesis takes place at about 250° C. and a pressure of about 55 bar. In the pilot plant, the DME is directly converted into a high-octane motor gasoline. Here, zeolite-catalyzed dehydration, oligomerization and isomerization take place at temperatures of about 350° C. and a pressure of about 25 bar. Based on known processes (MtG Methanol-to-Gasoline), this results in a fuel with high selectivity and gasoline quality. Unreacted synthesis gas is returned to the reactor via a gas recirculation system.

In the KIT Bioliq process, decentralized biogenic residues from agriculture and forestry, such as straw and thinning wood, shall be used. According to (www.bioliq.de/212.php), all types of dry biomass with less than 15% water, including those with high ash contents such as straw, and fast-growing biomass, such as wood from short-rotation plantations, are suitable as input materials. The highly complex process of “entrained flow gasification-gas purification-fuel synthesis” is, however, only economical when large industrial units are used (economies of scale). With the required biomass volume flow, the catchment area of these large industrial units similar to refineries is very large. In order to save allegedly too expensive transport routes, the entire process was therefore divided into a smaller decentralized pre-treatment for energy densification of the biomass and large plants for further processing of the product (bioliq-syncrude) produced in a decentralized way. In order to produce 1 kg fuel (with a calorific value of 12.0 kWh_{Hi}), the use of 8-10 kg biomass is required (32.7-40.9 kWh_{Hi}). This means that the conversion efficiency is merely 29%-37%. The biocoke produced in the process is used either for heating the fast pyrolysis or as part of the bio-syncrudes. The (atmospheric) CO₂ produced in the process is used in the fuel synthesis. Apart from tar, which is harmful to health and can no longer even be used in road construction in Germany, the Bioliq® process does not produce any carbon-containing conversion residues, so that the Bioliq® process cannot be used to chemically and physically stabilize atmospheric carbon. This means that the process cannot provide biochar/vegetable coal or biocoke, nor can it provide atmospheric CO₂. Consequently, the 3 options of sequestration of atmospheric CO₂s, a substitution of fossil CO₂s by atmospheric CO₂ and the production of synthetic fuels, such as syn-methane, from atmospheric CO₂ are also eliminated. Without biochar/vegetable coal/biocoke, no stabilized atmospheric carbon can be sequestered. This means that fuels produced according to the Bioliq® process can at best achieve GHG neutrality, but not GHG negativity. The Bioliq® process cannot contribute to ensuring or improving the humus content of (agricultural or forestry) soils.

DE 10 2005 045 166 B4/EP 1943 463 B1 (Sehn & Gerber) previously discloses a procedure and devices for producing thermal energy, with which biomass, in particular grain or stalk-like energy carriers, are continuously fed to a pyrolysis reactor and in which the pyrolysis gas obtained in the pyrolysis reactor is fed to a FLOX burner for flameless oxidation and in which the waste gas from the FLOX burner preheats the combustion air of the FLOX burner, the waste gas from the FLOX burner being applied to the outside of the pyrolysis reactor. The biomass is converted into pyrolysis gas at about 500° C., which is then fed into the FLOX burner. The pyrolysis process converts about 70% of the energy content of the biomass used or about 85% of the dry fuel mass used into gas. The biocoke produced in the pyrolysis reactor as a by-product of the process is used for energetic utilization, wherein the biocoke is preferably used in a coal liquefaction process where it replaces fossil coal. DE102005045166B4/EP1943463B1 does not know the difference between fossil and atmospheric CO₂ and does not describe atmospheric carbon. The DE102005045166B4/EP1943463B1 also does not use any conversion residues from an upstream first conversion process. Furthermore, it does not reveal any chemical-physical stabilization of atmospheric carbon still contained in conversion residues in order to avoid a reaction with atmospheric oxygen to (atmospheric) CO₂, neither as a process nor as a device. DE102005045166B4/EP1943463B1 certainly does not disclose the incorporation of atmospheric carbon, which originates from the remains of an upstream first biomass conversion and which is chemically and physically stabilized, into agricultural soil in order to maintain or improve the humus content of this soil. DE102005045166B4/EP1943463B1 does not describe at all the combination of such a method or system with a fuel, heating medium or combustion material production and utilization system, which combines the GHG-positive GHG emission quantities of a fuel, heating medium or combustion material determined according to LCA with GHG-negative emission quantities of another compatible fuel, heating medium or combustion material also determined according to the LCA method to form a balanced zero quantity of a corresponding fuel, heating medium or combustion material mixture. The dry substance loss occurring in the method, which is 85%, is very high, i.e. only 15% biocoke and ash are left behind.

Patent specification EP1767658A1 (Griffin et al.) is also previously known. This document discloses a method for producing bio-ethanol from lignocellulose-containing input materials, such as straw (IOGEN process). This published patent application does not specify the whereabouts of the conversion residues. EP767658A1 does not know the difference between fossil and atmospheric CO₂ and does not describe atmospheric carbon. Furthermore, no chemical-physical stabilization of atmospheric carbon still contained in fermentation residues is described as a method or device to avoid a reaction with atmospheric oxygen to form (atmospheric) CO₂. EP1767658A1 certainly does not reveal an incorporation of atmospheric carbon which originates from the remains of an upstream first biomass conversion and which is chemically and physically stabilized, into agricultural soil in order to maintain or improve the humus content of this soil. EP1767658A1 does not even describe the combination of such a method or system with a fuel, heating medium or combustion material production and utilization system that combines the GHG-positive GHG emission quantities of a fuel, heating medium or combustion material determined according to LCA with the GHG-negative emission quantities of another compatible fuel, heating medium or combustion material also determined according to the LCA method to form a balanced GHG zero quantity of a corresponding fuel, heating medium or combustion material mixture.

Another method for producing bio-ethanol for the production of bio-ethanol from lignocellulose-containing biomass by alcoholic fermentation is described in US2002/0192774A1 (Ahring et al.). The method comprises 8 steps, namely 1.) conversion of the biomass into an aqueous suspension; 2.) heating of the aqueous suspension and/or transfer of the aqueous suspension from step 1 to an oxygen-enriched atmosphere to achieve at least partial separation of the biomass into cellulose, hemicellulose and lignin; 3.) at least partial hydrolysis of the cellulose and hemicellulose split off in step 2 with the aim of producing a sugar-containing suspension which is fermentable by microorganisms and is suitable as a starting material for the ethanol production; 4.) alcoholic fermentation of the sugar-containing suspension fermentable by microorganisms from step 3 into ethanol; 5.) separation of the ethanol from the fermentation mass resulting in a stillage containing substances that would inhibit alcoholic fermentation if returned to the process; 6.) treatment of the stillage with the aim of recycling the inhibitor concentration to such an extent that recycling the treated stillage into the process does not impair the alcoholic fermentation; 7.) recycling of the treated stillage or part thereof to one of steps 1 to 5; 8.) repeating steps 1 to 7. About 80% of the organic dry substance of the stillage can be degraded by an anaerobic bacterial digestion or by aerobic decomposition (oxidation), minimizing the amount of waste produced during the process. The (atmospheric) CO₂ produced during the process is released into the atmosphere of the earth. Consequently, the 3 options of a sequestration of atmospheric CO₂s, a substitution of fossil CO₂s by atmospheric CO₂ and a production of synthetic fuels such as syn-methane from atmospheric CO₂ are also eliminated. Since there is no carbon-containing conversion residue either, the US2002/0192774A1 method cannot be used to chemically and physically stabilize atmospheric carbon. This means that the process cannot provide biochar/vegetable coal or biocoke. Without biochar/vegetable coal/biocoke, no stabilized atmospheric carbon can be sequestered. Thus the ligno-ethanol produced according to the method can at best achieve GHG neutrality, but not GHG negativity. The process cannot even contribute to ensuring or improving the humus content of soils (used for agriculture or forestry) because there is no material from which carbon-containing humus, nutrient humus or permanent humus could be produced.

Furthermore, the Sunliquid® method of CLARIANT AG (formerly Süd-Chemie GmbH) Is known from (www.clariant.com/de/Innovation/Innovation-Spotight-Videos/sunliquid) and from (http://www.pflanzenforschung.de/de/joumal/joumalbeitrage/stroh-ist-nicht-gleich-stroh-interview-mit-dr-markus-ra-10584), which Is used to produce lingo-ethanol from plant residues (cereal straw, corn straw, bagasse) by means of enzymatic alcohol fermentation. The plant residues are first comminuted and thermally pre-treated without the use of chemicals. Enzymes the production of which Is integrated into the process and which are “tailor-made” for the respective input material then break down the hemicellulose and cellulose contained in the input material by means of enzymatic hydrolysis. Products are C5 and C6 sugars. What remains is lignin, which is unusable for ethanol production. This lignin is burned to generate the heat required in the process. The C5 and C6 sugars are converted by fermentative microorganisms into ethanol, which is available after this method step in such a way that it is dissolved in an aqueous suspension. The ethanol is concentrated and purified. The greenhouse gas reduction performance of the bio-ethanol produced shall reach 95%. CLARIANT intends to produce about 1,000 t of ligno-ethanol from about 4,500 t of straw. A calorific value input of 4,500 t×4,085 kWh_{Hi}/t=18,383 MWh_{Hi} is thus faced with a calorific value output of 1,000 t×7,467 kWh_{Hi}/t=7,467 MWh_{Hi}. The conversion efficiency is therefore only 7,467 MWh_{Hi}/18,383 MWh_{Hi}=40.6%, which is only slightly more than with the Bioliq® process of KIT (see above).

Since the straw mass is almost completely converted into sugar and the residual lignin is also used for the process, no atmospheric carbon is available for a chemical-physical stabilization. This means that the process cannot provide either biochar/vegetable coal or biocoke. Without biochar/vegetable coal/biocoke, no stabilized atmospheric carbon can be sequestered. Thus the ligno-ethanol produced by the method can at best achieve GHG neutrality, but not GHG negativity. The method cannot even contribute to ensuring or improving the humus content of soils (used for agriculture or forestry) because there is no material from which carbon-containing humus, nutrient humus or permanent humus could be produced.

Published patent application DE4332789A1 (Eliasson & Kiler) is also previously known. This document describes a method for storing hydrogen energy. A mixture of hydrogen and carbon dioxide is converted into methane and/or methanol in a reactor. Preferably, fossil carbon dioxide from the flue gas of fossil fuel-fired power generation plants is used. If required, the methane or methanol produced can be used as an energy carrier for vehicles, power plants and heating systems. As the inventors themselves state, the method is only suitable for reducing the GHG emission rate; due to the production of fossil CO₂s the amount of greenhouse gases in the atmosphere of the earth is still increased. This invention cannot reduce the GHG content of the atmosphere of the earth and therefore cannot cause GHG negativity, which is the prerequisite for compensating (positive) GHG emissions. Nor does DE4332789A1 describe any chemical-physical stabilization of atmospheric carbon to avoid a reaction with atmospheric oxygen to form (atmospheric) CO₂, either as a method or a device. DE4332789A1 certainly does not describe the incorporation of atmospheric carbon, which is chemically and physically stabilized, into agricultural soil in order to maintain or improve the humus content of this soil. DE4332789A1 does not even describe the combination of its system with a fuel, heating medium or combustion material production and utilization system that combines the GHG-positive GHG emission quantities of a fuel, heating medium or combustion material determined according to LCA with the GHG-negative emission quantities of another compatible fuel, heating medium or combustion material also determined according to the LCA method to form a balanced zero quantity of a corresponding fuel, heating medium or combustion material mixture.

Previously known DE102004030717A1 (Mayer) discloses a similar method and device by which geothermal and regenerative energy is converted into electrical energy and fed into a power grid, wherein a surplus of electrically generated energy is converted into a hydrocarbon and an alcohol using carbon dioxide and is stored as chemical energy in a container. The energy stored in the container is converted back into electrical energy for demand-based control in a power conversion process, while the surplus of chemically stored energy feeds a natural gas pipeline with synthetically produced methane and the surplus from converted electrical energy is used to generate hydrogen for a filling device. DE102004030717A1 does not distinguish between atmospheric and fossil CO₂. Thus this invention cannot reduce the GHG content of the atmosphere of the earth and thus also cannot cause GHG negativity, which is the prerequisite for the compensation of (positive) GHG emissions. DE102004030717A1 also does not describe any chemical-physical stabilization of atmospheric carbon to avoid a reaction with atmospheric oxygen to form (atmospheric) CO₂, neither as a method nor as a device. DE102004030717A1 certainly does not describe the incorporation of atmospheric carbon, which is chemically and physically stabilized, into agricultural soil in order to maintain or improve the humus content of this soil. DE102004030717A1 does not at all describe the combination of its system with a fuel, heating medium or combustion material production and utilization system that combines the GHG-positive GHG emission quantities of a (fossil) fuel, heating medium or combustion material determined according to LCA with the GHG-negative emission quantities of another compatible fuel, heating medium or combustion material also determined according to the LCA method to form a balanced zero quantity of a corresponding fuel, heating medium or combustion material mixture.

DE102009018126A1 (Stürmer et al.) of the Center for Solar Energy and Hydrogen Research (CSH) is also previously known. This document teaches an energy supply system in which a hydrogen production facility (electrolyzer) uses electricity from renewable sources (so-called renewable or RES electricity) for electrolysis and thus chemically binds it in the hydrogen energy carrier. This regenerative hydrogen is fed into a methanation reactor, into which a gas containing CO₂ is fed, wherein this CO₂ can be both fossil and atmospheric CO₂. The hydrogen gas and the CO₂ are synthesized to form methane in the methanation reactor according to the previously known Sabatier process. Published patent application DE102009018126A1 refers primarily to the renewable nature of the energies and energy carriers produced and not to the GHG emission reduction performance thereof. These two properties may well fall apart. In addition, the DE102009018126A1 method and system can at best be used to produce the GHG-neutral energy carrier “hydrogen gas” and only if absolutely GHG-neutral electricity and absolutely pure atmospheric CO₂ are used. Since DE102009018126A1 does not disclose a sequestration of atmospheric CO₂s nor a substitution of fossil CO₂s by atmospheric CO₂, this invention cannot reduce the GHG content of the atmosphere of the earth and thus also cannot cause GHG negativity, which is the prerequisite for the compensation of positive GHG emissions associated with the production, distribution and use of fossil fuels. DE102009018126A1 also does not describe any chemical-physical stabilization of atmospheric carbon to avoid a reaction with atmospheric oxygen to form (atmospheric) CO₂, neither as a process nor as a device. DE102009018126A1 certainly does not describe the incorporation of atmospheric carbon, which was chemically and physically stabilized, into agricultural soil in order to maintain or improve the humus content of this soil. DE102009018126A1 does not describe at all the combination of its system with a fuel, heating medium or combustion material production and utilization system which combines the GHG-positive GHG emission quantities of a fuel, heating medium or combustion material determined according to LCA with the GHG-negative emission quantities of another compatible fuel, heating medium or combustion material also determined according to the LCA method to form a balanced zero quantity of a corresponding fuel, heating medium or combustion material mixture.

US2010/0272619A1 (Frydman & Liu) is also previously known. This document describes a complex overall system consisting essentially of a gasification system, a so-called Water-Gas-Shift (WGS) reactor, a gas purification unit, a CO₂ dewatering and compression unit and a methanation unit. The output of the overall system is methane gas, which replaces natural gas and is therefore referred to by US2010/0272619A1 as Substitute Natural Gas (SNG). All carbon-containing substances, such as coal, oil coke, agricultural waste, wood-Ike materials, tar, asphalt and coke gas, are used as input materials. US2010/0272619A1 does not distinguish between atmospheric and fossil carbon. The carbon-containing fuels are converted in the gasification system under high pressure (20 bar to 85 bar), high temperatures (700° C. to 1,600° C.) and with the addition of pure oxygen by steam reforming into a so-called crude syngas, which essentially consists of carbon monoxide and hydrogen. Alternatively, carbonization of the carbon-containing fuels takes place in the gasification system at moderate reaction temperatures (150° C. to 700° C.), which are converted into carbon-containing coke ash (char) and a residual gas consisting of carbon monoxide, hydrogen and nitrogen. The coke ash produced by carbonization can react with carbon dioxide and water vapor to form carbon monoxide and hydrogen. The product is a crude syngas consisting of about 85% of carbon monoxide and hydrogen as well as CH₄, HCl, HF, NH₃, HCN, COS and H₂S. The crude syngas is fed into the WGS reactor where carbon monoxide reacts with water to form carbon doxide and hydrogen. The crude syngas enriched with hydrogen is fed to the gas cleaning unit. This unit removes unwanted gas components, such as HCL HF, COS, HCN and H₂S, from the crude syngas. The product is a purified syngas with a content of about 55% hydrogen, about 40% carbon dioxide and about 3% carbon monoxide. The gas cleaning unit can include a CO₂ separation system which also removes the carbon doxide contained in the crude syngas in too small a proportion (<2%) from the crude syngas. The carbon dioxide separated from the crude syngas is transferred to the CO₂ dewatering and compression unit, which dewaters and compresses it. The dewatered and compressed CO₂ is stored or used. It can be supplied through a pipeline to a sequestration plant, e.g. to a so-called EOR plant, which uses the CO₂ to better exploit oil deposits (enhanced oil recovery) or to a plant, which stores it in geological layers with saline groundwater. The purified syngas is fed to a methanation unit, which reforms the hydrogen and carbon monoxide in an exothermic reaction to form methane (CH₄) and water (H₂O). The heat contained in the methane and water is transferred via heat exchangers to water which is converted into high-temperature steam. Electricity is generated from the high-pressure steam. The synthetic methane (syn-methane) produced in the methanation unit is fed to the CO₂ dewatering and compression unit, which dewaters and compresses the syn-methane and feeds it into a (special) SNG pipeline for further use. This pipeline can be used to transport the syn-methane to a gas reservoir or to a methane-processing industrial plant.

Since US2010/0272619A1 uses coal and crude oil-based substances such as oil coke, tar, asphalt and coke gas as input materials, fossil carbon enters the atmosphere of the earth, and therefore a reduction of the CO₂ content of the atmosphere of the earth is simply not possible. US2010/0272619A1 states the purpose of sequestration for the CO₂ produced by the system but does not distinguish between fossil and atmospheric carbon or between atmospheric and fossil CO₂—which is an essential basis for the invention and for the evaluation of the invention. The US2010/0272619A1 system can therefore at best reduce the emission of additional fossil carbon or CO₂s into the atmosphere (i.e. the positive GHG emission rate) but not the CO₂ content in the atmosphere of the earth. The latter would require a negative emission rate. For the compensation of positive (fossil) CO₂ emissions into the atmosphere of the earth, a negative CO₂ flow or an absolute reduction in the carbon level in the atmosphere of the earth or the removal of CO₂ from the atmosphere of the earth are mandatory prerequisites. US2010/0272619A1 also does not describe the chemical-physical stabilization of atmospheric carbon in order to avoid a reaction with atmospheric oxygen to form (atmospheric) CO neither as a method nor as a device. US2010/0272619A1 certainly does not describe a material substitution of fossil CO₂s with atmospheric CO₂ or the incorporation of atmospheric carbon, which is chemically and physically stabilized, into agricultural soil in order to maintain or improve the humus content of this soil. US2010/0272619A1 does not at all describe the combination of its system with a fuel, heating medium or combustion material production and utilization system which combines the GHG-positive GHG emission quantities of a fuel, heating medium or combustion material determined according to LCA with the GHG-negative emission quantities of another compatible fuel, heating medium or combustion material also determined according to the LCA method to form a balanced zero quantity of a corresponding fuel, heating medium or combustion material mixture.

DE102004054468A1 (Lehmann), US 2006/275895A1 (Jensen & Jensen), the inventor's DE1020071029700A1/EP2167631A1 (Feldmann) and DE1020121 12898/EP13807989.2 (Lüdtke et al.) all disclose devices and methods for the anaerobic bacterial fermentation of straw. None of these published patent applications knows the difference between fossil and atmospheric carbon and fossil and atmospheric CO₂. None of these documents mentions a chemical-physical stabilization of atmospheric carbon in order to avoid a reaction with atmospheric oxygen to form (atmospheric) CO₂ neither as a method nor as a device, certainly not the chemical-physical stabilization of atmospheric carbon that might still be contained in the fermentation residues. These documents certainly do not disclose the incorporation of atmospheric carbon, which is chemically and physically stabilized, into agricultural soil in order to maintain or improve the humus content of this soil. These documents do not describe at all the combination of such a method or system with a fuel, heating medium or combustion material production and utilization system, which combines the GHG positive GHG emission quantities of a fuel, heating medium or combustion material determined according to LCA with GHG negative emission quantities of another compatible fuel, heating medium or combustion material also determined according to the LCA method to form a balanced zero quantity of a corresponding fuel, heating medium or combustion material mixture.

Also previously known is the inventor's EP07846568.9 (Feldmann), which discloses a biogas plant and a method for producing biogas from straw, in which the fermentation residues from anaerobic bacterial fermentation are pressed into fuel pellets or fuel briquettes after the dehydration. This document also does not know the difference between fossil and atmospheric carbon and fossil and atmospheric CO₂. Nor does it mention any chemical-physical stabilization of atmospheric carbon, which might possibly still be contained in the fermentation residues, in order to avoid a reaction with atmospheric oxygen to form (atmospheric) CO₂, neither as a method nor as a device. Certainly, EP07846568.9 does not disclose the incorporation of atmospheric carbon, which is chemically and physically stabilized, into agricultural soils in order to maintain or improve the humus content of these soils. EP07846568.9 does not at all describe the combination of such a method or system with a fuel, heating medium or combustion material production and utilization system which combines the GHG-positive GHG emission quantities of a fuel, heating medium or combustion material determined according to LCA with GHG-negative emission quantities of another compatible fuel, heating medium or combustion material also determined according to the LCA method to form a balanced zero quantity of a corresponding fuel, heating medium or combustion material mixture.

Furthermore, US2008/153145A1 (Harper) is previously known. This document teaches a method and a system for the disposal of farm manure, in particular the conversion of dairy cow excrements into ethanol, methane, carbon dioxide and fertilizers. The US2008/153145A1 system essentially consists of a collection hopper for cow excrement, a fermentation reactor with “methane outlet”, a methane drying and compaction unit, a methane pressure tank, a distillation column, storage tanks for ethanol and CO₂ and of a rotary kiln for the production of fertilizer granulate. The input material is exclusively dairy cow slurry. Milk cow slurry consisting of excrement, urine and water is collected in the collection bunker. When the slurry is transferred to the anaerobically working fermentation reactor, weakly acidic solution is added as a pretreatment measure in order to accelerate the first step of anaerobic fermentation, the hydrolysis of the cattle feed, which is only partially digested by the cows and excreted again (including cereal grain, cereal whole plant silage, hay and legumes). After one hour of hydrolysis, a basic substance is added to neutralize the acidified hydrolysis mass. After the neutralization, the hydrolyzed slurry is added to the fermentation reactor while adding a weak aqueous sugar solution (0.01%) and microorganisms of the species “Saccharomyces cerevisiae”. The anaerobic fermentation carried out by these microorganisms is said to produce ethanol and carbon dioxide, which are said to remain dissolved in the water. In addition, the microorganisms contained in the slurry also produce ethanol, carbon dioxide and methane. This ethanol and this carbon dioxide also allegedly remain dissolved in the water. The methane formed in the fermentation reactor rises and forms its atmosphere above the waterline. The allegedly pure methane is fed to the methane drying and compression unit via the outlet of the fermentation reactor, if necessary supported by a methane vacuum pump, and then to a compressed gas tank. US2008/153145A1 mentions as the use for the methane a use, sale and electricity generation for unspecified applications. The carbon dioxide dissolved in the water of the fermentation liquid and the ethanol also dissolved therein are separated from the water by a distillation column and pumped into the CO₂ pressure tank and the ethanol tank, respectively. US2008/153145A1 teaches as the use of the produced carbon dioxide how to “press” it into dry ice and sell it, and how to use the produced ethanol as a fuel.

Apart from the fact that the anaerobic bacterial fermentation does not produce pure methane, but—as is generally known—a gas mixture consisting of methane, carbon doxide, H₂S, hydrogen, nitrogen, ammonia and other gases, which is called biogas, the anaerobic bacterial fermentation also—as is also generally known—does not produce ethanol. US2008/153145A1 could therefore not work as intended. US2008/153145A1 distinguishes neither between atmospheric and fossil carbon nor between atmospheric and fossil CO₂. Due to the use of milk cattle slurry only atmospheric carbon comes back into the atmosphere of the earth and US2008/153145A1 also names the GHG effect of the gases methane and carbon doxide, but does not take any measures to remove these greenhouse gases or the underlying carbon from the atmosphere of the earth. The inventor of US2008/153145A1 is rather subject to the erroneous fallacy that the sale of this CO₂ or its “pressing” to dry ice or the use of methane to generate electricity removes the (atmospheric) carbon from the atmosphere of the earth—which is not the case (see introductory observations). The US2008/153145A1 method and system are therefore not suitable for reducing the CO₂ content of the atmosphere of the earth; a negative CO₂ content or the removal of (atmospheric) CO₂s from the atmosphere of the earth is a mandatory prerequisite for the (quantitative) compensation of positive (fossil) CO₂ emissions into the atmosphere of the earth. US2008/153145A1 also does not describe the chemical-physical stabilization of atmospheric carbon to avoid a reaction with atmospheric oxygen to (atmospheric) CO₂ neither as a method nor as a device. US2008/153145A1 certainly does not describe a material replacement of fossil CO₂s by atmospheric CO₂ or the incorporation of atmospheric carbon, which is chemically and physically stabilized, into agricultural soil in order to maintain or improve the humus content of this soil. US2008/153145A1 does not even describe the combination of its system with a fuel, heating medium or combustion material production and utilization system which combines the GHG-positive GHG emission quantities of a fuel, heating medium or combustion material determined according to LCA with GHG-negative emission quantities of another compatible fuel, heating medium or combustion material also determined according to the LCA method to form a balanced zero quantity of a corresponding fuel, heating medium or combustion material mixture.

Also previously known is WO2010/043799A2 (Morin), which describes a method and system for extracting carbon dioxide from the atmosphere of the earth. The system essentially consists of a high-temperature biomass dryer, a carbonization reactor, a thermochemical converter consisting of a combustion chamber and an oxidation chamber connected to it, a gas reformer and a plant for the production of synthetic carbon compounds. The input material used is biomass which, due to photosynthesis, represents a natural intermediate storage of atmospheric CO₂s. The comminuted biomass which has a water content of 40% to 55%, is dried in a high-temperature biomass dryer with 400° C. to 600° C. hot, low-oxygen exhaust air from the oxidation chamber to a residual moisture content of 10% to 15%. The exhaust air of the oxidation chamber, which is not specified in more detail and accounts for more than 70% of the exhaust gases of the entire plant, is released from the biomass dryer into the atmosphere after the drying process together with the water extracted from the biomass. In the pyrolysis reactor, the dried biomass is converted into a carbonization gas at 700° C. to 1,000° C. by using additional oxygen in a solid bed which comes from the oxidation chamber, has a temperature of 400° C. to 800° C. and consists of metal oxide. In the gas reformer, the material composition of the carbonization gas coming from the carbonization reactor is “adapted”, namely at 1,200° C. to 1,400° C. by oxygen-assisted combustion of the substances, i.e. tar and methane, contained in the carbonization gas. The carbonization gas, which essentially consists of carbon monoxide and water vapor and may be “adapted”, is then reformed into hydrogen and carbon dioxide in this gas reformer by means of a CO shift reaction. This mixed gas is supplied to chemical plants that use it to produce synthetic fuels, such as methanol or DME. The carbonization residue resulting from the carbonization of the biomass (carbon-containing coke ash) is mixed with a metal oxide circulating in the thermochemical cycle, fed into the combustion reactor where it is combusted while feeding a circulating flow bed material consisting of metal oxide and CO₂ produced internally in the process as well as CO₂ produced externally in the process. The oxygen required for the combustion of the carbonization residue originates from the oxidized metal flux bed material, which undergoes a reduction reaction in the combustion reactor and an oxidation in the oxidation chamber and which is continuously exchanged in a circuit between the combustion reactor and the oxidation reactor. The CO₂ produced in the combustion reactor during the combustion of the carbonization residue is cooled, dusted, dewatered and then divided into 3 partial streams: a CO₂ partial stream 1 is compressed for removal and, after appropriate transport, stored in ground water-containing soil layers; a CO₂ partial stream 2 is fed into the carbonization reactor to control or regulate the reactions taking place therein and a CO₂ partial stream 3 is fed back into the combustion reactor to control the reactions taking place therein. Oxygen is fed into the oxidation chamber to ensure the oxidation of the highly reactive metal flow bed material. The 400° C. to 600° C. hot exhaust gas from the oxidation chamber allegedly consists of low-air (certainly meaning low oxygen) exhaust air, which is fed into the biomass dryer.

In principle, the CO₂ content of the atmosphere is only reduced if the CO₂ is really permanently removed from the atmosphere of the earth. However, this is not the case with WO2010/043799A2. Apart from the fact that the storage of CO₂ in groundwater-bearing soil layers (aquifers) pollutes (acidifies) the groundwater in an environmentally unacceptable way, as is the case with the fracking method, this CO₂ storage is not permanent. As WO2010/043799A2 itself states, the groundwater can return to the surface of the earth after a relatively short time and release the CO₂ dissolved in the water there again—thus rendering the entire CO₂ sequestration invalid. Furthermore, the conversion of atmospheric carbon-containing CO₂s into energy carriers or carbon-containing substances alone is not enough. The conversion of atmospheric carbon or CO₂s, e.g. into electrical energy or into a fuel, does not remove the carbon from the atmosphere because when the energy carrier or fuel produced in accordance with WO2010/043799A2 is used, the carbon is burned with atmospheric oxygen, which produces CO₂—and the CO₂ thus returns to the atmosphere. Disregarding the CO₂ emissions at the (carbon) use stage shows that WO2010/043799A2 does not use the LCA method for the greenhouse gas analysis or GHG quantity determination. Apart from the fact that the CO₂ sequestration taught by WO2010/043799A2 is not really permanent, after using the energy carriers produced according to WO2010/043799A2, only a very small part of the carbon in the biomass or the CO₂s produced therefrom would be reach the final deposal site, namely the CO₂ partial stream 1 resulting from the combustion of the carbonization residue. This mini storage is already needed to compensate the GHG emissions of the energy input, which cannot be covered internally in the process, including the energy input for the production of the required oxygen. Consequently, neither the process nor the WO2010/043799A2 system is GHG negative.

Nor does WO2010/043799A2 describe the chemical-physical stabilization of atmospheric carbon to avoid reaction with atmospheric oxygen to form (atmospheric) CO₂ neither as a method nor as a device. WO2010/043799A2 certainly does not describe a material replacement of fossil CO₂s with atmospheric CO₂ or the incorporation of atmospheric carbon, which is chemically and physically stabilized, into agricultural soil in order to maintain or improve the humus content of this soil. WO2010/043799A2 does not at all describe the combination of its system with a fuel, heating medium or combustion material production and utilization system that combines the GHG-positive GHG emission quantities of a fuel, heating medium or combustion material determined according to LCA with GHG-negative emission quantities of another compatible fuel, heating medium or combustion material also determined according to the LCA method to form a balanced zero quantity of a corresponding fuel, heating medium or combustion material mixture.

Also previously known is the inventor's DE102010017818.7/EP2536839A1/WO2011101137A1-A8 (Feldmann), which reveals a method and plant for the production of CBM (compressed bio-methane) as a greenhouse gas-free fuel. The method and the plant of the invention are used to produce GHG emission-reduced energy carriers (compressed bio-methane CBM) according to the anaerobic bacterial fermentation process. They include inter alla a module for the separation and recuperation of regenerative (atmospheric) CO₂. The separated and recuperated atmospheric CO₂ is a) either fed to a geological final deposal site or b) used as a substitute for fossil CO₂ or c) fed to a reforming plant where it is reformed to CH₄ and/or CH₃OH. The generated energy carriers are used as GHG emission-reduced fuels. The fermentation residues from the single-stage or multi-stage anaerobic bacterial fermentation either substitute mineral fertilizers or are processed into fertilizers or fertilizer components after separation into a rather solid and a rather liquid phase. The GHG emission reduction performance of the energy carriers produced can be so high that they become GHG negative. The produced, possibly GHG-negative bio-methane can be mixed with natural gas (CNG) to form a mixed gas, the GHG emission value of which is set via the added CNG content. This means that the mixed gas can assume both positive GHG emission values and negative GHG emission values. It is also possible to produce an exactly GHG-neutral mixed gas by mixing GHG-negative bio-methane and GHG-positive CNG, the GHG emission value of which is 0.0 gCO₂-eq/MJ or 0.0 gCO₂-eq/kWh_{Hi}. DE102010017818.7/EP2536839A1, which already knows and addresses the difference between fossil and atmospheric CO₂, does not yet describe atmospheric carbon. This published patent application also does not describe any chemical-physical stabilization of atmospheric carbon still contained in fermentation residues in order to avoid a reaction with atmospheric oxygen to form (atmospheric) CO₂, neither as a method nor as a device. DE102010017818.7/EP2536839A1 certainly does not disclose the incorporation of atmospheric carbon, which is chemically and physically stabilized, into agricultural soil in order to maintain or improve the humus content of this soil. DE102010017818.7/EP2536839A1 does not at all describe the combination of such a method or system with a fuel, heating medium or combustion material production and utilization system which combines the GHG-positive GHG emission quantities of a fuel, heating medium or combustion material determined according to LCA with GHG-negative emission quantities of another compatible fuel, heating medium or combustion material also determined according to the LCA method to form a balanced zero quantity of a corresponding fuel, heating medium or combustion material mixture.

Also previously known is the inventor's DE102011051250/EP2724081 (Feldmann), which describes the production of pyrolysis gas using gasification plants, of biogas using biogas plants and of flue gas using combustion plants, with separation plants separating atmospheric CO₂ from these gases. The atmospheric CO₂ is recuperated and, after intermediate storage, is passed to a geological final deposal site (sequestration) or to an industrial plant for the substitution of fossil CO₂s or to a plant for the production of synthetic fuels such as syn-methane, methanol, ethanol, octane, butane or propane. The resulting absolute GHG quantity reductions in the stock of the atmosphere of the earth and the GHG additional quantities produced by (fossil) fuel consumption are combined to form a total GHG quantity, wherein the resulting LCA-GHG emission value of the at least one produced fuel, determined in accordance with IPCC (LCA) and measured in the technical unit gCO₂ equivalent/MJ or gCO₂ equivalent/kWh_{Hi}, is relieved by at least 5%, preferably by at least 50%, more preferably by at least 85% and in particular by 100%.

DE102011051250/EP2724081, which already knows and addresses the difference between fossil and atmospheric CO₂, does not yet describe atmospheric carbon. Nor does this published patent application describe any chemical-physical stabilization of atmospheric carbon still contained in fermentation residues in order to avoid a reaction with atmospheric oxygen to form (atmospheric) CO₂, either as a method or as a device. Certainly, DE102011051250/EP2724081 does not disclose the incorporation of atmospheric carbon, which is chemically and physically stabilized, into agricultural soil in order to maintain or improve the humus content of this soil. DE102011051250/EP2724081 does not even describe the combination of such a method or system with a fuel, heating medium or combustion material production and utilization system which combines the GHG-positive GHG emission quantities of a fuel, heating medium or combustion material determined according to LCA with the GHG-negative emission quantities of another compatible fuel, heating medium or combustion material also determined according to the LCA method to form a balanced zero quantity of a corresponding fuel, heating medium or combustion material mixture.

There is thus no method or system for converting biomass into marketable fuels, heating mediums or combustion materials that derive their greenhouse gas reduction performance from the fact that they chemically and physically stabilize atmospheric carbon still contained in the conversion residues and thus prevent the reaction with atmospheric oxygen—thereby reducing the GHG emission values of the energy carriers produced. There are certainly no methods and systems for producing fuels, heating mediums or combustion materials, the GHG emission reduction performance of which is so high due to the stabilization of atmospheric carbon that the amount of GHG in the atmosphere of the earth (the GHG level) is reduced. Furthermore, the combination of biogenic energy carrier production with atmospheric carbon stabilization and atmospheric carbon sequestration is unknown. Furthermore, there is no method and no system for the fuel, heating medium or combustion material production that uses chemically and physically stabilized carbon to maintain or improve the quality of agricultural or forestry soils.

### 4. PROBLEM

It is therefore the object of the invention to provide a method and a system that improve the GHG emission reduction performance of existing methods and systems for producing fuels, heating mediums or combustion materials, in particular by improving them in such a way that the GHG emission values of the fuels, heating mediums or combustion materials produced become negative—i.e. the GHG level in the atmosphere of the earth becomes smaller despite the use of the energy carriers produced.

From the energy producer's point of view, the incorporation of crop residues, especially straw, into the arable soil is useless rotting in the field. From the point of view of the farmer and the proven experts in agricultural science, it is absolutely essential for the legally anchored maintenance of the humus content and soil quality that a certain minimum proportion of the straw growth remains on the fields. Since the latter view has ethical, legal and political priority, the new straw growth existing every year with a share of about ⅓ can only be used to a very limited extent for energy utilization. This means that straw growth is only available to a very limited extent for the production of fuel or heating medium or combustion material. None of the previously known processes and none of the previously known plants, devices and systems can make the ⅔ of the straw growth, which on average must remain there to maintain the humus content of the agricultural land, accessible for energetic use.

It is therefore a further object of the invention to bring together the different specialist areas of the production of fuels heating mediums and combustion materials and agriculture in a new cross-over technology and to provide a method and a system with which, firstly, at least one GHG-negative biogenic energy carrier can be produced which, with a compatible GHG-positive energy carrier, can be converted into a GHG-neutral mixed fuel (or mixed heating medium, mixed combustion material) and with which, secondly, at least a part of the ⅔ of the straw growth, which so far could not be used energetically, is made accessible for energetic use and which thereby fulfil the farmer's objective of maintaining or even further improving the soil quality of his arable land and fields, in particular the humus content of his arable land and fields, despite the extraction or removal of straw.

### 5. SOLUTION AND ADVANTAGES

In order to solve this problem, the invention provides a method according to claim 1 and a system according to claim 27. Advantageous further developments are disclosed in the dependent claims and in this description. The wording of all claims is made part of the description by way of reference.

When reference is made to the prior art below, this should also include the technology which has been used in practice and is still being used, if applicable, as a process or method and/or as a device or system.

The present invention relates to a method and a system for improving the GHG emission reduction performance of fuels, heating mediums and combustion materials and for enriching agricultural land with C-containing humus, wherein these objects can preferably be achieved simultaneously but do not have to be achieved simultaneously.

The GHG emission reduction performance of the fuels, heating mediums or combustion materials is so high that not only can a significant GHG emission reduction be achieved compared to the fossil reference but the GHG balance or the GHG emission quantity of the generated fuel, heating medium or combustion material can even become negative, i.e. after the production, supply and use of the fuel, heating medium or combustion material, the greenhouse gas quantity in the atmosphere of the earth is lower than before.

The effect of the decarbonization of the atmosphere of the earth, which can lead to a GHG negativity (or to a negative GHG emission quantity), is achieved, according to the invention in that first of all carbon-containing residues of a first biomass conversion are converted by means of a chemical-physical treatment, such as carbonization (selection from pyrolysis, carbonization, torrefaction, hydrothermal carbonization HTC, vapothermal carbonization, gasification and any combination of these treatment methods) into biochar or vegetable coal or biocoke and atmospheric carbon contained in the residues of the biomass conversion is stabilized chemically and physically in such a way that under normal circumstances—not including combustion—it does not react or hardly reacts at all with other substances, in particular not with (atmospheric) oxygen.

If in the following no other information is given on the parameters, i.e. heating, reaction temperature, oxygen supply and reaction pressure, “HTC” is to be understood as the thermochemical conversion of an aqueous suspension containing biomass under oxygen deficiency and at a pressure of >1.2 bar and a reaction temperature of more than 150° C. and less than 350° C., wherein products of the HTC are process water and HTC coal.

Accordingly, “torrefication” means the thermochemical conversion of biomass under oxygen deficiency at a reaction temperature of more than 150° C. and less than 350° C., the product of torrefication being torrefied biomass. “Pyrolysis” is accordingly understood to mean the thermochemical conversion of biomass by oxygen deficiency at a reaction temperature of more than 300° C. to 1000° C., products of pyrolysis being combustible gas, biochar/plant carbon and oils. If in the following we speak only of “pyrolysis”, then low-temperature pyrolysis and high-temperature pyrolysis should also be included, unless something else results from the respective context. “Gasification” is understood to mean the thermochemical conversion of biomass by moderate to no oxygen deficiency at a reaction temperature of more than 500° C. to 1200° C., wherein products of gasification are combustible gas and biochar/vegetable coal. “Combustion” is understood to mean the thermochemical conversion of biomass by excess oxygen at a reaction temperature of more than 650° C. to 1600° C., wherein products of combustion are hot exhaust gas (flue gas) and ash.

The chemical-physical stabilization in particular inhibits or severely limits the natural reactivity of atmospheric carbon with (atmospheric) oxygen, i.e. CO₂ can no longer be produced except by combustion. If the biochar/vegetable coal or the biocoke is (stored) wherever it is protected from aggressive conditions, carbon is removed from the atmosphere of the earth—a (desired) decarbonization of the atmosphere of the earth occurs.

Preferably, the produced biochar/vegetable coal or biocoke is placed in soils (agricultural or forestry soils, deserts, permafrost soils, scree fields, etc.), water masses (oceans, lakes, aquifers) or in abandoned quarries, caverns or mines, simply stored in buildings protected from the weather or incorporated into (arable) soil. Due to the chemical-physical stabilization of the atmospheric carbon and the resulting degradation resistance, the atmospheric carbon contained in the biochar/plant carbon or in the biocoke remains there permanently after incorporation into the soil, preferably after incorporation into the field topsoil, and especially increases the stable permanent humus pool there.

Straw left in the field is not suitable for this purpose. Within a maximum period of 30 years it rots in an aerobic oxidation process and/or, like other organic primary substance (OPS) introduced into the soil or left there, is decomposed by the soil organisms into CO₂ and H₂O. This also applies to wood that is introduced into the soil. The degradation processes are digressive, i.e. initially the degradation rates or the degradation effects are high. In the course of time they decrease, but very soon there is not much left of the straw or wood worked into the soil. While straw left on the field and the atmospheric carbon contained in it are no longer present in the topsoil after about 30 years, this is not the case when the method according to the invention is used. Despite partial conversion into fuel, heating medium or combustion material, some of the atmospheric carbon bound in the straw can reach the soil in a stabilized form and remain there for hundreds or thousands of years. This increases the amount of humus in the soil, in particular the amount of stable permanent humus. The straw removal according to the invention and the straw treatment according to the invention thus lead to a better final condition in terms of soil quality than the retention of the straw growth on the fields.

Secondly, the effect of decarbonization, which can lead to GHG negativity (or a negative GHG emission quantity), is achieved by introducing stabilized, only partially stabilized or unstabilized atmospheric carbon in the form of biochar/vegetable coal/biocoke, native straw or straw-containing fermentation residues into deeper soil layers where there is neither soil respiration nor aerobic decomposition. This means that neither soil organisms nor atmospheric oxygen attack the carbon introduced. In this case, stabilization of the atmospheric carbon by pyrolysis or torrefaction/HTC/vapothermal carbonization, etc., is not absolutely necessary. A preferred embodiment of the method according to the invention can therefore be to introduce a selection of native straw, straw-containing fermentation residues, wood, partially stabilized biochar/vegetable coal and unstabilized biochar/vegetable coal and any combination of these substances into deeper soil layers where there is neither soil respiration nor aerobic rotting.

Since the carbon of the biomass was part of the atmosphere of the earth before its storage by photosynthesis in the plant biomass in the form of CO₂ and thus represents atmospheric carbon, the permanent (final) deposal of this atmospheric carbon corresponds to sequestration, i.e. the atmospheric carbon is permanently removed from the atmosphere of the earth. This decarbonization of the atmosphere of the earth is attributed to the product of the process, i.e. the energy carrier produced (fuel, heating medium, combustion material). The GHG-negativity (negative GHG-emission quantity) of the generated energy carrier results from the fact that the negative effect of the decarbonization described above (converted into CO₂ equivalents) is (clearly) greater than the sum of all positive GHG-effects (converted into CO₂ equivalents) of the production, supply and use of the fuel, heating medium or combustion material. In other words, after the production, provision and use of the energy carrier (fuel, heating medium or combustion material) there is a smaller amount of greenhouse gas in the atmosphere of the earth than before.

In this respect, a method in which at least a proportion of the biochar/vegetable coal/biocoke containing atmospheric carbon is sequestered (finally deposed) in an additional method step to the basic method of claim 1 in soil (geological formations), in stagnant waters, in the ocean or in aquifers, preferably in soils used for agriculture or forestry, more preferably in soils not used, or no longer used, for agriculture or forestry and in particular in peatlands, desert soils or permafrost soils, represents an advantageous variant of the method according to the invention.

If the chemically and physically stabilized or partially stabilized biochar or vegetable coal or biocoke is introduced into soils used for agricultural purposes and certain ancillary conditions are observed (inter alia loading of activated coal-like biochar/vegetable coal or biocoke with nutrients), the soil quality is improved, in particular the humus content and C-containing humus content of these soils increase, above all the C-containing humus content in the stable permanent humus pool. At the same time, a permanent sequestration of chemically stabilized atmospheric carbon takes place. Permanent sequestration of chemically stabilized atmospheric carbon prevents the carbon from reacting with (atmospheric) oxygen to form CO₂ or—as in rice fields—being converted anaerobically by soil organisms to form methane (CH₄). This means that neither CO₂ nor CH₄ can escape from the soil into the atmosphere of the earth. Due to the molar fraction of carbon C in the carbon dioxide molecule CO₂ of 12.0107/44.01=27.291%, sequestration of chemically stabilized atmospheric carbon prevents the emission of a CO₂ mass that is greater by the factor of 3.664 (1/27.291%) than the mass of sequestered carbon. The sequestration of 1 ton of stabilized atmospheric carbon thus prevents the formation of 3.664 tons of CO₂.

Due to the molar fraction of carbon C in the methane molecule CH₄ of 12.0107/16.043=74.866%, sequestration of chemically stabilized atmospheric carbon prevents the emission of a CH₄ mass that is greater by the factor of 1.336 (1/74.866%) than the mass of sequestered carbon, i.e. 1.336 tons of CH₄ per 1 ton of carbon C. Since the GHG effect of the greenhouse gas methane is known to be 25 times higher than that of the greenhouse gas carbon dioxide the non-emission of 1.336 tons of methane corresponds to the non-emission of 33.4 tons of CO₂.

In a preferred embodiment of the invention, not only stabilized biochar/vegetable coal/biocoke can be applied to the soil, but also a first mixture of stabilized and less stabilized (partially stabilized) biochar/vegetable coal/biocoke and a second mixture of stabilized and not at all stabilized biochar/vegetable coal/biocoke. Furthermore, a third four-part mixture can be applied, which consists of a) stabilized biochar/vegetable coal/biocoke and b) partially stabilized biochar/vegetable coal/biocoke, c) unstabilized biochar/vegetable coal/biocoke and/or d) untreated fermentation residues. The proportions of the four components of this mixture can each be 0%-100% under the obvious secondary condition that the sum of the four components does not exceed 100%.

The stabilization of the biochar/vegetable coal/biocoke (more precisely: of the atmospheric carbon contained in the biochar/vegetable coal/biocoke) is achieved according to the invention in that the carbon-containing residues from a first biomass conversion (anaerobic bacterial fermentation to biogas, fermentation to ethanol, transesterification to bio-diesel or bio-kerosene, gasification and synthesis to synthetic diesel or synthetic gasoline or synthetic kerosene or synthetic methanol, methanol synthesis, DME synthesis, etc.) are subjected to carbonization under oxygen deficiency at a temperature of 100° C.-1,600° C., preferably at a temperature of 200° C.-1,200° C., in particular at 300° C.-1,000° C. and in the best case at 400° C.-900° C. During carbonization, easily decomposable compounds from macropores and micropores are first dissolved (gasified) while a rigid framework consisting of carbon and polyaromatic carbon compounds with its more stable and less degradable structures is retained. The conversion residues used are thus converted into combustible gases and carbon-containing biochar/vegetable coal or into biocoke.

Preferably, the loss of atmospheric carbon resulting from the at least partial chemical-physical stabilization of the conversion residues is a maximum of 95%, preferably a maximum of 60%, in particular a maximum of 40% and at best a maximum of 30%.

Preferably, the dry substance loss occurring during the carbonization of the conversion residues from the single-stage or multi-stage biomass conversion is a maximum of 95%, more preferably a maximum of 60%, in particular a maximum of 40% and at best a maximum of 30%.

In an advantageous embodiment, the carbon content of the produced biochar/vegetable coal or the produced biocoke is at least 20%, preferably at least 40%, more preferably at least 60%, in particular at least 70% and in the best case at least 80%.

Other advantageous embodiments include the molar H/C ratio of a) the (partially) stabilized atmospheric carbon produced according to the method of the invention, b) the produced biochar/vegetable coals C to E with a high C content, c) the biochar/vegetable coal mixture H and/or d) the biochar/vegetable coal conversion residue mixture I at <0.8, preferably at <0.7 and more preferably at <0.6, and/or the molar O/C ratio thereof at <0.8 and preferably at <0.6 and more preferably at <0.4.

In a preferred embodiment of the invention, the heating and carbonization of the residues from the first biomass conversion take place according to any selection from the following reaction parameters: heating relatively slow, reaction time relatively long, reaction temperature relatively high, reaction pressure relatively high. The slower the heating of the conversion residues is carried out, the longer the conversion time, the higher the reaction temperature and the higher the reaction pressure, the more stable the produced biochar/vegetable coal or the produced biocoke becomes against chemical reactions and against degradation by soil organisms. It is therefore advantageous to heat the biomass added only slowly and to carry out a so-called high-temperature carbonization, if necessary, particularly slowly and/or even under pressure.

Preferably the heating of the mass to be carbonized to reaction temperature therefore takes longer than 1 second, more preferably longer than 10 minutes and in particular longer than 100 minutes. Preferably, the reaction mass is exposed to the reaction temperature for more than 1 second, more preferably for more than 50 minutes and in particular for more than 500 minutes. The reaction temperature is preferably more than 150° C., more preferably more than 300° C. and in particular more than 600° C. Preferably, the pressure in the reaction vessel corresponds to the pressure of the environment, more preferably >1 bar and in particular >5 bar and in the best case >10 bar.

The carbonization is preferably carried out in the form of pyrolysis. The drier the reaction mass, the better or more effective is the pyrolysis. The residues to be pyrolyzed from the first biomass conversion therefore preferably have a dry substance content (DS content) of at least 35%, more preferably at least 50% DS and especially at least 60% DS.

Due to their very porous outer and inner surface, which is much larger in the case of pyrolysis coals than in the case of HTC coal, pyrolysis coals have a high water absorption capacity, which in the case of incorporation into the soil means that the soil can store water better after the application of pyrolysis coal. In addition to clay-rich loam soils, the water storage capacity, especially of sandy soils, increases significantly after the addition of biochar/vegetable coal/biocoke, and even for heavy clay soils there is an increase in the water available to plants when mixed with pyrolyzed straw coal. On the other hand, such an effect cannot be achieved with wood chip coal. Here, the substrate straw has advantages which are based on a different pore structure. The effects of the coal application of pyrolysis coal on the water storage capacity of sandy soils are particularly positive. In an advantageous embodiment of the invention, straw-containing residues are therefore preferably subjected to an initial biomass conversion of pyrolysis, more preferably a high-temperature pyrolysis, and the resulting straw coal is applied in particular to sandy soils.

The advantageous embodiment of the partial stabilization of the biochar/vegetable coals or the biocoke (more precisely: of the atmospheric carbon contained in the biochar/vegetable coals/biocoke) is achieved in that the carbon-containing residues from the first conversion (any form of biomass conversion, preferably the anaerobic bacterial fermentation to biogas or the fermentation to ethanol) are subjected to torrefaction or low-temperature pyrolysis under oxygen deficiency at 150° C.-450° C., preferably at 200° C.-400° C., in particular at 250° C.-300° C. (low-temperature pyrolysis is hereinafter referred to as pyrolysis in which the reaction temperature is less than 450° C.; high-temperature pyrolysis is that in which the reaction temperature is more than 600° C.). Although this also produces biochar/vegetable coal/biocoke, it is not as resistant to reaction and degradation as pyrolysis coal, which can be positive for the content of active nutrient humus contained in the soil.

In an advantageous alternative embodiment, the partial stabilization of the biochar/vegetable coals or the biocoke (more precisely: the atmospheric carbon contained in the biochar/vegetable coals/biocokes) is achieved by subjecting the carbon-containing residues from the first conversion to hydrothermal carbonization (HTC) in the presence of water or steam, under oxygen deficiency and under pressure. The temperature is 130° C.-400° C., preferably 150° C.-300° C. and in particular 180° C.-250° C. The pressure is here 1.2-200 bar, preferably 10-100 bar and in particular 20-50 bar. HTC products are so-called HTC coal and process water. Like torrefied biomass, HTC coal is not as resistant to reaction and degradation as pyrolysis coal, which can have a positive effect on the content of active nutrient humus in the soil.

Preferably, the method according to the invention and the system according to the invention produce pyrolysis coal. Compared to pyrolysis coal, HTC and torrefaction coal are degraded much faster in the soil, namely within a few decades. Pyrolysis coals have a relatively high proportion of complex polyaromatic carbon structures and are therefore much more stable than HTC coals which have a lower content of polyaromatic carbon compounds and a correspondingly higher content of easily mineralizable (degradable) C compounds. The stability of HTC and torrefaction coals is more similar to that of composts and peat. A long-term sequestration of (atmospheric) carbon is therefore not possible or only possible to a very limited extent with such coals.

Preferably, the molar H/C ratio of the biochar/vegetable coal or biocoke produced according to the method of the invention is <0.8, more preferably <0.6, and its molar O/C ratio is <0.8, more preferably <0.4. The molar H/C ratio indicates the degree of charring which correlates with the chemical stability of the biochar/vegetable coal or biocoke. This ratio is one of the most important properties of biochar/vegetable coal or biocoke. The H/C ratio should be below 0.8 to ensure sufficient degradation resistance for permanent sequestration. With the aging of the biochar/vegetable coals or the biocoke and the oxidation of their surfaces, the O/C and also H/C ratios gradually increase, so that it is desirable for a maximized sequestration effect if fresh coal or coke when introduced into the soil has both the lowest possible O/C ratio (<0.4) and a minimum H/C ratio (<0.6): both increase their residence time in the soil and thus the long-term sequestration effect.

If the biochar/vegetable coal or the biocoke, which consists mainly (5%-95%) of stabilized atmospheric carbon, is mixed with unstabilized or poorly stabilized biochar/vegetable coal/biocoke and/or with conversion residues that have not undergone any chemical-physical post-treatment, the labile pool of nutrient humus also receives sufficient organic primary substance OPS during incorporation into the soil so that the soil organisms are sufficiently provided with food and can continue to exist and the soil quality does not suffer. Since in the case of the use of straw-containing conversion residues during straw harvesting, a certain part of the straw growth in the form of stubble, husks and chaff always remains in the field and is incorporated into the field topsoil during the subsequent soil tillage, the basic supply of the unstable nutrient humus pool with organic primary substance (OPS) is ensured. The proportion of unstabilized or poorly stabilized biochar/vegetable coal/biocoke and/or conversion residues that have not undergone chemical-physical after-treatment can therefore generally be significantly lower than the proportion of stabilized biochar/vegetable coal/biocoke.

In an advantageous embodiment, the stream of conversion residues emerging from the method step of single-stage or multi-stage biomass conversion can therefore have up to 4 partial streams prior to thermochemical treatment, namely the first partial stream “pyrolysis coal”, the second partial stream “torrefaction coal”, the third partial stream “HTC coal” and the fourth partial stream “untreated conversion residues”. The partial streams can each have a share of 0% to 100% of the total stream and of the product produced, i.e. each partial stream can represent both the total stream and zero.

The introduction of stabilized and partially stabilized carbon into the agricultural soil gives the soil more carbon than if the straw had remained in the field and rotted there and/or if the straw was converted into CO₂ and water during soil respiration. Consequently, it is advantageous for the quality of the topsoil if the straw does not remain on the field but is removed and partly converted into fuel (heating medium, fuel) and partly into (partly) stabilized biochar/vegetable coal or biocoke, and this (partly) stabilized biochar/vegetable coal or (partly) stabilized biocoke is incorporated into the topsoil. The retention of the straw in the fields thus becomes superfluous if the method according to the invention is used. This gives the user of the method according to the invention at least partial access to the ⅔ of the straw that had to remain in the fields to maintain the humus content.

Biochar/vegetable coal/biocoke produced from straw reaches carbon contents of 25%-79% depending on the type of straw used, the charring process used, the type of equipment employed and the process parameters (temperature increase curve, maximum temperature, treatment duration, pressure). Therefore, carbonization processes, plants and/or process parameters are preferred which produce biochar/vegetable coal/biocoke with a relatively high carbon content, preferably biochar/vegetable coal/biocoke with a carbon content of >25%, more preferably biochar/vegetable coal/biocoke with a carbon content of >50% and in particular biochar/vegetable coal/bio coke with a carbon content of >70%.

In addition, biochar/vegetable coal/biocoke produced from straw at high temperatures has high pH values of up to 11.3, which predestines them for introduction into acid soils. Alkaline biochar/vegetable coals/biocokes, like most pyrolysis coals, have high pH values that increase the pH of acidic and weakly basic soils, resulting in an improvement in the mineralization of organic sulfur compounds, an improvement in other humus mineralization and an improvement in the microbial degradation of OBS. In addition, the application of basic pyrolysis coal in acidic soils results in an increase in the earthworm population. Preferably, the biochar/vegetable coal/biocoke is therefore produced at least in part from straw-containing conversion residues by means of high reaction temperatures. Preferably, the biochar/vegetable coal/biocoke produced from conversion residues containing straw has a pH value of >7.0, more preferably >8.5 and in particular >10.0. Preferably, the biochar/vegetable coal/biocoke produced from conversion residues containing straw is applied to acidic soils.

Biochar/vegetable coals/biocoke have a large capacity for sorption, binding and storage of nutrient ions as well as inorganic and organic compounds. This results from their very large inner and outer surface area, which is significantly larger for pyrolysis coal than for torrefaction and HTC coals. The recuperated residues from the first biomass conversion are therefore primarily subjected to pyrolysis, preferably >1% of the recuperated residues, more preferably >50% of the recuperated residues and in particular >75% of the recuperated residues from the first biomass conversion.

After the application of biochar/vegetable coal or fresh biocoke, in particular those produced at low temperatures by the HTC process, the effect of temporary nitrogen immobilization can occur. This effect has its reasons in the binding of the NH₄ ion and the resulting reduction in nitrification and in the increased soil respiration. Although these effects are usually only of a short-term nature, the proportion of HTC coal in the coal mixture consisting of several biochar/vegetable coal species or biocoke is therefore minimized to <99%, preferably to <50%, more preferably to <25% and in particular to <10%.

As the biochar/vegetable coal/biocoke ages, parts of the porous surfaces oxidize. The oxidation of the surfaces creates functional groups with a negative excess load. The sorption capacity for nutrient cations (e.g. K⁺, Mg²⁺, NH₄⁺) develops in the course of the aging of fresh biochar/vegetable coals or fresh biocoke or through special measures (e.g. activation with steam). Depending on the availability of nutrients, microbial colonization of the coal particles takes place. In addition, there is also a considerable storage and adsorption capacity of the biochar/vegetable coals/biocoke for nutrient anions (e.g. PO₄). For example, the nutrient availability of vegetable carbon phosphorus in the first year after application is about 15% and that of nitrogen only about 1%, while up to 50% of potassium in the first year is available to plants. According to the invention, the stabilized and partially stabilized biochar/vegetable coals or biocoke are therefore enriched with nutrients prior to application in soils, especially if the soils are used for agricultural purposes.

This enrichment with nutrients, which is preferably an enrichment with nitrogen compounds, more preferably an enrichment with organic nitrogen compounds, is also called loading. An enrichment of the biochar/vegetable coal/biocoke with nutrients, preferably with organic nutrients, prior to its introduction into the soil is advantageous because these occupy the extremely porous surface of the coal particles. Due to the nutrient enrichment the loading also leads to a rapid activation of the surface of the carbon skeleton by microbial colonization. The skeleton is thus covered with convertible organic materials that become part of the active nutrient humus, while the skeleton itself remains part of the passive permanent humus. Short-term negative effects on the nitrogen balance can thus be minimized. They are also overcompensated by subsequent positive effects.

Loading with (organic) nutrients prevents the activated carbon or charcoal effect that occurs when unloaded fresh biochar/vegetable coal/biocoke is introduced into the arable soil. Without loading, soil constituents, especially nutrients found in the soil, such as the various forms of nitrogen compounds, would accumulate on the very large, porous surface of the fresh coal particles introduced. The initial reduction in nitrogen availability after the introduction of fresh biochar/vegetable coal or fresh biocoke into the soil is due to its very large external and internal porosity and a high sorption capacity for cations, which allows the coal to adsorb NH₄ to a large extent and which also physically “traps” the NH₄ ion in the pores. With increasing age of the coals, a concomitant oxidation of the surfaces and the formation of functional chemical groups, this effect decreases, i.e. In the medium term the absorbed nitrogen becomes available for plants again. The loaded biochar/vegetable coal/biocoke can therefore preferably also be used as a fertilizer, more preferably as a long-term nitrogen fertilizer.

The loading of the stabilized, partially stabilized or unstabilized biochar/vegetable coal/biocoke can be carried out by quenching the hot and absolutely dry biochar/vegetable coal/biocoke coming from the pyrolysis with a nutrient-containing aqueous suspension, preferably with a selection from the aqueous suspensions, i.e. slurry, percolate, swill, urine, seepage water from silages, stillage from ethanol production, liquid residues from anaerobic fermentation, process water, treated or purified process water, liquid fermentation mass, permeate, more liquid phase of dehydration, more solid phase of dehydration, any phase of separation, suspensions containing other nutrients and similar suspensions. Preferably, only enough liquid is used to keep the quenched biochar/vegetable coal/biocoke dry. In this context, “dry” means that the quenched biochar/vegetable coal/biocoke does not release any free water after the quenching process. In particular, the suspension used to quench the hot biochar/vegetable coal/biocoke is the more liquid phase of dehydration of the residues from the single-stage or multi-stage biomass conversion, which takes place prior to pyrolysis or torrefaction.

The loading of the biochar/vegetable coals/biocoke with easily degradable organic substances and thus a nutrient enrichment of the biochar/vegetable coals/biocoke can also take place by composting them together with farm manure and/or straw (aerobically rotting).

In the course of coal aging, the biochar/vegetable coal/biocoke, consisting of carbon and very stable carbon compounds, reacts on its outer and partly also on its inner surfaces, acting both as a catalyst and as a reagent. Amino, phenol, hydroxyl, carbonyl or carboxyl groups are thus formed. At the same time, the negative charge of the surfaces increases, resulting in an increased cation exchange capacity. Therefore, biochar/vegetable coal/biocoke can absorb and bind nutrients and make them available to microorganisms, fungi and plants over a longer period of time. Due to their polarity, hydrophilic groups also lead to improved water storage. The loaded biochar/vegetable coal/biocoke can therefore preferably also be used as a soil improver.

The coal particles therefore release the nutrients only gradually and only over long periods of time, so that a negative fertilizing effect, which is usually unintentional, occurs in the short term when fresh biochar/vegetable coal or fresh biocoke is introduced. However, if this negative fertilizing effect is intended, e.g. In the case of a nitrogen surplus in the soil or in the case of nitrogen leaching from the (agriculturally used) soil into the groundwater, fresh biochar/vegetable coal/biocoke that has not been loaded with nutrients can also be introduced into the arable soil instead of loaded biochar/vegetable coal/biocoke. According to the invention, unloaded stabilized biochar/vegetable coals/biocoke can, therefore, also be incorporated into excessively fertilized and/or sandy soils in order to reduce excessive fertilization and/or nitrogen leaching.

With regard to nitrogen management, the incorporation of stabilized biochar/vegetable coal or stabilized biocoke into the arable soil initiates a cycle of self-reinforcing individual effects: In addition to nitrogen immobilization, the above-mentioned effects due to coal application (NH₄ sorption, N-immobilization, retention in pores) result in a reduction in nitrogen leaching with seepage water, which is particularly the case with sandy soils. However, N-leaching from the main root zone of corn crops also decreases significantly with the application of stabilized biochar/vegetable coal or stabilized biocoke. Since the cultivated plants also make better use of nitrogen fertilization, there is a further reduction in N-leaching. The effects of the increased activity of microorganisms, a significant increase in water storage capacity and an increased colonization of the roots with symbiosis fungi (mycorrhizae) in the treated soils associated with coal application also contribute to this. Thus, the administration of stabilized biochar/vegetable coal or stabilized biocoke improves the N-storage of the soils and at the same time reduces their nitrogen losses not only by direct adsorption but also by a significant reduction in N-leaching and by further secondary effects.

The introduction of stabilized biochar/vegetable coal or stabilized biocoke, in particular pyrolysis coal, into the soil thus allows an improvement in N efficiency and consequently a reduction in total nitrogen fertilization. Above all, the reduction in N-leaching contributes to the solution of a central problem of German agriculture. For example, the official nitrate limit value has been regularly exceeded in large parts of Schleswig-Holstein since 2005, especially on the geest ridge. For this reason alone, the innovative incorporation of stabilized biochar/vegetable coals or stabilized biocoke into the plant soil, which is not (yet) permitted by law, will become increasingly important, not least from an environmental point of view.

As long as the stabilized pyrolysis coals are not produced from input materials with relatively high N contents, as is the case, for example, with poultry manure, soils treated with pyrolysis coal consistently release less laughing gas (N₂O) into the atmosphere than untreated soils. Therefore, biomasses with relatively low N contents, such as straw and wood, are preferably used in the method according to the invention.

However, even the input material-related N₂O emissions which in some cases can initially be higher than in soils not treated with pyrolysis coal, usually decrease significantly after about 4 months, namely below the emission level of soils not treated with pyrolysis coal. The reduction in N₂O emissions is greater for fresh pyrolysis coal than for aged pyrolysis coal. Reasons are the decrease in the mineral N-contents, the higher pH values of the pyrolysis coal and the resulting better conditions for N₂O-degrading enzymes as well as the reduction in the denitrification by increased soil aeration and increasing N-immobilization. The pyrolyzed biochar/vegetable coals/biocoke produced according to the method of the invention can therefore also be used as soil conditioners.

The large external and internal surface area of the biochar/vegetable coals/biocoke, above all of the pyrolysis coals, and the negative surface charge increasing with coal age have the effect—as already explained—that there is an increase in the capacity of the treated soils to exchange cations. In addition to increasing the bioavailability of the important nutrient cations, i.e. Ca, Mg and Na, the increase in K-storage is particularly relevant for plant nutrition. The incorporation of biochar/vegetable coals/biocoke into the soil also has a positive effect on the plant availability of Mn and Cu. The bioavailability of micronutrients is also improved by the application of biochar/vegetable coals/biocoke. The biochar/vegetable coals/biocoke produced according to the method of the invention can therefore also be used as macro and micro fertilizers and in particular as potassium fertilizers.

The introduction of pyrolysis coals into cereal cultivation areas has the further positive effect that an intensified and beneficial colonization of wheat roots with symbiotic soil fungi (arbuscular mycorrhiza fungi—AMF) occurs. The biochar/vegetable coals/biocoke produced according to the method of the invention are therefore applied in a preferred embodiment of the invention as pyrolysis coals prior to the cultivation of cereal crops.

Preferably, the method according to the invention uses conversion residues containing wheat straw since a significant decrease in plant parasitic nematodes can be observed with the application of wheat straw-based pyrolysis coal.

Whether and to what extent the use of stabilized biochar/vegetable coal or stabilized biocoke has soil-improving effects in a specific individual case is determined not only by the starting materials and the methods for producing the biochar/vegetable coal/biocoke but also by the site-specific factors of soil genesis and mineralization which already determine the humification of OBS and the way in which the soil is used. A positive effect can be expected in particular if the administration of biochar/vegetable coal/biocoke improves one or more yield-limiting soil properties, such as too low humus content, excessively acid soil, too low nutrient availability, excessively high nutrient availability, too little water supply and too little microbial activity. The biochar/vegetable coal/biocoke provided by the method according to the invention and the system according to the invention is therefore preferably used to improve at least one of these yield limiting soil properties.

The effects of biochar/vegetable coals/biocoke on soil fauna and flora are a function of the properties of the coal used (starting material, production process, post-treatment, loading) and the chemical and physical properties of the site. The functional structure is very complex, changes in the chemical and physical soil properties by application of biochar/vegetable coals/biocoke influence the population densities of the soil organisms and thus the soil biological activity and these, in turn, the soil properties. For example, it can be assumed that microorganisms use pyrolysis coal only to a very limited extent, if at all, as a nutrient or energy source due to their chemical stability, and that microbial activity therefore does not increase immediately after the introduction of such coal into the soil. Surprisingly, however, it was found that after the use of pyrolysis coal produced at high temperatures, bacterial species and bacterial numbers increase. This is due to the outer and inner surfaces of the coal particles, which are particularly large in pyrolysis coal. The coal particles offer the microorganisms new habitats either alone or as part of a so-called humus aggregate and thus promote their growth. Therefore, above all pyrolysis coals are preferably produced with the method according to the invention and, after being loaded with nutrients, introduced into agricultural soils as a soil improver or fertilizer.

Last but not least, the use of biochar/vegetable coals/biocoke can increase the plant growth and crop yield. The yield-increasing effect of the application of biochar/vegetable coal/biocoke here depends on the amount of coal incorporated into the soil: The more “correct” biochar/vegetable coal/biocoke is used, the more likely a yield-increasing effect occurs, wherein there are (very high) upper limits for the application, beyond which opposite effects occur. Light, sandy and humus-poor locations require the use of 20-100 t biochar/vegetable coal/biocoke dry substance per hectare (ha). Experimental cultivation trials in greenhouses have shown that on sandy and loamy soils coal inputs of <3 t/ha do not lead to increases in rye yields. It has to be taken into account that coal applications generally only have to be carried out once every 100 years, while the application of fermentation residues and compost has to be carried out annually or every 3 years. For significant soil and yield-improving effects to occur, certain minimum quantities of biochar/vegetable coal/biocoke are required. Positive effects on plant growth are already observed with incorporation quantities of 15 t/ha. However, in order to achieve noticeable effects, much larger quantities of coal usually have to be incorporated into the soil. The biochar/vegetable coals/biocoke produced according to the method of the invention are therefore preferably used in quantities such that the yield of agricultural land increases. Preferably, at least 5 tons of biochar/vegetable coal/biocoke dry substance are applied per hectare, more preferably at least 20 tons, in particular at least 50 tons and in the best case at least 100 tons. These quantities can refer both to fully and partially stabilized atmospheric carbon and to the quantities of biochar/vegetable coal applied.

It should be taken into account that even applications of 100 tons of biochar/vegetable coal/biocoke per hectare represent only a relatively small application in relation to the soil mass: The average density of arable topsoil is 1.65 g/cm³, which means that in relation to one hectare and a depth of 30 cm it has a mass of about 5,000 tons (100m×100m×0.30m×1.65 t/m³=4,950 t). The incorporation of 25 t of biochar/vegetable coal/biocoke per hectare thus corresponds to a relative share of 0.50% of the soil mass and the administration of 50 t to a relative share of 1.00%. With the incorporation of 75 t of biochar/vegetable coal/biocoke, only a share of 1.50% is achieved and even 100 t increase the relative share of biochar/vegetable coal/biocoke in the soil mass to only 2.00%. Compared to the achievable peak values of up to 10%, these humus contents are still relatively low, wherein the humus C contents are even lower corresponding to the carbon content of the biochar/vegetable coal/biocoke.

Since the higher the temperature at which the coals are produced, the higher the yield-increasing effect, the biochars/vegetable coals/biocokes produced according to the methods of the invention are preferably produced according to the sub-method of high-temperature pyrolysis.

It is therefore clear that pyrolysis coals, in particular high-temperature pyrolysis coals, not only stabilize atmospheric carbon better, but that these coals are also better suited for application in arable soils than other biochars/vegetable coals/biocokes. Pyrolysis coal produced from straw provides special services in several respects (see above). Pyrolysis coal produced from straw at a relatively high temperature is not only suitable for incorporation into light, sandy arable soils but also into heavy soils and thus for long-term C sequestration. Therefore, in advantageous embodiments of the invention, straw or straw-containing conversion residues are used in particular.

Since the incorporation of straw-containing solid manure and straw-containing fermentation residues from anaerobic bacterial fermentation into the soil is practiced without problems already now, it should be possible in light of the above-mentioned invention to also incorporate straw-containing fermentation residues into the soil which have previously been subjected to high-temperature pyrolysis. At present, this is not (yet) possible in Germany, contrary to the regulations laid down by European fertilizer law. According to German law, charcoal made from untreated wood using pyrolysis is currently the only carrier material for nutrients that can be placed on the market. Before the German Soil Protection Act might be able to permit the use of biochar/vegetable coal/biocoke in agriculture, negative consequences for soil functions would have to be ruled out and corresponding criteria be developed for Germany. Negative effects regarding the introduction of biochars/vegetable coals/biocokes into the soil, such as the supply of pollutants (e.g. heavy metals) and increased release of substances that could endanger the air and water to be protected or lead to health risks for plants, animals and humans, can, however, be minimized by the selection of low-pollutant input materials, by appropriate process control and by appropriate after-treatments. Since straw and wood are relatively low-pollutant, straw, straw-containing residues and residues of unpolluted wood from an initial biomass conversion are used in the method according to the invention.

Thirdly, the effect of decarbonization or GHG emission reduction of the fuel produced (heating medium, combustion material), which preferably results in a GHG negativity (or negative GHG emission quantity), can be achieved by recuperating produced atmospheric CO₂ during the production of the biochar/vegetable coal/biocoke, which is composed of atmospheric carbon and atmospheric (air) oxygen, and replacing fossil CO₂ (e.g. In the beverage industry), which usually consists of fossil carbon. In order to produce carbon dioxide, also known as carbonic acid when dissolved in water, extra fossil natural gas (CNG) is burned worldwide. The substitution of this fossil carbonic acid by atmospheric CO₂ avoids the emission of fossil CO₂s, which relieves the atmosphere of the earth (additional sub-process Z1a). In a preferred embodiment, the atmospheric CO₂ produced during the production of fuel, heating medium or combustion material can therefore be recuperated and made available for industrial use.

The effect of decarbonization or GHG emission reduction of the fuel produced (heating medium, combustion material), which preferably results in a GHG negativity (or a negative GHG emission quantity), can be achieved in the fourth place by permanently sequestering the recuperated atmospheric CO₂ produced during the production of the fuel, heating medium or combustion material in geological strata (e.g. In depleting geological oil or gas deposits), which also removes atmospheric carbon from the atmosphere of the earth (additional sub-process Z1b). According to the invention, the recuperated carbon dioxide can therefore be liquefied and transported in this aggregate state to the geological oil or gas deposits.

The effect of decarbonization or GHG emission reduction of the fuel produced (heating medium, combustion material), which preferably results in a GHG negativity (or negative GHG emission quantity), can be achieved in the fifth place by using the recuperated atmospheric CO₂ produced during the production of the fuel, heating medium or combustion material in order to produce CO₂-based energy carriers, such as hydrogen gas produced by wind power via water electrolysis, which is converted into synthetic methane (syn-methane) according to Sabatier using CO₂ (additional sub-process Z1c).

According to the invention, the recuperated carbon dioxide can therefore be made available to corresponding manufacturing processes, preferably in a liquid aggregate state.

The effect of decarbonization or GHG emission reduction of the fuel produced (heating medium, combustion material), which preferably results in a GHG negativity (or in a negative GHG emission quantity), can be achieved in the sixth place by recuperating the atmospheric CO₂ produced during the chemical-physical stabilization of the atmospheric carbon still contained in the conversion residues and replacing fossil CO₂ (additional sub-process Z2a). According to the invention, the atmospheric CO₂ produced during the chemical-physical stabilization of the atmospheric carbon still contained in the conversion residues can therefore be recuperated and made available for industrial use.

The effect of decarbonization or GHG emission reduction of the fuel produced (heating medium, combustion material), which preferably results in a GHG negativity (or in a negative GHG emission quantity), can be achieved in the seventh place by recuperating the atmospheric CO₂, which is produced during the chemical-physical stabilization of the atmospheric carbon still contained in the conversion residues, and by sequestering it (additional sub-process Z2b). According to the invention, the atmospheric CO₂ produced during the chemical-physical stabilization of the atmospheric carbon still contained in the conversion residues can therefore be recuperated, liquefied and transported in this aggregate state to the geological oil or gas deposits or other sequestration sites (aquifers, oceans, lakes, etc.).

The effect of decarbonization or GHG emission reduction of the fuel produced (heating medium, combustion material), which preferably results in a GHG negativity (or a negative GHG emission quantity), can be achieved in the eighth place by recuperating and using the atmospheric CO₂ produced during the chemical-physical stabilization of the atmospheric carbon still contained in the conversion residues to produce CO₂-based energy carriers, such as hydrogen gas produced by wind power via water electrolysis, which is converted according to Sabatier into synthetic methane (syn-methane) using CO₂ (additional sub-process Z2c). According to the invention, the atmospheric CO₂ produced during the chemical-physical stabilization of the atmospheric carbon still contained in the conversion residues can therefore be recuperated and made available for corresponding manufacturing processes, preferably in a liquid state of aggregation.

Particularly advantageous GHG effects (high GHG negativity) occur when the stabilization of the carbon-containing residues from a first biomass conversion is combined with one of the sub-processes Z1a to Z1c. In this context, it should be noted that the chemical-physical stabilization of the atmospheric carbon is already sufficient to prevent the formation of (atmospheric) CO₂ from this carbon, a permanent sequestering of the stabilized carbon is not absolutely necessary.

Particularly advantageous GHG effects (high GHG negativity) also occur when the stabilization of the atmospheric carbon still contained in the residues from a first biomass conversion is combined with one of the Z2a to Z2c sub-processes.

Especially advantageous GHG effects (very high GHG negativity) occur when the stabilization of the atmospheric carbon still contained in the residues from a first biomass conversion is combined with one of the sub-processes Z1a to Z1c and one of the sub-processes Z2a to Z2c.

The GHG negativity (or the negative GHG emission quantity) of the fuel, heating medium or combustion material produced in this way makes it possible that a compatible GHG-positive fuel, heating medium or combustion material can be mixed with it at least in part, without the resulting GHG emission value of the mixed fuel (mixed heating medium, mixed combustion material) turning positive. This results in a considerable increase in the available quantity of fuel (heating medium, combustion material) which is at least GHG-neutral. The produced energy carrier (fuel, heating medium or combustion material) is therefore preferably mixed with a GHG positive energy carrier in such a way that the resulting energy carrier mixture has a GHG emission value of 0.0 gCO₂-eq/kWh_{Hi} or 0.0 gCO₂-eq/MJ.

An option for an initial conversion of biomass into a (marketable) energy carrier is the anaerobic bacterial fermentation of straw into biogas and the processing thereof into bio-methane. With a conversion efficiency of 70% in this first biomass conversion, up to 2,860 kWh_{Hi} of straw-gas are produced from a straw input of 1 ton of wet mass with a usual water content of 14% (dry substance content 86%). At the same time, the GHG emission value of the method according to the invention has a GHG value of up to −648 kg CO₂-eq despite the GHG emissions resulting from the various manufacturing steps.

With a specific life cycle THG emission value of 249.5 gCO₂-eq/kWh_{Hi} CNG, the anaerobically produced 2,860 kWh_{Hi} straw-gas can therefore be mixed with up to 648,000 gCO₂/249.5 gCO₂/kWh_{Hi} CNG=2,597 kWh_{Hi} CNG without the resulting mixed gas quantity of 5,457 kWh_{Hi} being converted into a positive GHG emission value. This means that the method according to the invention can produce an absolutely GHG-neutral amount of fuel (amount of heating medium, amount of combustion material) of up to 5,457 kWh_{Hi} from 1 ton of straw wet mass, which corresponds to the calorific value of 620 liters of gasoline. The proportion of admixed CNG is here 2,597/2,860=90.8% in relation to the fuel (heating medium, combustion material) produced directly from the straw.

It can be assumed that the annual growth of straw in Germany will increase from the current 43.7 million tons of wet mass to up to 46 million tons of wet mass in the future, as the increasing demand for straw will lead farmers to saw and harvest cereals with longer straws again. It can also be assumed that combine harvesting and collecting technology will also be improved in the future and that the recovery rate can therefore be increased from the current 72% to 87%. As a result of the invention disclosed here, the collectable amount of straw will increase by a factor of 1.6 from the current 24.8 million t straw-WM/a to about 40 million t WM/a. As a result of the latent increase in slurry stalls, the need for bedding has now fallen to around 4.15 million t straw-WM/a. Since the method according to the invention and the system according to the invention can guarantee the maintenance of the humus content of the soil with the application of stabilized vegetable coal loaded with plant nutrients, it is not necessary to leave further parts on the fields in addition to the unrecoverable part of the straw growth. After deduction of the bedding and roughage requirements at the current level of 4.15 million t/a, 35.85 million t straw/a remain for energy use. The method according to the invention and the system according to the invention thus increase the quantity of straw that can be used for energy purposes in Germany alone by a factor of 2.0-4.5 from the about 8.0-13.0 million t straw-WM/a determined by the GBRC to about 35.85 million t straw-WM/a.

With a production capacity of up to 5,457 kWh_{Hi} of mixed fuel per ton of wet straw mass (see above), this energetically usable straw removal alone for Germany results in an absolutely GHG-neutral fuel potential of up to 35.85 million t straw-WM×5,457 kWh_{Hi}/t straw-FWM=195,633 GWh_{Hi} (704 PJ). This corresponds to 30% of the calorific value of all fuels used in German road traffic in 2016 and is significantly more than the experts had previously assumed as the available quantity of fuel.

Currently, an average German passenger car powered by an Otto engine consumes about 6,145 kWh_{Hi} of gasoline per year (about 700 liters of gasoline-equivalent) and an average diesel passenger car about 11,500 kWh_{Hi} (about 1,160 liters) due to its significantly higher annual mileage. Due to further improved engine technology and increasing hybridization, the annual consumption of Otto passenger cars will drop to about 4,000 kWh_{Hi}/a in the future. If the German straw collection is fully utilized, the mixed gas volume of 195,633 GWh_{Hi} can supply a vehicle fleet of up to 48.9 million Otto cars, and in absolute GHG-neutral terms. If the current energy efficiency is doubled (annual energy consumption halved to about 3,000 kWh_{Hi}/Otto cars), the number of vehicles that can be supplied with GHG-neutral fuel can increase to up to 65 million Otto car-equivalents.

In Germany, annual quantities of farm manure (slurry, solid manure) and leaf waste (beet and potato leaves as well as legume waste) amount to about 191 million tons of wet mass or 21.7 million t dry mass. This dry mass contains about 10.0 million t atmospheric carbon. With the method and system according to the invention, another up to 68,600 GWh_{Hi} of fuel might be produced from this with an assumed conversion efficiency of 70%. In the case of anaerobic bacterial fermentation, the usually high nitrogen content of these input materials ensures, when mixed with the input material, i.e. straw, which has a low N content, a C:N ratio which meets the requirements of the microorganisms involved in the anaerobic fermentation process rather than the C:N ratio available to them in pure straw mono-fermentation without recirculation of N-containing process water. At least in the case of anaerobic fermentation, the combined use of straw and farm manure is therefore advantageous. In a preferred further embodiment of the invention, straw and farm manure are therefore bacterially fermented anaerobically together and the fermentation residues are carbonized, preferably by pyrolysis and more preferably by high-temperature pyrolysis.

Since the method according to the invention and the system according to the invention produce GHG-negative energy carriers, they make it possible to add GHG-positive energy carriers, such as CNG or LNG. As shown above, the proportion of admixture is up to 90.8%. This means that up to 62,300 GWh_{Hi} CNG or LNG can be added to the GHG-negative gas fuel quantity of up to 68,600 GWh_{Hi} produced from farm manure and leaf waste without the use of straw, without the GHG emission value of the resulting mixed gas quantity of up to 130,900 GWh_{Hi} (471 PJ) turning positive. This additional quantity of mixed gas corresponds to another 20% of the calorific value of all fuels used in German road traffic in 2016.

In the case of co-fermentation of the entire German straw removal and the entire German volume of farm manure and leaf waste, the method according to the invention and the system according to the invention can thus provide up to 195,633 GWh_{Hi}+130,900 GWh_{Hi}=326,533 GWh_{Hi} (1,176 PJ) of absolutely GHG-free mixed gas annually, which corresponds to 50% of al fuels consumed by German road traffic in 2016.

If the current average energy efficiency in road transport is increased by 100% (the specific energy input is halved), the method according to the invention and the system according to the invention can provide up to 100% of the total amount of energy consumed by German road transport alone with the German straw growth and the national amount of farm manure and leaf waste and without further biomass imports, i.e. also the share of German fuel consumption accounted for by diesel propulsion (cars, light commercial vehicles and trucks as well as buses, tractors and special vehicles).

### 6. DETAILED DESCRIPTION OF THE INVENTION, FURTHER DEVELOPMENTS AND EMBODIMENTS

Preferred embodiments of the method according to the invention and preferred embodiments of the system according to the invention have been described and are described below by way of example. With regard to the additions to the teaching according to the invention, the inventor refers to the relevant prior art. It should be taken into account that both the basic concept of the invention and the embodiments can be modified and changed in many ways without leaving the basic concept and the basis of the invention. Patent protection is therefore also claimed for obvious modifications, alterations and additions to the invention.

The features of the invention disclosed in the description, the list of reference signs, the drawings and the claims can be essential for a beneficial development of the invention either individually or in any combination with one another.

The basic concept of the invention shall not be limited to the exact form or details of the embodiments shown and described below. Furthermore, it shall not be limited to a subject matter which would be limited in comparison to the subject matter described in the claims. In connection with specified design limits, values within the limits shall also be disclosed as possible values and shall be freely usable and claimable.

Relevant features, advantages and details of the invention result from the following detailed description, the list of reference signs, the drawings, the possible embodiments, the examples and the possible embodiments of the examples.

The invention is based on the finding that even in the best case scenario, the recuperation and sequestration of fossil carbon within the framework of fuel production only leads to a non-increase in the amount of greenhouse gases present in the atmosphere of the earth, i.e. the maximum GHG emission reduction performance is 100%. However, since the production, distribution and use of a fossil energy carrier or fuel usually involves the use of other fossil energy carriers, the fossil carbon of which cannot be recuperated, fossil energy products can usually only achieve high decarbonization effects or GHG emission reduction performances in theory.

The invention is based on the further finding that a partial recuperation and sequestration of atmospheric carbon within the framework of a fuel production process can lead not only to a GHG emission reduction performance of 100%, but also to GHG emission reduction performances that go far beyond this. If a fuel production process uses only those substances the carbon content of which consists of atmospheric carbon (which is the case, for example, with biomass, see below) and only a part of this atmospheric carbon is used to produce the fuel and the remaining carbon portion or a large part of this remaining carbon portion is recuperated and sequestered permanently in a carbon sink, a certain decarbonization effect results, which is usually associated with the product of the method. If the energy carriers required for biomass conversion are only loaded with low GHG emissions and thus play a relatively small role in the GHG balance or the GHG emission value of the energy carrier produced, the relationship between the carbon content entering the energy carrier and the carbon content permanently sequestered in a carbon sink is essentially decisive as to how high the GHG emission reduction performance of the sustainable energy carrier produced will be. If the relative share of carbon entering the sustainable energy carrier (biomass derived) is low and the relative share of sequestered (biomass derived) carbon is high, the resulting decarbonization effect related to the energy carrier produced is very high. Accordingly, for the energy carrier produced, which can be a fuel, there is a GHG emission reduction performance that can amount to several hundred percent or even more in relation to the energy unit produced (MJ or kWh_{Hi}) compared to the fossil reference.

Usually, the amount of sequestered carbon is multiplied by a factor of 3.664 to achieve the GHG effect. If carbon reacts with atmospheric oxygen to form CO₂, the molar fraction of carbon in the total molar mass of the CO₂ molecule is 12.0107 g/44.01 mg=27.291%; the mass of the CO₂ molecule is therefore 1/0.27291=3.664 greater than the mass of the carbon atom. Accordingly, the mass of avoided CO₂ emissions Is greater by a factor of 3.664 compared to the mass of sequestered atmospheric carbon.

The substances consisting of atmospheric carbon comprise all plants and plant-derived input materials, such as all animals and animal products, because plants have absorbed their carbon from the atmosphere of the earth by photosynthesis and animals are known to live from plants or other animals that eat plants. This means that the carbon in animals and animal products also consists of atmospheric carbon. Accordingly, the carbon content of biowaste also originates from the atmosphere of the earth.

The basic method according to the invention, for which protection is claimed, consists only of the three method steps 1.) Single-stage or multi-stage conversion of biomass containing atmospheric carbon into a marketable energy carrier, 2.) Generation of conditions allowing at least a partial chemical-physical stabilization of the atmospheric carbon still contained in the residues of the single-stage or multi-stage biomass conversion, 3.) Conduction of an at least partial chemical-physical stabilization of the atmospheric carbon still present in the biomass conversion residues (see claim 1). As long as the biochar/vegetable coal produced is protected from weathering and combustion, this stabilization of the atmospheric carbon is already sufficient to achieve the desired decarbonization effect because, except in the case of deliberate combustion, the carbon no longer reacts with atmospheric oxygen no matter where it is stored protected from the weather (which may be the case, for example, in mine tunnels and caverns).

The basic method can be advantageously supplemented by further method steps. In a preferred embodiment of the invention, the (originally first) method step of the single-stage or multi-stage conversion of the biomass into a marketable energy carrier is preceded by the additional method steps of selecting and/or harvesting or collecting at least one biogenic input material containing atmospheric carbon, preferably in that the selection is carried out on the basis of the following input material groups: cultivated biomass, straw (cereal straw, corn straw, rice straw, etc.; pure or as part of silage), straw-containing solid manure (solid cattle manure, solid pig manure, poultry manure, dry chicken manure, horse manure, etc.), straw-containing residues from mushroom cultivation, slurry, swill, fresh grass-like plants (ryegrass, switch grass, miscanthus, giant reed), catch crops before and after main crops, silages from grass-like plants, whole-plant corn cuttings, corn silage, whole-plant cereal cuttings, silage from whole-plant cereal cuttings, cereal grains, corn grains, wood, residues from biomass processing, by-product from the processing of biomass, cellulose-containing non-food material, waste paper, sugar cane bagasse, grape marc and wine lees, lignocellulose-containing biomass, residual forest wood, landscape conservation material, roadside greenery, cereals and other crops with a high starch content, sugar plants (sugar cane, sugar beets, industrial beets and the like), oil plants (palms, rapeseed, sunflowers, etc.), algae, biomass fraction of mixed municipal waste, household waste, biowaste, biowaste from private households, biowaste from industrial and/or commercial enterprises, biogenic waste from wholesale and retail trade, of the agricultural and food industry as well as the fishing industry and aqua industry, slaughterhouse waste, sewage sludge, waste water from palm oil mills, empty palm fruit bundles, tall oil pitch, crude glycerine, glycerine, bagasse, molasses, grape marc, wine lees, stillage from ethanol production, nut shells, husks, cored corn cobs, biomass fraction of waste and residues from forestry and forest-based industries (bark, twigs, pre-commercial thinnings, leaves, needles, tree tops, sawdust, black liquor, brewing liquor, fiber sludge's, lignin, tall oil), other cellulose-containing non-food material, other lignocellulose-containing material, bacteria, used cooking oil, animal fats, vegetable fats or combinations thereof. The GHG load or the GHG emission value of some of these input materials is particularly low in some cases. Since the GHG emission quantity and the GHG balance of a biomass-based fuel production path are essentially determined by the input material or the GHG emission value thereof, a corresponding selection of the input materials listed above is advantageous.

In another advantageous embodiment of the method according to the invention, at least a portion of the atmospheric carbon contained in the biomass is converted into a gaseous and/or liquid energy carrier (biogas, bio-methane, bio-ethanol, bio-diesel, FT-fuel, syn-diesel, bio-kerosene, syn-kerosene, bio-methanol, DME, butane, propane, etc.), so that a remaining portion of the atmospheric carbon passes into the method steps of stabilizing the carbon (generating the conditions, conducting the stabilization), preferably a portion of at least 0.1%, more preferably a portion of at least 40% and in particular a portion of at least 65%. Preferably, the proportion of the chemically and physically stabilized carbon in the original (at the start of the method) atmospheric carbon contained in the biomass reaches a selection of the following proportions: 0.1%, 5%, 10%, 15%, 20%, 25%, 30%, 35%, 40%, 45%, 50%, 55%, 60%, 65%, 70%, 75%, 80%, 85%, 90%, 95%, 100%, wherein each of the fractions given here may additionally vary within a range of at least +/−2.5% points, except for the fractions 0.1% where the range of variation may be −0.1% point to +2.4% points and 100% where the range of variation may be −2.5% point to 0.0% points.

In a preferred embodiment of the invention, the at least partial chemical-physical stabilization of the atmospheric carbon still contained in the residues of the biomass conversion is carried out by a chemical-physical treatment of the conversion residues, preferably by carbonization of the conversion residues to biochar/vegetable coal/biocoke, more preferably by a selection from the following carbonization methods: pyrolysis, carbonization, torrefaction, hydrothermal carbonization (HTC), vapothermal carbonization, gasification and any combination of these treatment methods.

In an advantageous embodiment, the biochar/vegetable coal/biocoke produced according to this basic method and with it the at least partially chemically and physically stabilized atmospheric carbon are at least partially sequestered, in an additional method step, in the ground, in stagnant waters, in aquifers or in the ocean, preferably in agricultural or forestry soils, more preferably in soils that are not or no longer used in agriculture or forestry, and in particular in desert or permafrost soils and, at best, in another carbon sink. The sequestration of the biochar/vegetable coal/biokoke or the at least partially chemically and physically stabilized atmospheric carbon can therefore also include its final storage in geological formations, aquifers or other waters. It is preferred that the sequestered carbon content of the atmospheric carbon originally contained in the biomass (at the start of the method) reaches a selection of the following proportions: 0.1%, 5%, 10%, 15%, 20%, 25%, 30%, 35%, 40%, 45%, 50%, 55%, 60%, 65%, 70%, 75%, 80%, 85%, 90%, 95%, 100%, each of the proportions given here additionally varying within a range of at least +/−2.5% points, except for the fraction 0.1% where the range of variation may be −0.1% point to +2.4% points and the fraction 100% where the range of variation may be −2.5% point to 0.0% points.

Preference is given to a method variant in which the energy carrier produced is processed in the method step of the single-stage or multi-stage conversion of biomass into an energy carrier in such a way that it can be used as fuel, heating medium or combustion material, preferably as traffic fuel, more preferably as road fuel.

Preferably, the energy carrier used as fuel, heating medium or combustion material consists of biogas, bio-diesel, bio-ethanol, bio-kerosene, hydrogen, bio-methane, FT-fuel, DME, butane, propane or bio-methanol.

Due to a weather and soil organism-resistant storage of the atmospheric carbon, which can but does not have to be sequestration, the technical GHG balance or the GHG emission quantity of the produced energy carrier becomes strongly negative, i.e. after completion of the production, distribution and use of the produced energy carrier, which is preferably a fuel, more preferably a gas fuel and in particular bio-methane, there is a lower greenhouse gas quantity in the atmosphere of the earth than before. The high GHG negativity (or negative GHG emission quantity) of the produced energy carrier allows the addition of such a quantity of a suitable (compatible) positively GHG-loaded energy carrier that an absolutely GHG-free energy carrier is produced (suitable or compatible here means “same fuel type and same aggregate state”). This means that bio-ethanol and/or lingo-ethanol produced in this way can be mixed with fossil gasoline, bio-diesel produced in this way with fossil diesel, bio-kerosene produced in this way with fossil kerosene, bio-methane produced in this way with fossil natural gas (CNG or LNG) and/or syn-methane, FT-diesel produced in this way with fossil diesel, syn-kerosene produced in this way with fossil kerosene, hydrogen produced in this way with hydrogen formed from natural gas by steam, etc. Therefore, a quantity of an appropriate compatible (suitable) GHG-positive energy carrier can preferably be added to the energy carrier produced according to the method of the invention in such a way that the GHG emission value of the energy carrier mixture does not just yet change into positive or remains negative.

The GHG-free energy carrier mixture makes all vehicles which refuel the GHG-neutral energy carrier mixture, independent of their size and consumption, real zero-emission vehicles. If the admixture of GHG-positive energy carriers is low or completely absent, the method according to the invention produces a strongly GHG-negative energy carrier, the use of which is very positive for the environment, because after completion of the production, distribution and use of the produced energy carrier, which is preferably a fuel, more preferably a gas fuel and in particular bio-methane, there is a smaller amount of greenhouse gas in the atmosphere of the earth than before.

The invention preferably consists of an extended method and extended plants for straw fermentation and suitable devices, which make it possible to convert straw into GHG-negative biogas, to process the GHG-negative biogas into GHG-negative (bio-)methane, to mix it with (GHG-positive) natural gas to form a GHG-neutral mixed gas, to feed the mixed gas into the natural gas grid and to transfer the energy equivalent of the injected quantity of mixed gas at any exit points to gas filling stations which deliver it to CNG and LNG vehicles which, irrespective of their size and fuel efficiency due to the GHG-neutral fuel, are immediately on the road as zero-emission vehicles without any GHG emissions. Alternatively, GHG-negative (bio)-methane can also be mixed with fossil natural gas in the natural gas grid or, if both components are liquefied, in an LNG tank.

In a further embodiment, GHG emissions not yet taken into account can also be taken considered for the formation of the mixed gas, preferably GHG emissions proceeding from the use of electricity, more preferably GHG emissions proceeding from the use of fuel and heating mediums and in particular those GHG emissions which only occur downstream after the mixing, such as GHG emissions resulting from the compression of the mixed gas withdrawn from the natural gas grid to the discharge pressure (usually 250-300 bar) and/or as methane slip during the refueling of vehicles and/or the liquefaction of the mixed gas to the LNG substitute “liquefied bio-methane” (LBM). This means that a slightly GHG-negative mixed gas is produced, the absolute GHG emission reduction quantity of which is just as large as the absolute GHG emission quantity associated with the downstream effects.

A more and more frequently demanded turning away from the proven drive technology “Internal combustion engine” is no longer necessary when using the technology revealed here. It is only necessary to switch from the gasoline combustion technology to the methane combustion technology, which essentially corresponds to natural gas combustion. This has proved its worth, as demonstrated not least by the about 1.9 million CNG vehicles (of which 1.427 million passenger cars and light commercial vehicles, 0.275 million buses and 0.195 million heavy commercial vehicles) already in use throughout Europe. A positive aspect is that the GHG-free fuel “mixed gas” can also be provided in liquid form as an LNG substitute by simple liquefaction, which is of particular interest for heavy goods vehicles.

Not least because of the relatively large production volume, the new technology disclosed here can prevent the more and more frequently demanded abolition of the internal combustion engine, the production of which shall be discontinued as early as 2030 due to the long service life of a car, so that zero-emission mobility can be achieved in 2050. With the absolutely GHG-free zero-emission fuels of the method according to the invention and the system according to the invention, a comprehensive zero-emission mobility is already possible today despite the use of combustion technology, which from the point of view of customers and users is clearly superior to both electric mobility and H₂ mobility.

For the customer, everything remains the same: the refueling still only takes a few minutes, the range of a tank filling is several hundred kilometers (with the fuels natural gas and gasoline there are even 2 back-up options), the life span of the tank is not limited (the problem of the maximum charge cycles does not exist), in winter the heating can be used without restriction and in summer the air conditioning system without the range decreasing, the acquisition costs and the depreciation of the vehicle are smaller than with the electric car and also still smaller than with the diesel car of the European norm 6, the motor vehicle tax is very much smaller than with diesel cars. Above all, there are no restrictions on use in terms of payload (which is important for commercial vehicles) and speed (which is important for most German car drivers). With regard to emissions there is also a very advantageous technical quantum leap: real driving emissions (RDE) are reduced by 100% compared to gasoline and diesel cars in terms of greenhouse gases, by 85%-90% in terms of NOₓ emissions, by 99% in terms of particulate matter emissions and by 67%-76% in terms of emissions of toxic hydrocarbon compounds. The LCA emission values of the gas vehicles powered by the mixed gas according to the Invention (CNG and LNG) are thus considerably better than those of all other propulsion systems, including electric and hydrogen vehicles (electric cars use the national or European electricity mix, both of which are loaded with emissions from coal-fired power generation and/or the risks of nuclear power well after 2040; the GHG load of the German electricity mix (domestic consumption) amounted to 587 gCO₂-eq/kWhₑₗ in 2015 according to the Federal Environment Agency; the higher net drive efficiency of the electric car can only partially compensate for this high GHG value, so that electric cars are currently only about as environmentally friendly as gas vehicles without having their advantages; the hydrogen used by hydrogen cars is largely produced by steam reforming from natural gas, which makes it even dirtier than the original input material, i.e. natural gas, due to the energy conversion losses).

The method according to the Invention thus delvers “affordable” zero-emission mobility despite the use of the Internal combustion engine. This method thus secures jobs in the automotive industry and its suppliers because established Industrial plants (facilities for the production of crankshafts, connecting rods, cylinder heads, camshafts, etc., engine plants and gear factories) can continue to be manufactured and used.

In contrast to competing processes for producing lignocellulose-ethanol from straw and for producing Fischer-Tropsch fuel from straw, the method according to the invention is not only much simpler but also much more efficient in terms of converting the biomass used. The conversion efficiency in the production of lignocellulose-ethanol from straw is about 40%, in the production of Fischer-Tropsch fuel from straw 29%-37%, in the production of GHG-negative straw-gas from straw about 70% and in the production of GHG-neutral mixed gas about 125%. This means that while methods for producing ligno-ethanol extract about 1,600 kWh_{Hi} of (GHG emission-reduced) fuel from one ton of straw (wet mass), FT methods achieve only 1,200-1,500 kWh_{Hi} of (GHG emission-reduced) fuel and the method according to the invention 2,860 kWh_{Hi} of pure (strongly GHG negative) straw-gas or 5,100 kWh_{Hi} of (GHG neutral) mixed fuel. In addition, the particularly high GHG emission reduction performance, the increase of which to well over 100% is equivalent to a quantum leap, means that the number of vehicles that can be supplied with zero-emission fuel can be increased many times over (see above).

Despite these outstanding performances, the technical and economic effort required by the method according to the invention and the system according to the invention is much less demanding than in the production of lignocellulose-ethanol from straw and also much less demanding than in the production of FT-fuels from straw. The (special) biogas plants used do not only have a considerably higher conversion efficiency (70% compared to 29% to 40%), they also remain at a medium level, so that the plants specialized in the fermentation and pyrolysis of straw do not require large industrial catchment areas and can therefore be set up and operated in a decentralized way.

The invention described here is therefore much better suited for practical use than the less efficient and more costly competitive processes. Not least for this reason, the inventor and the applicants are of the opinion that his development has breakthrough potential and that the innovative system makes the often propagated new invention of the automobile superfluous.

Procurement of Straw

In the production, distribution and use of energy carriers from biomass, more or less high GHG emissions are still produced without stabilization and/or permanent sequestration of atmospheric carbon, depending on the type of biomass. According to the publications of the German Federal Institute for Agriculture and Food (FAF), the GHG emission reduction performance of both the biofuel, i.e. bio-ethanol, and the biofuel, i.e. bio-diesel, compared to the fossil reference was on the average about 70% in 2015, the residual emission thus still being about 30% of the fossil reference. It is therefore advantageous to select input materials, the GHG footprint of which is as small as possible, especially if the GHG emission reduction performance of the fuels, heating mediums and combustion materials produced shall be as high as possible. According to the invention, input materials are thus selected which have little or no GHG emissions (cf. claim 21). Among the input materials that are not initially polluted with GHG emissions is straw in particular, which according to EU Directive 2009/28 (RED 1) is not loaded with GHG emissions until the time of collection/harvest. The GHG emissions resulting when the cereal plants are cultivated and harvested are allocated solely to the cereal grains. It goes without saying that all other biogenic substances, such as wood, can also be used; the GHG balance or the GHG emission value is only then not quite as good as when straw is used. It is therefore advantageous if straw or straw-containing conversion residues are used in the method according to the invention.

As a by-product of the cereal grain, straw is only produced during a short period of time, i.e. during the cereal grain harvest in summer and early autumn. Since industrial, straw-using biogas plants are in operation all year round (up to 8,760 hours a year) and require fresh input materials daily, it is necessary to store large amounts of straw or store it temporarily. For larger quantities, this is usually decentralized, which results in multi-stage logistics processes.

The supply chain begins when the mown and threshed straw is deposited in the swath behind the self-propelled combine harvester, which switches off its chopping system for this purpose. The loose straw deposited in the swath has a density of about 25 kg/m³ and is therefore not suitable for transport. Compaction is necessary to achieve transport suitability. For this purpose, a baler pulled by an agricultural tractor picks up the straw swath and compacts the native straw into straw bales. The straws can keep their length from 20 cm to 120 cm when compacted into straw bales or can be chopped into straw chaff, which can be 5 cm to 20 cm long. The straw bales can be round bales or square bales. If larger transport distances are part of the logistics chain, square bales are preferred, especially square bales made with high pressure balers. While round bales have a density of 110-130 kg/m³, conventional square bales have a density of 130-165 kg/m³ and high pressure bales 170-210 kg/m³. The transport suitability of the straw increases with increasing density. In the case of very long transport distances, pellet presses pulled and driven by agricultural tractors (so-called pellet harvesters) can also pellet the straw swath directly into straw pellets, which increases the density to up to 600 kg/m³ and further increases the transport suitability.

Depending on the design of the straw baler, rectangular or round bales or straw pellets with different sizes and densities are produced. The pressed bales are preferably placed in groups on the stubble field. This facilitates the subsequent harvesting steps of collecting and loading the first means of transport.

A modern square baler usually has a throughput of 35 t fresh straw mass per hour. The tractor should have an output of at least 150 kW. Such tractors consume about 18 liters of diesel equivalent per hour of operation, i.e. a (heating) energy quantity of about 178 kWh_{Hi}/h. When conventional mineral diesel is used, this fuel input is associated with a greenhouse gas emission of 178×342.36=60,940 gCO₂-eq (according to EU Directive EU 2015/652 of 20 Apr. 2015, the weighted life cycle greenhouse gas intensity for diesel fuel is 95.1 gCO₂-eq/MJ, which corresponds to 342.36 gCO₂-eq/kWh_{Hi}). Each ton of straw thus accounts for an energy input of about 5 kWh_{Hi} and GHG emissions of 1,741 gCO₂.

The method according to the invention involves the use of tractors with CNG or LNG engines that use GHG-free straw-gas or a GHG-neutral fuel mixture as fuel. The first tractors with CNG drive already exist. They can be used just like conventional tractors. The energy required to press the straw remains almost the same when using CNG or LNG tractors which refuel with a GHG-neutral gas fuel. They thus also amount to about 5 kWh_{Hi}/t straw-WM, only the GHG emission goes back to 0.0 gCO₂/kWh_{Hi} and thus also to 0.0 gCO₂-eq/t straw-WM.

Agricultural companies usually use existing technology to collect the bales of straw and load the first means of transport. Front loaders or so-called manitous pick up the bales individually or in pairs and load agricultural means of transport for the first transport to the straw haystacks. This practice is relatively time and energy intensive.

If larger quantities of straw are to be pressed into bales, new technology is used. Wheel loaders with 6-fold multiple grab tongs, e.g. from the Dutch company Meier, can load up to 6 square bales at a time onto trucks with semi-trailers within a very short time. Collecting and loading 6 large square bales onto a semi-trailer takes just 180 seconds, i.e. 30 seconds per bale. A square bale with the usual dimensions of 1.20m×0.90m×2.40m=2.592 m³ usually has a density of 160 kg/m³, so that the straw wet mass per bale is 415 kg. With a normal fuel consumption of 17 liters of diesel equivalent per operating hour, 0.142 liters of diesel equivalent or 1.4 kWh_{Hi} are required per bale to collect and load. The use of diesel results in GHG emissions of 342.36 gCO₂-eq/kWh_{Hi}×0.142 kWh_{Hi}=48.6 gCO₂. Based on one ton of straw, the energy consumption is 1.4/0.415=3.37 kWh_{Hi} and the GHG emission is 48.6/0.415=117.1 gCO₂/t straw-WM. When wheel loaders with CNG or LNG drive are used according to the invention and the new GHG-neutral gas fuel mixture is used according to the invention, the energy consumption for collecting and loading the straw bales remains at 3.4 kWh_{Hi}/t straw-WM, because the CNG/LNG drive technology is almost as efficient as conventional drive technology. Only the GHG emission drops to 0.0 gCO₂/t straw-WM.

With conventional loading, tractors with front loaders load low-floor loader wagons towed by tractors or low-floor semi-trailers with double-axle mountings. They transport the bales over relatively short distances (up to 10 km) to a straw store, where the up to 3.5 m³ large and up to 0.7 t heavy straw bales are unloaded from the loader wagons with telescopic loaders and stacked to so-called straw haystacks. In the method according to the invention, articulated trucks with low-floor trailers collect the straw bales directly from the field. Wheel loaders collect them with multiple grabs and load them onto the truck as 6-pocks. Loading takes about 30 seconds per bale (see above).

Depending on the bale dimensions, the truck has a loading capacity of 3-4 layers of 11-12 bales each, so that loading the total of 36-48 bales only takes about 18-24 minutes. With a straw bale dimension of 1.20m×1.00m×2.40m=2.88 m³ and a density of 0.165 t/m³, the bale weight is 475 kg. The loading with 3 layers of 12 bales each results in a loading weight of 17.1 tons, which approximately utilizes the loading capacity of the truck. Increasing the bale density to 0.180 t/m³ and changing the bale size to 1.20m×0.90m×2.40m=2.59 m³ results in a bale weight of 467 kg and, with 4 layers of 11 bales each, a loading weight of 20.5 t, whereby the loading capacity of the truck is fully utilized. By optimizing the bale shape and density, it is therefore possible to maximize the loading capacity of the trucks not only in terms of loading volume but also in terms of weight.

In order to save the steps of unloading, building a decentralized straw haystack, removing this straw haystack and reloading a truck in the logistics chain, the straw is transported according to the invention directly from the field to a central storage location near the biogas plant. The truck trains transport the straw bales via highways to the biogas plants, where they are unloaded either by telescopic loaders or by cranes equipped with multiple grabs. If the biogas plant is located on a shipping lane or near a port, the straw can also be delivered and discharged in the form of pellets.

With an average procurement distance (distance from the field or from the decentralized straw storage to the biogas plant) of 50 km and a load of 20 t of straw, the lorry provides a transport capacity of 1,000 tkm per load. With a consumption of 33 liters of diesel equivalent per 100 km, an energy quantity of about 163 kWh_{Hi} is used for long-distance transport, and with a consumption of 28 liters for the empty journey back to the decentralized warehouse a further 137 kWh_{Hi} is used. In total, the energy expenditure for long-distance transport therefore amounts to about 300 kWh_{Hi} per load. Based on the transported calorific value of 20×4,085 kWh_{Hi}=81,700 kWh_{Hi}, this is just 0.37% (15 kWh_{Hi}/t straw-WM). The transport effort is therefore very low even if the average transport distance is increased to the exceptionally long distance of 250 km.

If conventional diesel fuel were used, 15 kWh_{Hi}×342.36 gCO₂-eq/kWh_{Hi}=5,134 gCO₂-eq would be emitted into the atmosphere per ton of straw-WM. However, since the method according to the invention provides that the trucks will be equipped with CNG or LNG engines that refuel and use GHG-free mixed gas produced according to the method of the invention, the long-distance transport of the straw will not cause any GHG emissions.

In the biogas plant, the straw bales are handled in the same way as in large combined heat and power plants using straw and a gantry crane with multiple grabs and conveyor belts. It is also possible to supply bulk goods with straw pellets, which are then stored in suitable silos.

Storage in the central warehouse will mainly take place in the form of bales. As cranes with multiple grabs are used and are operated electrically and thus with high efficiency, the energy consumption and GHG emissions for unloading the trucks and setting up the central straw haystacks are negligible. The removal of the straw bales from the central warehouse, the transport to the biogas plant and the handling of the straw bales in the biogas plant is carried out with stationary conveyor technology, which is also electrically operated and therefore highly efficient.

Proven experts of the German Biomass Research Center GBRC calculate for a harvest quantity of 40.000 t straw-WM per year for pressing, collecting and loading, first transport with agricultural subsidies, unloading and stacking to a (first) decentralized straw haystack, removal of the bales from this first straw haystack, loading of the truck trains and unloading of the truck trains at the biogas plant without transport to the biogas plant—a total energy consumption of 33.5 kWh_{Hi}/t straw-WM. The method according to the invention reduces this energy consumption per ton of straw-WM to 5.0 kWh_{Hi} for pressing, 3.4 kWh_{Hi} for loading the truck and 15 kWh_{Hi} for long-distance transport, i.e. a total of 23.4 kwh_{Hi}.

If pure mineral diesel fuel were used without the addition of biofuels, this energy consumption would cause a GHG emission of 23.4×342.36=8,011 gCO₂-eq. However, since the method according to the invention provides CNG drives or LNG drives for both the tractors used and the wheel loaders and trucks that run on GHG-free gas fuel, the GHG emission caused by collection, loading and transport is 0.0 gCO₂-eq/t straw-WM.

Conversion of Straw

According to the method of the invention, any known biomass conversion can be used for the first single-stage or multi-stage biomass conversion, the aim or task of which is to convert the biomass into a (marketable) energy carrier. However, preference is given to an embodiment and a corresponding system for carrying out this method, in which at least part of the biomass to be converted consists of straw.

Preferably, the first biomass conversion is a conversion of straw-containing biomass into energy carriers, more preferably a selection of the following conversion processes: Conversion of straw-containing biomass into bio-diesel, conversion of straw-containing biomass into bio-ethanol, conversion of straw-containing biomass into ligno-ethanol, conversion of straw-containing biomass into Fischer-Tropsch fuels, conversion of straw-containing biomass into methanol, conversion of straw-containing biomass into DME, conversion of straw-containing biomass into hydrogen, conversion of straw-containing biomass into biogas and combination of these conversion processes.

In an advantageous embodiment, the method according to the invention and the system according to the invention produce the GHG-negative gas, i.e. bio-methane, from straw, which can be distributed in gaseous form as a natural gas substitute or in liquefied form as an LNG substitute. The liquefaction of (bio-)methane to liquefied (bio-)methane (LBM) is just as much a known prior art as the processing of biogas to bio-methane.

In an advantageous embodiment of the method and system according to the invention, the first biomass conversion consists of a conversion of straw-containing biomass into biogas, more preferably a conversion of straw-containing biomass into biogas, which is carried out according to the solid fermentation process and in particular from a solid fermentation, in which garage fermenters, plug flow fermenters or up-flow fermenters are used. In particular, the at least one garage fermenter is operated with a fermentation cycle shorter than 24 days, in particular shorter than 15 days and at best shorter than 9 days.

In an alternative advantageous embodiment of the method and system according to the invention, the first conversion takes place as anaerobic bacterial fermentation according to the wet process. Prior to conversion of the biomass, which preferably consists at least in part of straw and/or straw-containing input materials, into a marketable energy carrier, a suspension is produced from the biomass and a liquid, preferably from the biomass and an aqueous suspension, more preferably from the biomass and process water. The suspension here has a dry substance content (DS content) of 1%-60%, preferably a DS content of 5%-30%, more preferably a DS content of 8%-18% and in particular a DS content of 9%-14%.

Straw is not an input material like any other biomass; it has specific properties that make it unusually difficult to process or use. Native straw—i.e. straw that is not pre-processed and is in its natural state—is a very difficult material to use because of its specific properties [in particular: particle length of straws 20-120 cm; waxy surface; fibril structure; microfibril structure; high fiber content; high lignin content; high strength; very wide C/N ratio of 70-100; high potassium content; high chlorine content; very low density of about 25 kg/m³; very high DS content and correspondingly very low residual water content; difficult to comminute; increased tendency to dust formation and associated increased risk of explosion; high surface tension; low solubility in water; when burnt, significant differences to wood combustion, such as low softening temperature, tendency to tar formation and sintering of the combustion chamber, higher ash content by a factor of 10, much higher chlorine and nitrogen content in the flue gas, significantly higher dust emissions etc.] no input material like any other solid, in particular not when used in pelleting facilities, mills, anaerobically bacterial biogas facilities, anaerobically enzymatic fermentation facilities, aerobically composting systems, firing facilities and waste treatment facilities. If these facilities process other solid input materials, they are still for from being able to process native (long) straw.

For example, the industrial pelleting of straw requires special pelletizing systems designed for the “straw” input material. The same applies to heating or combustion devices that utilize straw. The input material “straw” also requires special grinding technology for grinding. For example, mills that grind small grain sizes into flour are only suitable for grinding long straw if the straw has previously been comminuted. Native (long) straw consists of straws with a high fiber content, which usually have a length of 20-70 cm and can sometimes be up to 120 cm long. In practice, native straw cannot be used in standard mills. Fibrous materials, such as straw, therefore require a special milling technique. It is thus a false conclusion to assume that any solid biomass processing facility would also be suitable for processing native (long) straw. In order to be able to use (long) straw with standard technology in practice, as a rule at least chopping devices are required. In practice, for example, upstream comminution of the native long straw can only be omitted if a special device designed for straw utilization is used.

As the patent EP 2167631 of the inventor shows, the lignin content, which is indigestible for microorganisms and accounts for about 21% of the dry straw mass, blocks the path to cellulose and hemicellulose, which are partly converted into biogas, during straw fermentation. In particular, native, undigested straw can only be converted to a small extent into biogas by biogas facilities within the usual retention times (Hydraulic Retention Time HRT) of 20-60 days.

In addition, the relatively low nitrogen content in the straw results in a very wide C/N ratio of the straw. This ratio is typically 70-100. Anaerobic microorganisms require a C/N ratio of 6-20 for their growth and propagation. Therefore, the fermentation of straw requires either recuperation plus recirculation of nitrogen-containing suspensions into the process or an addition of one or more nitrogen-containing fermentation substrates, such as poultry manure, which has a particularly narrow C/N ratio.

The use of straw as an anaerobic fermentation substrate therefore requires special pre- and/or post-treatment measures, including the digestion of native straw and/or the addition of nitrogen/nitrogen compounds or fermentation substrates with a very low C/N ratio. The relevant prior art also discloses the digestion and pre/post-treatment measures of chopping, grinding, soaking, mashing and the pre-treatment with hot water, steam, saturated steam, thermal pressure hydrolysis, wet oxidation, steam reforming, steam explosion, etc., as well as the support of the first phase of anaerobic fermentation (hydrolysis) by the application of exo-enzymes. Also previously known are the recuperation and recirculation of (If necessary processed or purified) process water into the process, the recuperation and recirculation of nitrogen-containing suspensions into the process, the pre-heating of fermentation substrates prior to fermentation for removing the wax layer on the straws, the repeated fermentation of fermentation residues from a first fermentation after treatment with hot water, steam, saturated steam, thermal pressure hydrolysis, wet oxidation, steam reforming, steam explosion, etc., the recuperation and recirculation of process heat (inter ala heat exchange in the countercurrent process), the biological pre-treatment of fermentation substrates with fungi, the biological post-treatment of fermentation residues with fungi, the removal of pollutants from process waters (e.g. by filtration, ultrafiltration, reverse osmosis) and the spreading of straw-containing fermentation residues on agricultural and other areas to maintain their humus content. It should be understood that all previously known and obvious pre-treatment and digestion measures and all known measures for post-treatment and use of the fermentation residues can be combined with the method steps listed and explained below. A person skilled in the relevant art knows from the relevant prior art how this has to be done.

In the case of anaerobic fermentation substrate, i.e. straw, the achievable conversion efficiency and the dynamics of biogas production (consideration of the time factor) depend decisively on the type of pre-treatment. If the pre-treatment consists of several days of aerobic composting, the biogas yield decreases significantly, as in particular the easily accessible and easily digestible carbon compounds are oxidized to form CO₂. The latter, in turn, diffuses from the reaction mass into the atmosphere and is then no longer present, which means that part of the carbon is lost.

As current practical values show, a conversion efficiency of up to 75% can be achieved with a pre-treatment combination consisting of straw grinding and saturated steam treatment and with a subsequent agitated wet fermentation of the straw digested in this way at an optimum C/N ratio of 30 maximum and without aerobic pre-rotting. The pretreatment “straw grinding only” results in a conversion efficiency of about 50% for agitated wet fermentation with an optimum C/N ratio of about 30 without aerobic pre-rotting and without steam treatment (saturated steam, TH, steam reforming, steam explosion and the like).

The fermentation of native straws, which were not ground but were also subjected to aerobic pre-rotting (composting) before fermentation and which are fermented with an unsuitable C/N ratio of 70-100 in an unagitated solid fermenter, achieves—if at all—only a maximum conversion efficiency of 20%.

In an advantageous embodiment of the invention, the biomass to be converted into a GHG-emission-reduced marketable energy carrier (fuel, heating medium or combustion material) is subjected to a conversion, which can preferably be an anaerobic fermentation or an alcohol fermentation, only after a suitable upstream measure previously known from the relevant art, preferably from the following measures: Mixing with water, aqueous suspensions or process water, soaking with water, aqueous suspensions or process water, comminution (bale disintegration, chopping/shredding, grinding, etc.).) of the biomass residues from the first fermentation, extrusion of this biomass, treatment of this biomass without pressure and with hot water or steam, pressurized treatment of this biomass with hot water or steam, treatment of this biomass with saturated steam, thermal pressure hydrolysis of this biomass, wet oxidation of this biomass, steam explosion of this biomass, steam reforming of this biomass, other known pre-treatment of this biomass, any combination of these measures.

In another advantageous embodiment of the invention, the biomass to be converted into a GHG-emission-reduced energy carrier is subjected to a multiple (two to ten times), preferably a double conversion, which is more preferably a double anaerobic fermentation, a double alcohol fermentation or a combination of anaerobic fermentation and alcohol fermentation; preferably, the second conversion takes place after an intermediate measure, which consists of a suitable measure previously known from the relevant prior art and which is more preferably selected from the following measures: Mixing with water or aqueous suspensions, comminution (dissolution, chopping/shredding, grinding) of the conversion residues from the first conversion, extrusion of these conversion residues, treatment of these conversion residues without pressure and with hot water or steam, pressurized treatment of these conversion residues with hot water or steam, treatment of these conversion residues with saturated steam, thermal pressure hydrolysis of these conversion residues, wet oxidation of these conversion residues, steam explosion of these conversion residues, steam reforming of these conversion residues, other known after-treatment of conversion residues which shall be subjected to further conversion, any combination of these measures.

In another advantageous embodiment of the invention, the biomass is subjected to a thermal treatment before conversion, preferably a multi-stage first tempering and second tempering, more preferably a multi-stage first, second and third tempering and in particular a multi-stage first, second, third and fourth tempering. The temperature control of the biomass can be carried out at temperature levels consisting of a selection of the following temperatures under the secondary condition that the subsequent temperature level Is higher than the previous one:

0.1° C., 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C. 155° C. 160° C. 165° C. 170° C., 175° C., 180° C. 185° C. 190° C., 195° C., 200° C., 205° C., 210° C., 215° C., 220° C., 225° C., 230° C., 235° C., 240° C., 245° C., 250° C., 255° C., 260° C., or any combination thereof, wherein each temperature value herein may additionally vary within a range of at least +/−2.5° C. The purpose of the multi-stage temperature control Is an advantageous softening or elimination of such structures and substances in the biomass which prevent or hinder the subsequent conversion.

In a further advantageous embodiment, before, during or after the step of the single-stage or multi-stage conversion of the biomass, at least a portion of the biomass Is replaced or supplemented by suitable input materials previously known from the relevant prior art, preferably by input materials from the selection of percolate, slurry, manure, grass, hay, grass silage, corn silage, cereal whole plant silage, silage from straw and another silage substrate, hay, cereal grains, potatoes, industrial beets, sugar beet, sugar cane, molasses, field beans, wildflowers, landscape maintenance, roadside greenery, residues from processing agricultural products, stillage from ethanol production, rapeseed, rapeseed press cake, cultivation biomass, wood, wood waste, biowaste, biogarbage, organic residues, residues from biomass processing, by-products from biomass processing, cellulose-containing non-food material, lignocellulose-containing input material, forest residual wood, cereals and other crops with high starch content, sugar plants, oil plants, algae, biomass fraction of mixed municipal waste, household waste, straw, straw-containing input materials, sewage sludge, waste water from palm oil mills, empty palm fruit bundles, tall oil pitch, crude glycerin, glycerin, bagasse, molasses, grape marc, wine trub, nut shells, husks, cored corn cobs, biomass content of waste and residual materials from forestry and/or forest-based industries (bark, branches, pre-commercial thinning wood, leaves, needles, tree tops, sawdust, sawdust, black liquor, brown liquor, fiber sludge, lignin, tall oil), other cellulose-containing non-food material, other lignocellulose-containing material, bacteria, used cooking oil, animal fats, vegetable fats, solid manure, dry chicken feces, poultry manure, straw-containing mushroom residues and any combination thereof.

In an advantageous embodiment of the invention, the fermentation of the straw does not take place in a classical “Wet plant”, which works according to the stirring tank principle with a slurry-like aqueous suspension, but in a solid fermentation plant, which works according to the garage principle. Preferably, the at least one garage fermenter Is operated with a fermentation cycle which lasts shorter than 24 days, in particular shorter than 15 days and at best shorter than 9 days. The anaerobic fermentation of the straw shall not be limited to solid fermentation plants by this example, it can also include the wet process, in principle, which is carried out in classical wet-type plants.

If the straw bales are not subjected to saturated steam treatment as a whole before fermentation (see patent application EP15001025.4 of the inventor), they are broken up before fermentation. The loose straw is then comminuted with a shredder and a grinding device. The degree of comminution can be varied so that the straw particles have a length of between 0.01 mm and 30.0 mm on the average. The comminution can therefore only include the chopping of the straw (to about 5 cm-20 cm) but also an additional grinding, e.g. with hammer mills or cutting mills. The degree of comminution depends on the conversion efficiency to be achieved: the higher the degree of comminution, the higher ceteris paribus the conversion efficiency. A high degree of comminution requires a high energy input (for the mills).

The bacterial fermentation of the straw Is preferred to ensure a C/N ratio of 20-40 together with nitrogen-containing fermentation substrate (poultry manure, slurry) and/or together with nitrogen-containing process water, which Is more preferably extracted from the fermentation residues beforehand. In anaerobic bacterial fermentation, only very few N components of the fermentation substrate introduced into the fermenter are converted into biogas; the for greater part remains in the fermentation mass, resulting in N enrichment in the fermentation mass or in the fermentation residues. If the fermentation substrates are enriched with N-containing process water before or during fermentation, the input quantity of N-containing fermentation substrates can be reduced. This reduction is advantageous for the properties of the biochars/vegetable coals/biocokes produced from fermentation residues.

The solid fermentation preferably takes place in a solid dust-like heap, which is showered (percolated) with percolate from above after closing the “garage door” under exclusion of air in the “garage”. In this process, the heap-like fermentation mass Is sucked up to the limit, at which free (process) water or a free suspension (percolate) is formed, with this percolate which consists of the seepage juice of the fermentation mass of the fermentation cycle previously completed and/or of process water produced in subsequent process steps. The bacteria-containing percolate seeps through the fermentation mass heap, is recuperated and is available for another showering. The anaerobic bacterial fermentation, which takes place as in a wet-type plant, can only be carried out in one stage in the garage fermenter or in two stages. In the two-stage process, at least part of the circulating percolate loaded with organic acids is fed into a high-performance methanizer in which immobilized microorganisms carry out the known methanogenesis. The percolate, which is lightened by a part of the organic acids, is discharged from the methanizer and percolated onto the fermentation mass heap and the circulation starts again.

In the so-caged garage process, the fermentation takes place in cycles that usually last 21-28 days. The introduction of the fresh fermentation mass into a plurality of garage fermenters is usually carried out with a wheel loader, as is the prior mixing of the fresh fermentation mass with inoculant (part of the old fermentation mass from the previous fermentation cycle) and the removal of the fermented fermentation mass from the garage fermenters. The garage fermenters are operated in time-staggered fashion so that biogas production is more or less continuous altogether.

On the one hand, the garage process has the advantage that no liquid fermentation mass is used, in which pieces of straw usually float up, form floating layers and block overflows. The straws are trapped in the solid manure-Ike heap. On the other hand, the straw-containing fermentation residues are not present in small, liquid form, but as a heap with DS contents of about 30%-40%. While liquid fermentation residues can only be subjected to one HTC without an unusually high technical effort (see above), the solid manure-like consistency of the fermentation residues makes the desired pyrolysis possible, preferably after any necessary dewatering of the fermentation residues to about 50% DS. Which process steps are used to convert straw into biogas depends on the conversion efficiency to be achieved. High conversion efficiencies require more technical and energetic effort than low ones. Both in the garage process and in the wet fermentation process, the pre-treatment of the fermentation substrates and/or the post-treatment of the fermentation residues are of great importance. In practice, the pre-treatment and post-treatment has been proven to be comminution, in particular grinding, which is preferably carried out after shredding, more preferably after bale breaking, followed by shredding and grinding. Other pre-treatment and post-treatment measures include mixing or treatment with warm water (15° C.-99° C.), mixing or treatment with hot water (>99° C.), gradual heating of the fermentation mass, treatment with (saturated) steam, with thermal pressure hydrolysis, with wet oxidation, with steam explosion, with steam reforming, biological treatment with fungi, blending or mixing with process water (If necessary treated or purified), re-fermentation after post-treatment and the combination of these measures have proved successful.

Irrespective of the desired conversion efficiency, with a fermenter size of 7 m wide×5 m high×30 m long about 60 t straw-WM can be used as fermentation substrate per garage fermenter and fermentation cycle, with straw representing 1% to 99% of the (fresh) fermentation mixture, preferably 50%. The garage fermenters are emptied and refilled with a wheel loader. The wheel loader needs 406 minutes (6.76 h) to empty and refill a garage fermenter. The fuel consumption of the wheel loader used in the biogas plant is 17.4 liters of diesel equivalent per operating hour and thus 117.6 liters of diesel equivalent per fermentation mass change. The energy input is about 1,153 kWh_{Hi} per fermentation mass change, of which the fermentation substrate, i.e. straw, accounts for about half, i.e. about 576 kWh_{Hi}. Using 60 t of straw-WM, 1 t of straw-WM accounts for 9.6 kWh_{Hi} and the calorific value of 1 kWh_{Hi} of input material corresponds to 0.0024 kWh_{Hi} or 0.24%. Since the wheel loader used has a CNG or LNG drive according to the invention and is operated with an absolutely GHG-free mixed gas produced according to the method of the invention, no GHG emissions arise from the operation of the wheel loader.

In total, the “pretreatment & fermentation” process step requires an energy input of 21.4 kWhₑₗ and 17.6 kWh_{Hi} per 1 ton of straw wet mass. The GHG emission associated with this energy input amounts to 21.4×540 gCO₂-eq/kWhₑₗ=11,556 gCO₂-eq/t straw-WM for the electricity input and to 0.0 gCO₂-eq/t straw-WM for the fuel input of the wheel loader.

In the anaerobic bacterial fermentation of steam pre-treated straw into biogas, the dry substance of the straw is converted into biogas in which methane makes up 53,50% by volume, carbon dioxide 44.60% by volume, hydrogen 0.15% by volume, oxygen 0.75% by volume, nitrogen 0.75% by volume, hydrogen sulfide 0.05% by volume and ammonia 0.20% by volume. The calorific value of the biogas is essentially determined by the methane content. With a methane yield of 2,860 kWh_{Hi}/t straw-WM (conversion efficiency 70%) and a specific calorific value of 9.978 kWh_{Hi}/Nm³ CH₄, a methane volume of 286.6 Nm³ and a total biogas volume of 535.7 Nm³ result. Methane has a share of 286.6 Nm³ in this biogas volume, carbon dioxide 238.9 Nm³, hydrogen 0.804 Nm³, oxygen 4.018 Nm³, nitrogen 4.018 Nm³, hydrogen sulfide 0.268 Nm³ and ammonia 1.071 Nm³.

Carbonization of the Straw-Containing Fermentation Residues

An essential element of the invention is to treat residues from a first biomass conversion into (marketable) energy carriers, preferably straw-containing conversion residues, in such a way that the atmospheric carbon still contained in these fermentation residues is at least partially chemically and physically stabilized. The first biomass conversion can be any biomass conversion that produces carbon-containing conversion residues (e.g. the conversion of biomass into bio-diesel, the conversion of biomass into bio-ethanol, the conversion of biomass into lingo-ethanol, the conversion of biomass into Fischer-Tropsch fuels, the conversion of biomass into hydrogen, the conversion of biomass into biogas and similar known processes).

The chemical-physical stabilization of the atmospheric carbon still contained in the residues of the first biomass conversion is preferably carried out in such a way that the atmospheric carbon does not react with other substances (atmospheric oxygen) and/or does not decompose with soil respiration (i.e. Is not decomposed by soil organisms) for decades, more preferably for centuries and in particular for millennia.

Preferably, chemical-physical stabilization is achieved by thermo-chemical treatment, which is more preferably carbonization and consists in particular of a selection of the following previously known carbonization processes: pyrolysis, torrefaction, carbonization, gasification, hydrothermal carbonization (HTC), vapothermal carbonization, any combination of these measures. Preferably, corresponding equipment known from the relevant prior art is used for this purpose.

Carbonization of the residues from the first biomass conversion preferably results in a biochar/vegetable coal/biocoke which contains at least proportionately stabilized or partially stabilized atmospheric carbon.

Preferably, the proportion of stabilized or partially stabilized atmospheric carbon in the dry substance of the biochar/vegetable coal/biocoke produced is more than 1%, more preferably more than 15%, in particular more than 45% and most preferably more than 70%.

In order to maximize the proportion of stabilized or partially stabilized atmospheric carbon in the dry substance of the biochar/vegetable coal/biocoke produced, the treated residues from the first biomass conversion are brought as slowly as possible to reaction temperature in a preferred embodiment of the invention. The heating up to reaction temperature therefore preferably takes longer than 1 second, in particular longer than 10 minutes and at best longer than 100 minutes.

Preferably, the residues from the first biomass conversion are divided into two, three or four partial streams, wherein the first partial stream is fed to pyrolysis, the second partial stream is treated by means of a selection of low-temperature pyrolysis, short-term pyrolysis, carbonization, gasification, torrefaction, HTC and vapothermal torrefaction and results in an at least partial stabilization of the carbon, the third partial stream is treated by a selection of low temperature pyrolysis, short term pyrolysis, torrefaction, HTC, carbonization, gasification and vapothermal torrefaction and does not result in carbon stabilization, and the fourth partial stream is not subjected to thermo-chemical treatment.

In a preferred embodiment, pyrolysis is a high-temperature pyrolysis, wherein the residues from the first biomass conversion are subjected to a temperature of 150° C.-1,600° C., preferably a temperature of 500° C.-1,000° C. and in particular a temperature of 600° C.-900° C., under oxygen deficiency.

Preferably, the reaction mass is exposed to the reaction temperature for more than 1 second, more preferably for more than 50 minutes and in particular for more than 500 minutes.

Preferably, the pressure in the reaction vessel used for the thermo-chemical treatment of the residues from the first biomass conversion corresponds to the pressure of the environment, more preferably >1 bar and in particular >5 bar.

In an advantageous embodiment of the invention, the conversion residues exposed to carbonization are available in the form of pellets or briquettes, while essentially retaining this form, preferably during carbonization, and/or the output of the carbonization devices being essentially in the form of pellets or briquettes.

In a preferred embodiment of the method according to the invention, a special two-, three- or four-part fermentation residue/vegetable coal mix consisting of fermentation residues and low- and high-temperature coals is produced and introduced into the arable soil as a substitute for straw removed from the field. While the carbon content of the untreated fermentation residue is not stabilized and the atmospheric carbon contained in the torrefied low-temperature vegetable carbon (or in the vegetable coal pyrolyzed at relatively low temperatures and/or only for a short time) has only moderate stabilization, the carbon fraction subjected to high-temperature pyrolysis is chemically stabilized after pyrolysis in such a way that, after incorporation into the arable soil, it cannot be degraded either by the process of soil respiration or by the process of aerobic rotting and consequently becomes part of the permanent humus. At the same time, the untreated fermentation residue and the torrefaction coal become part of the nutrient humus. In order to avoid adverse (short-term or temporary) immobilizations of nutrients in the soil, the fermentation residue/vegetable coal mix can first be enriched (loaded) with organic nutrients.

Preferably, the molar H/C ratio a) of the (partially) stabilized atmospheric carbon, b) of the produced, strongly C-containing biochars/vegetable coals/biocokes E to G, c) of the biochar/vegetable coal mixture H and/or d) of the biochar/vegetable coal conversion residue mixture I is <0.8, more preferably <0.6, and/or its molar O/C ratio thereof is <0.8, more preferably <0.4.

Such biochars/vegetable coals/biocokes or biochar/vegetable coal/biocoke mixtures or biochar/vegetable coal/biocoke conversion residue mixtures are chemically stabilized, at least in part, in such a way that the stabilized part of the atmospheric carbon contained in the biochars/vegetable coals/biocokes cannot be degraded within a given long-term period (10 years, 30 years, 100 years, 500 years, 1,000 years, 10,000 years, 100,000 years) by either the process of soil respiration or by the process of aerobic rotting or by reaction with atmospheric oxygen. It is therefore sufficient to store the produced biochars/vegetable coals/biocokes protected from the weather in order to remove carbon from the atmosphere of the earth and bring about a decarbonization of the atmosphere of the earth. Such storage can include, for example, the storage of produced biochars/vegetable coals/biocokes in storage halls (loose or in bogs), in abandoned mines, in caverns protected from the weather, in quarries under a protective layer or cover, in bogs, in deserts under a layer of sand, in the ocean floor under a layer of mud, in stagnant waters under a layer of mud or in aquifers.

Due to the above described positive effects of biochar/vegetable coal/biocoke on agricultural soils, in particular on field topsoil, it is advantageous to incorporate the biochar/vegetable coal or the biocoke produced according to the method of the invention at least partially into agricultural soils, in particular into field topsoil, either alone or together with non-carbonized residues from single-stage or multi-stage biomass conversion. For the removal of atmospheric carbon from the atmosphere of the earth, however, its chemical-physical stabilization and weather-resistant storage is sufficient in Itself, so that to achieve the desired GHG effect it is not absolutely necessary to incorporate the biochar/vegetable coal/biocoke into agricultural or forestry soil or to store it in caverns, quarries, desert soils, permafrost soils, aquifers, oceans, etc., in order to decarbonize the atmosphere of the earth.

The amount of short-term, medium-term and long-term stabilized atmospheric carbon entering the field topsoil is greater by a factor of 1.5-5.7 than if the farmer had left all the straw on the field and greater by a factor of 2.0-8.2 than if he had removed 30% of the straw growth from the field in accordance with good practice. Although the straw is removed from the field with a maximum (future) share of up to 87% in a particularly preferred embodiment of the method according to the invention, and although (gas) fuel is produced from this straw, the method is considerably better for the soil quality and the farmer than if the straw removal had not taken place. Despite this maximized recovery rate of up to 87%, the method achieves high and very high humus effects with the carbonization combination consisting of short- and medium-term humification to nutrient humus and long-term sequestering of the stabilized vegetable coal, resulting in a whole series of positive secondary effects which are listed above.

As a result, the user of the method according to the invention or the system according to the invention can very advantageously access the complete proportion of straw house growth, which up to now had to remain on the field in addition to the unrecoverable straw proportion (stubble, chaff, husk), in order to secure the level of the humus content of the arable soil. This makes all previous calculations to determine the biomass potentials of residual and waste materials for sustainable energetic utilization obsolete, including the study “Biomossepotenziale von Rest- und Abfallstoffen-Status Quo in Deutschland [Biomass potentials of residual and waste materials—status quo in Germany]”, recently prepared by the German Biomass Research Center (GBRC) and published by the Agency for Renewable Resources (ARR).

Taking into account an increase in straw growth to a total of about 46 million tons and a future requirement for bedding and roughage of about 4 million tons, the method/system according to the invention increases the amount of straw that can be used for energy and material purposes by a factor of 4.3 from the previous figure of about 8 million tons to about 34.1 million tons without endangering the humus content of the arable soil and the sustainability. Accordingly, the method/system according to the invention can produce significantly more advanced biofuel from the German straw growth than all other straw conversion processes, namely up to 100,000 GWh_{Hi} (360 PJ) without the addition of natural gas and up to 125,000 GWh_{Hi} (450 PJ) with the addition of natural gas. Without the addition of natural gas, the fuel quantity of 100,000 GWh_{Hi} would be strongly GHG negative and with the addition it would be GHG neutral.

These gas quantities increase significantly if not only straw but also farm manure and/or leaf waste are used in the method/system according to the invention.

In order to feed the straw-containing fermentation residues from the garage fermenters to the preferred sub-processes of pyrolysis and/or torrefaction, the water content (dry substance) of the fermentation residues must be reduced (increased) from about 65% (35% DS) to at least 50% (50% DS). This dehydration can be carried out in two steps according to the method of the invention, first by solid/liquid separation using a decanter/screw press to a DS content of up to 40% and then to a DS content of 50-70% by means of drying, which is preferably low-temperature drying. In contrast to high-temperature drying, neither (toxic) dioxins nor furans are produced during the drying process. The energy required for drying is obtained by the drying plant preferably on the basis of a process heat recirculation from the high-temperature pyrolysis.

With a biogas conversion efficiency of 70%, about 483 kg are obtained as more or less wet fermentation residue per 1 ton of straw wet mass. It has a dry substance content of 35% (169 kg) and a water content of about 65% (314 kg), wherein 140 kg of water was contained in the supplied native straw and the straw additionally absorbed about 174 kg of water in the process steps of pretreatment, mixing with other fermentation substrates and during fermentation. Carbon has a share of up to 134 kg in the DS content of about 169 kg. Of the 860 kg DS introduced into the fermenter, 691 kg was converted into biogas by the microorganisms.

The solid/liquid separation by phase separation by decanter/screw press is carried out to a DS content of 40%. Neglecting the fact that the liquid phase also has a certain DS content which cannot be filtered out (small straw particles, dissolved minerals, acids and salts), the straw-containing fermentation residue is reduced from 483 kg to 423 kg wet mass, which consists of 169 kg dry substance and 254 kg water. This releases 483-423=60 kg of a suspension containing small straw particles, dissolved minerals, acids and salts (the more liquid phase), which are added to the percolate as process water. This first dehydration by phase separation has a power requirement of 3.75 kWhₑₗ/t wet mass, in this case 0.483×3.75=1.81 kWhₑₗ/t straw-WM. For a future LCA-THG emission of the German electricity mix of 540 gCO₂-eq/kWhₑₗ, this results in a GHG emission of 1.81×540=978 gCO₂-eq/t straw-WM.

For every 1 ton of straw-WM that is fed to the biogas plant or the fermenter as a native fresh mass, the low-temperature drying plant produces a moist fermentation mass of 423 kg (the more solid phase), 169 kg of which consists of dry substance (134 kg of which is carbon) and 254 kg of which is water. In order to achieve a DS content of 60% (282 kg WM consisting of 169 kg DS and 113 kg H₂O), about 141 kg of water must be removed from the humid fermentation mass. This fermentation residue has a temperature of about 35° C. In order to reach the temperature of 100° C., the temperature must thus be raised by 65° C. With a heat capacity of 4,180 kJ per ton and ° C., precisely 0.423×65×4,180=114,929 kJ are required to heat the fermentation residue to 100° C., which corresponds to 31.9 kWh_{Hi}. The low-temperature drying plant obtains this energy from the waste heat generated during the pyrolysis or torrefaction of the dried fermentation residues.

The evaporation of the water to be removed from the fermentation residue (141 kg) requires a heat input of 2,088 kJ/kg, in this case 141×2,088=294,408 kJ, which corresponds to a total of 81.8 kWh_{Hi} and 0.58 kWh_{Hi} per kg of water. The low-temperature drying plant also obtains this energy from the waste heat generated during the pyrolysis or torrefaction of the dried fermentation residue.

In total, drying the dehydrated fermentation residue produced per 1 ton of straw wet mass to a DS content of 60% requires a heat supply of 31.9 kWh_{Hi}, +81.8 kWh_{Hi}=113.7 kWh_{Hi}. Since the drying plant is operated with waste heat from fermentation residue pyrolysis or torrefaction, there are no additional GHG emissions because the original input material is straw, the carbon of which originates from the atmosphere.

The about 100° C. hot, straw-containing fermentation residue stream from the low-temperature drying process can be divided into a first partial stream which is not treated, a second partial stream which is subjected to a weak torrefaction, a third partial stream which is subjected to a strong torrefaction and a fourth partial stream which is pyrolyzed. The partial streams can each have a share of 0%-100% of the total stream under the secondary condition that the sum of the partial streams does not exceed 100%. In the following, a division of the total stream into 3 partial streams is described as an embodiment; in other embodiments, however, a division into 1 partial stream, 2 partial streams and 4 partial streams is also possible.

After the straw-containing total fermentation residue stream has been divided into partial streams, the hot torrefied and pyrolyzed biochars/vegetable coals/biocokes are quenched with a selection of the following nutrient-containing aqueous suspensions: slurry, percolate, swill, stillage from ethanol production, liquid residues from anaerobic fermentation, urine, seepage water from silages, process water, treated or purified process water, liquid fermentation mass, permeate, more liquid phase of dehydration, more solid phase of dehydration, any phase of separation, suspensions containing other nutrients and similar suspensions (e.g. suspensions of water and mineral fertilizer), mixed with the untreated fermentation residue and, if necessary, subjected to a further low-temperature drying process to reduce the delivery mass of vegetable coal and/or for easier handling. The biochars/vegetable coals/biocokes produced can be quenched individually or as coal mixtures or coal conversion residue mixtures.

The carbonization of the straw-containing fermentation residue is preferably carried out in closed devices in which the gases released during the carbonization process (pyrolysis gases) are used in particular to heat the carbonization device (pyrolysis device).

Carbonization can take place in a parallel circuit and in a serial circuit. In the latter case, the carbonization devices are used for both torrefaction and pyrolysis. The only difference is the selected reaction temperature and reaction time, both of which are higher for pyrolysis than for torrefaction. In principle, however, other processes can also be used for carbonization, such as hydrothermal carbonization (HTC) or vapothermal carbonization.

Apart from the start-up process, a pyrolysis system does not require any heat input; on the contrary, it generates significantly more heat than is required for carbonization. At the current state of the art, the plant requires about 55.6 kWhₑₗ of electricity per ton of fuel-DS. It is foreseeable that the specific electricity consumption will decrease further with increasing size of these plants (economies of scale).

Based on the original straw input into the biogas plant or the fermenter (1 ton of straw-WM), the output from the low-temperature drying process is 282 kg of fermentation residue wet mass, of which 169 kg is DS and 113 kg is water. 134.2 kg of the 169 kg DS is carbon (see above). 6.67% of the dried fermentation residue (18.8 kg WM, of which 7.5 kg water, 11.3 kg DS, of which 8.9 kg C) is initially set aside in order to be added later to the vegetable coals produced with the aim of providing the soil flora and fauna with easily digestible biomass (OSS).

Another 6.67% of the 282 kg fermentation residue wet mass (18.8 kg WM, of which 7.5 kg water, 11.3 kg DS, of which 8.9 kg C) is fed into the self-igniting torrefaction system and is preferably carbonized at 250° C.-300° C. In this process about 25% of the dry substance is lost and with it about 25% of the carbon still present, i.e. about 2.8 kg DS and 2.2 kg C. This lost carbon has a calorific value of about 9.1 kWh_{Hi}/kg and a total calorific value of 20.2 kWh_{Hi}. 0.58 kWh_{Hi}/kg×7.5 kg=4.35 kWh_{Hi} are required for the complete evaporation of the water content, so that 15.85 kWh_{Hi} is available from the torrefaction for external purposes. This leaves 8.5 kg WM, 8.5 kg DS, of which 6.7 kg C, and 0.0 kg H₂O.

Thus, 86.7% of the 282 kg fermentation residue wet mass resulting from low-temperature drying remains for high-temperature pyrolysis, i.e. about 244 kg wet mass. Of these, 146 kg are DS and 98 kg are water. 116.4 kg of the 146 kg DS are carbon. In the self-fire pyrolysis, about 40% DS and thus about 40% of the carbon still present is lost in the embodiment here shown, i.e. about 58 kg DS and 46.6 kg C. This lost carbon has a calorific value of about 9.1 kWh_{Hi}/kg and a total calorific value of 419 kWh_{Hi}. 0.58 kWh_{Hi}/kg×98 kg=57 kWh_{Hi} are required for the complete evaporation of the water content, so that 362 kWh_{F} are available from the pyrolysis for external purposes. This leaves 88 kg WM, of which 88 kg DS, of which 70 kg C, and 0.0 kg H₂O.

In total, a process heat quantity of about 16 kWh_{Hi}+362 kWh_{Hi}=378 kWh_{Hi} is available for internal and/or external purposes per ton of straw-WM originally supplied. Of these, 114 kWh_{Hi} are required for drying the fermentation residue from 40% DS to 60% DS (see above). This leaves a heat quantity of 264 kWh_{Hi} available for other purposes.

After the fermentation residues have been set aside, torrefied and pyrolyzed, 8.9+6.7+70=85.6 kg C are still present, of which 8.9 kg C are not stabilized, 6.7 kg C are partially stabilized and 70 kg C are permanently stabilized. If these 70 kg of permanently stabilized carbon are worked into the arable soil, the atmosphere is permanently relieved of 70 kg×3.664=about 256,480 gCO₂. Based on the biogas production of 2,860 kWh_{Hi} (see above), this results in a specific GHG emission of −90 g CO₂/kWh_{Hi} gross (prior to consideration of the GHG emissions occurring in the other process steps).

Energy consumption: As the pyrolysis facilities generate the required heat themselves, there is no need for external heat. However, the electricity requirement is (still) considerable; 55.6 kWhₑₗ are required per t fuel-DS for the operation of the pyrolysis plant, which corresponds to 0.0556 kWhₑₗ/kg DS. The torrefaction of 11.3 kg DS (see above) therefore produces about 0.6 kWhₑₗ and the pyrolysis of 146 kg DS about 8.1 kWhₑₗ, i.e. In total 8.7 kWhₑₗ.

GHG emission: A future LCA-GHG emission of the German electricity mix of 540 gCO₂-eq/kWhₑₗ results in a GHG emission of 8.7×540=4,698 gCO₂-eq/t straw-WM. Based on the fuel quantity produced of 2,860 kWh_{Hi}, a specific GHG emission of +1.6 gCO₂-eq/kWh_{Hi} results.

Production of a Nutrient-Loaded Fermentation Residue/Vegetable Coal Mixture

The fermentation residue placed aside, the vegetable coal coming from the torrefaction and the output of the high-temperature pyrolysis are mixed together to form a high-quality biochar/vegetable coal mixture with C-containing humus. To ensure that the fresh biochar/vegetable coal mixture does not remove any nutrients from the soil after incorporation into the topsoil and immobilizes them, the biochar masses E to G produced are enriched with exactly the same organic nutrients as those contained in the cereal plant according to the method according to the invention before or after mixing them into a biochar mixture H. The biogas plant obtains these nutrients from the percolate, which is produced inter alia from straw in the first partial step of anaerobic bacterial fermentation—hydrolysis—in the garage fermenter in the upstream process step of fermentation. This means that the straw constituents, which are organic nutrients except for the ash content, are at least partly washed out of the straw and collect in the percolate. The enrichment of the biochar/vegetable coal mixture with organic nutrients is preferably carried out by quenching with a selection of the following nutrient-containing aqueous suspensions: slurry, percolate, swill, liquid residues from anaerobic fermentation, stillage from ethanol production, urine, seepage water from silages, process water (possibly treated or purified), liquid fermentation mass, permeate, more liquid phase of dehydration, more solid phase of dehydration, any phase of separation, suspensions containing other nutrients and similar suspensions. If the amount of heat contained in the hot biochar/vegetable coal/biocoke is less than the amount of heat required to evaporate the supplied water (only the water evaporates during the quenching process, the organic nutrients dissolved in the water remain in the biochar/vegetable coal mixture), the biochar/vegetable coal mixture becomes wet again, otherwise it remains dry.

In order to prevent the formation of fungi and self-ignition and to reduce the transport weight of the biochar/vegetable coal mixture loaded with nutrients, the moist biochar/vegetable coal mixture is dried to a DS content of at least 86% in a second low-temperature drying plant, if necessary at all.

The stabilized biochar/vegetable coal/biocoke E, the partially stabilized biochar/vegetable coal/biocoke F, the unstabilized biochar/vegetable coal/biocoke G, the biochar/vegetable coal/biocoke mixture H composed of biochars/vegetable coals/bio-cokes E to G and the biochar/vegetable coal/biocoke conversion residue mixture I composed of the biochar/vegetable coal/biocoke mixture H and conversion residues D may or may not be individually loaded with organic nutrients. This means that it is possible and can be advantageous to load only individual components of these mixtures or mixtures with organic nutrients and not others. It is also possible to load all biochars/vegetable coals/biocokes E to G with organic nutrients and none. Furthermore, it is possible and can be advantageous to load all components of the biochar/vegetable coal/biocoke mixture H and the biochar/vegetable coal/biocoke conversion residue mixture I with organic nutrients and none.

The fermentation residue placed aside (18.8 kg WM, of which 7.5 kg water and 11.3 kg DS, of which 8.9 kg C), the vegetable coal coming from the torrefaction (8.5 kg WM, of which 0.0 kg water, 8.5 kg DS, of which 6.7 kg C) and the output of the high-temperature pyrolysis (88 kg WM, of which 0.0 kg water and 88 kg DS, of which 70 kg C) are mixed to about 115.3 kg WM, which consists of 7.5 kg water and 107.8 kg DS (DS content thus 93.5%), with 85.6 kg C still being contained in the DS. Of the 85.6 kg C, 8.9 kg C is not stabilized, 6.7 kg C is partially stabilized and 70 kg C is permanently stabilized.

While the fermentation residue put aside has a temperature of about 25°, the temperature of the vegetable coal coming from the torrefaction is about 250° C. and that of the output of the high-temperature pyrolysis is about 700° C. After mixing, the mixture still has a temperature of (18.8×25° C.+8.5×250° C.+88×700° C.)/115.3=about 557° C. This temperature is so high that the water contained in the fermentation residue (7.5 kg) evaporates. The mass of the mixture thus decreases from 115.3 kg by 7.5 kg to 107.8 kg and the temperature due to the evaporation of the water and as a result of an intermediate storage to about 350° C.

The aqueous suspension used for quenching (preferably a selection from the following nutrient-containing suspensions): slurry, percolate, swill, stillage from ethanol production, liquid residues from anaerobic fermentation, urine, seepage water from silages, (If necessary, treated or purified) process water, liquid fermentation mass, permeate, more liquid phase of a dehydration, more solid phase of a dehydration, any phase of a separation, other nutrients-containing suspensions and similar suspensions), which consists more preferably of percolate from the fermentation of straw-containing fermentation mass, can be concentrated beforehand at least partially by means of filtration, ultrafiltration and reverse osmosis, namely to an average DS content of about 5.0%. 1 kg aqueous suspension therefore consists of 50 g DS and 950 g water. In order to enrich the DS of the biochars/vegetable coals/biocokes E-G (107.8 kg) with 1.7 kg nutrient mix, 34.0 kg percolate consisting of 1.7 kg DS and 32.3 kg water are required. The wet mass of the biochars/vegetable coals/biocokes E-G “loaded” with nutrients thus adds up to a theoretical 107.8+34.0=141.8 kg, the corresponding dry substance to 107.8+1.7=109.5 kg and the theoretical water content to 0.0+32.3=32.3 kg. The carbon content remains at 85.6 kg C.

After quenching with aqueous percolate, the biochar/vegetable coal mixture still has a temperature of (107.8×350° C.+34.0×25° C.)/141.8=about 272° C. This temperature is so high that about half of the water contained in the percolate, i.e. 16.2 kg, evaporates.

While the dry substance of the biochar/vegetable coal mixture enriched with nutrients remains at 109.5 kg and the carbon content at 85.6 kg C, the wet mass decreases as a result of water evaporation from 141.8 kg by at least 16.2 kg to a maximum of 125.6 kg and accordingly the water content from 32.6 kg by at least 16.2 kg to a maximum of 16.2 kg. The DS content only decreases to 109.5/125.6=87.2% as a result of quenching, so that post-drying to the desired DS content of at least 86% can be omitted.

Energy input: The energy input required for mixing and post-treatment is so low that it can be neglected.

GHG emission: When fermentation residues are mixed with fresh vegetable coal and post-treated, virtually no GHG emissions occur.

Use of the Fermentation Residue/Vegetable Coal Mixture

After cooling to below 40° C., the biochars/vegetable coals/biocokes E to G loaded or not loaded with organic nutrients and the biochar/vegetable coal/biocoke mixtures H and I are placed in suitable silos, preferably in sacks, more preferably in so-called BigBags, and stored temporarily in this form. These biochars/vegetable coals/biocokes or biochar/vegetable coal/biocoke mixtures can be stored both in bulk and in the form of granulate, flour, crumbs, pellets or briquettes. It is also possible to store these biochars/vegetable coals/biocokes initially in bulk and granulate, shred, grind, grind, pelletize or briquette them shortly before they are transported to their customers.

If necessary, the BigBags are removed from the silos or the intermediate storage facility and delivered by truck, preferably with trucks that have CNG or LNG drive and use GHG-free gas fuel produced according to the method of the invention. To load the trucks, the fermentation residue/vegetable coal mixture loaded with organic nutrients and filled into BigBags is removed from the interim storage facility by means of a telescopic loader or crane and loaded onto trucks, preferably semi-trailers. The truck can also be equipped with a crane so that loading and unloading can also take place at locations where no suitable loading technology is available.

With an average delivery distance (distance from the vegetable coal interim storage facility of the biogas plant to the regional interim storage facility and/or to the farm) of 50 km and a load of 20 t of vegetable coal (bulk density 0.36 t/m³), the truck provides a transport capacity of 1,000 tkm per load. With a consumption of 33 liters of diesel equivalent per 100 km, an energy quantity of about 163 kWh_{Hi} is used for long-distance transport, and with a consumption of 28 liters for the empty journey back to the decentralized warehouse, another 137 kWh_{Hi} is used. The total energy consumption for long-distance transport therefore amounts to about 300 kWh_{Hi} per load and 15 kWh_{Hi} per ton of vegetable coal.

Since the delivery trucks will be equipped with CNG or LNG engines that refuel and use GHG-free mixed gas produced according to the method of the invention, transporting the vegetable coal to the customers does not cause any GHG emissions.

Based on the original input of 1 ton of straw wet mass, the method according to the invention can provide about 125.6 kg of “loaded” fermentation residue/vegetable coal mixture with a carbon content of 85.6 kg C (see above) at a biogas conversion efficiency of 70%. The delivery of this quantity requires a fuel amount of 15 kWh_{Hi}/t×0.1256 t=1.9 kWh_{Hi}. This energy input is not associated with the emission of greenhouse gases for the reasons given above.

The contents of the BigBags delivered are filled into a fertilizer spreader on the yard of the farm or at the edge of the field, together with mineral fertilizer if necessary, and spread alone or as usual together with the fertilizer on the field and then worked into the soil, preferably into the topsoil. With the exception of the negligible work involved in filling the fertilizer spreader, there is no additional work involved, as the farmer ploughs or uproots the topsoil anyway. Since this does not result in any additional energy expenditure, GHG emissions do not occur. The application of the loaded biochar/vegetable coal mixture can, of course, also take place without mixing with mineral fertilizer.

Alternatively, the fermentation residue/vegetable coal mixture, which can also consist only of vegetable coal or a vegetable coal mixture, can also be mixed with solid manure, e.g. when loading solid manure spreaders. The fermentation residue/vegetable coal mixture is then spread on the agricultural land together with the solid manure and worked into the soil.

In a further option, the fermentation residue/vegetable coal mixture, which can also consist only of vegetable coal or a vegetable coal mixture, can also be mixed with slurry or liquid fermentation residues—If necessary after previous comminution to a correspondingly fine size 13 e.g. before or after loading slurry transporters and/or slurry distributors. The fermentation residue/vegetable coal mixture is then spread together with the slurry or the liquid fermentation residues on the agricultural land and worked into the soil.

In contrast to the application of mineral fertilizer, solid manure or slurry, no laughing gas emissions are produced during the application of the fermentation residue/vegetable coal mixture; on the contrary, these emissions are reduced, so that this process step leads to a reduction in GHG emissions, the level of which, however, is still unknown. In order to determine the CO₂ equivalence of N₂O emissions, the high weighting factor of 298 must be applied. This means that even small reductions in N₂O emissions can lead to large reductions in GHG emissions quantified in CO₂ equivalents. On this basis, it appears justified to attribute or add these reductions in laughing gas emissions to those GHG emissions which may have been overlooked in the above description.

The incorporation of the fermentation residue-vegetable coal mixture produced according to the method of the invention into the arable soil ensures that the humus content is maintained and increased even during the complete removal of the harvestable straw. This means that the users of the method according to the invention also have access to the portion of straw that previously had to remain in the field to safeguard the humus content. Instead of only 8-13 million tons so far, up to 34 million tons can now be used to generate energy in Germany alone (46 million tons of straw growth×87% recoverable share 32 40 million tons./. 4 million tons of bedding 32 36 million tons).

Depending on the biogas conversion efficiency (controllable between 10% and 70%) without the addition of natural gas, the quantity of gas fuel that can be produced from this quantity of straw alone can reach 36,000,000×409=about 14,700 GWh_{Hi} (53 PJ) to 36,000,000×2,860=103,000 GWh_{Hi} (370 PJ). Including the first natural gas admixture (without GHG effect from CO₂ recuperation), up to 184,000 GWh_{Hi} (660 PJ) of absolutely GHG-neutral mixed fuel can be provided. With future annual fuel consumption expected to fall to about 3,000 kWh_{Hi} per passenger car (about 340 liters gasoline equivalent) as a result of drive hybridization, the method according to the invention and the system according to the invention alone will be able to supply up to 65 million gasoline car equivalents with zero-emission fuel from the future German straw growth—i.e. without straw imports.

Depending on what proportion of the atmospheric carbon contained in the straw is converted into biogas in accordance with a controllable conversion efficiency and what proportion of carbon remains in the fermentation residues of the anaerobic bacterial fermentation, the introduction of chemically stabilized biochar/vegetable coal or chemically stabilized biocoke into the arable soil also has a (controllable) GHG-negative sequestration or decarbonization effect, which can reach net −196 gCO₂-eq/kWh_{Hi} to −1,790 gCO₂-eq/kWh_{Hi} of the gas fuel produced (the term “net” in this context means that GHG emissions associated with cultivation, processing, transport and distribution are taken into account). According to the invention, this effect is used to add such an amount of fossil natural gas to the GHG-negative straw-gas produced from straw that a mixed gas is produced, the GHG emission value of which is exactly 0.0 gCO₂-eq/kWh_{Hi}. In this way, 0.7 to 6.6 kilowatt hours of natural gas can be mixed with GHG-negative straw-gas per kilowatt hour produced. The straw-based fuel quantity available for zero-emission vehicles increases by a factor of 1.7 to 7.6 as a result of this addition.

Treatment of Biogas to Straw-Gas and Carbon Doxide

When the German straw growth is used for the method according to the invention, considerable amounts of atmospheric CO₂ are produced during the processing of the biogas produced into straw-gas depending on the biogas conversion efficiency (biogas produced from straw according to the method of the invention typically consists of about 51.00% from methane, about 0.10% from hydrogen, about 0.20% from ammonia, about 0.05% from hydrogen sulfide, about 0.50% from oxygen, about 0.50% from nitrogen and about 47.65% from atmospheric CO₂). This atmospheric CO₂ can—as has been practiced since 2011 by the German company CropEnergies AG in the production of bio-ethanol—be liquefied and distributed to industrial customers, replacing fossil CO₂ previously used by them (e.g. In the food industry) or—as practiced by the Norwegian oil company StatOil—sequestered in the long term (e.g. as propellant gas in almost depleted oil reservoirs). In the first case, the increase in the amount of CO₂ in the atmosphere of the earth is prevented, in the second case atmospheric CO₂ is removed from the atmosphere of the earth. Both result in extended decarbonization effects recognized by the European Renewable Energy Directive (RED) 28/2009. Recuperated atmospheric CO₂ can, however, also be used as a basis for the production of synthetic energy carriers, such as synthetic methane, which according to Sabatler is produced from hydrogen and CO₂. If the hydrogen gas is produced by electrolysis from grid-decoupled RES electricity, such as wind power, the synthetic energy carrier is almost GHG-neutral. Except in the case of sequestration of the recuperated CO₂, the preparation or purification of the CO₂ is a prerequisite for application.

Atmospheric carbon dioxide (CO₂) produced as a by-product, waste or residual material when carrying out the method according to the invention is therefore preferably subjected to a selection from the following method steps: recuperation, purification, liquefaction, processing, sequestration (in geological formations, such as crude oil or natural gas deposits), substitution of fossil CO₂s, production of CO₂-based energy carriers (syn-methane, syn-methanol), any combination of these method steps as for as possible and reasonable. More preferably, the recuperated atmospheric CO₂ is sequestered in crude oil or natural gas deposits from which production is still taking place. In particular, the recuperated atmospheric CO₂ replaces (substitutes) fossil CO₂.

Based on the kilowatt hour of straw-gas produced, these further decarbonization effects result in a negative GHG value depending on the conversion efficiency. This value allows the further addition of fossil natural gas, resulting in a mixed gas, the GHG emission value of which is again 0.0 gCO₂-eq/kWh_{Hi} (compressed natural gas has a life cycle GHG emission value of 69.3 gCO₂-eq/MJ in the Otto external ignition engine (gas engine) in the EU mix according to EU Directive 2015/652 of 20 Apr. 2015, which corresponds to 249.5 gCO₂-eq/kWh_{Hi}).

Depending on the carbon content, which is converted into biogas (essentially CH₄ and CO₂) and the carbon content, which remains in the fermentation residues, as well as depending on the annual fuel consumption of a CNG cars (possibly equipped with hybrid technology), the method according to the invention and the system according to the invention can turn a large part of the German road vehicle fleet into genuine zero-emission vehicles from the German straw volume alone with a corresponding proportion of fossil natural gas, and at the same time considerably and sustainably improve the humus content of the German arable soils. Due to the size of this potential vehicle fleet, the technology disclosed here is clearly more than just a transitional technology for electric and hydrogen mobility.

The separation of biogas into methane and CO₂ is an established technology. In Germany alone, there are 200 biogas plants that process their biogas into bio-methane and feed it into the natural gas grid. Various processes are available for processing the biogas into bio-methane, wherein the energy required and the methane slip are different. There are several manufacturers offering such processes.

Cryogenic separation process: Cryogenic separation processes are advantageous for the method according to the invention because they allow the use of the CO₂ produced during biogas processing and the bio-methane produced has to be liquefied anyway during the production of the LNG substitute LBM (Liquefied bio-methane). In an advantageous embodiment of the invention, the CO₂ is therefore to be liquefied after purification and delivered in liquid form by truck to industrial customers. Since the CO₂ has a high mass share of about 70% in the biogas produced and should ultimately be present in the liquid state, it is advantageous to use a cryogenic process or plant to separate the carbon dioxide (sublimation point at −78.5° C.) and, if necessary, also the other gases (ammonia, hydrogen sulfide, oxygen, hydrogen) from the methane. They are offered by different manufacturers.

Ammonia has the highest boiling point of all gases contained in biogas with −33.3° C. at normal pressure. In a cryogenic separation process, ammonia is therefore the first to become liquid. Alternatively, the hydrogen sulfide can also be washed out of the biogas before the cryogenic separation using conventional technology.

Hydrogen sulfide (sulfuretted hydrogen) has the second highest boiling point at −60° C. at normal pressure. In a cryogenic separation process, hydrogen sulfide thus becomes the second liquid. Alternatively, the hydrogen sulfide can also be washed out of the biogas using conventional technology prior to cryogenic separation.

Carbon dioxide (carbon dioxide) has the third highest boiling or sublimation point at −78.5° C. at normal pressure. In a cryogenic separation process, CO₂ thus becomes the third liquid.

Methane, which boils at −161.5° C. at normal pressure, has the fourth highest boiling point. Methane is therefore the fourth liquid in a cryogenic separation process.

Since the remaining gases nitrogen, oxygen and hydrogen have only very small proportions in the biogas and are not harmful to the environment, they can be released into the environment after liquefaction of the methane and possibly after heat exchange with fresh biogas. However, they can also be used according to the relevant prior art.

If the biogas is purified in such a way that the hydrogen sulfide and the ammonia are first separated, the biogas pre-purified in this way is pressurized (1.1-50 bar, preferably 3-30 bar, in particular 5 to 15 bar and at best 6-8 bar) and gradually cooled. CO₂ becomes liquid at about −45° C. to −57° C. and can be discharged and, if necessary, at −162° C. also the methane if liquefied LBM is required. According to a generous calculation, the liquefaction of CO₂ requires an electricity input of 365 kWhₑₗ/t CO₂ or 0.365 kWhₑₗ/kg CO₂.

Assuming a conversion efficiency of 10% (instead of the 70% assumed above), 409 kWh_{Hi} of biogas are produced per ton of straw-WM originally used, based almost exclusively on the calorific value of the methane component. With a methane yield of 409 kWh_{Hi}/t straw-WM and a specific calorific value of 9.978 kWh_{Hi}/Nm³ CH₄, this results in a methane volume of 40.99 Nm³ and a total biogas volume of 80.37 Nm³. in this biogas volume, methane has a share of 40.99 Nm³, carbon dioxide 38.30 Nm³, hydrogen 0.08 Nm³, oxygen 0.40 Nm³, nitrogen 0.40 Nm³, hydrogen sulfide 0.04 Nm³ and ammonia 0.16 Nm³.

Assuming a conversion efficiency of 70%, the above and below quantities are higher or lower by a factor of 7.

Multiplied by the respective densities, the gas mass for methane is 40.99 Nm³×0.7175 kg/Nm³=29.41 kg, for carbon dioxide 38.30 Nm³×1.9770 kg/Nm³=75.71 kg, for hydrogen 0.08 Nm³×0.0899 kg/Nm³=0.01 kg, for oxygen 0.40 Nm³×1,4290 kg/Nm³=0.57 kg, for nitrogen 0.40 Nm³×1,2510 kg/Nm³=0.50 kg, for hydrogen sulfide 0.04 Nm³×1,4290 kg/Nm³=0.06 kg and for ammonia 0.16 Nm³×1,5359 kg/Nm³=0.25 kg. For the liquefaction of the CO₂ portion, which results from the fermentation of 1 ton of straw-WM —namely 75.71 kg CO₂—an electricity requirement of 75.71 kg×0.365 kWhₑₗ/kg CO_{2=27.6} kWhₑₗ results.

A future LCA-THG emission of the German electricity mix of 540 gCO₂-eq/kWhₑₗ results in a GHG emission of 27.6×540=14,922 gCO₂-eq/t straw-WM. Based on the fuel quantity of 409 kWh_{Hi} produced, this is 36.5 gCO₂-eq/kWh_{Hi}. At the same time, the substitution of atmospheric CO₂ for fossil CO₂ has a negative effect of −75,710 gCO₂/t straw-WM. Based on the 409 kWh_{Hi} of fuel produced, this is −185.1 gCO₂-eq/kWh_{Hi}.

The net effect is −75,710+14,922=−60,788 gCO₂-eq/t straw-WM. Based on the fuel quantity produced of 409 kWh_{Hi}, this is −148.6 gCO₂-eq/kWh_{Hi}.

Conventional biogas treatment: The impurities hydrogen sulfide and ammonia are first removed from the biogas produced from straw and co-substrates by anaerobic bacterial fermentation. The remaining gases, mainly methane and carbon dioxide, are then subjected to one of the established separation processes. This requires an electricity input of less than 0.15 kWhₑₗ/Nm³ crude biogas.

The crude biogas quantity per ton of straw-WM originally used is about 535.7 Nm³. Biogas upgrading therefore requires an electricity input of 535.7 Nm³×0.15 kWhₑₗ/Nm^{3=80.4} kWhₑₗ. A future LCA-THG emission of the German electricity mix of 540 gCO₂-eq/kWhₑₗ results in a GHG emission of 80.4×540=43,416 gCO₂-eq/t straw-WM. Based on the fuel quantity produced of 2,860 kWh_{Hi}, this is 15.2 gCO₂-eq/kWh_{Hi}.

The production of 2,840 kWh_{Hi} of biogas or methane produces about 472.4 kg CO₂ (2,860 kWh_{Hi} correspond to about 286.6 Nm³ methane with a specific methane calorific value of 9.978 kWh_{Hi}/Nm³; methane has a volume share of 53.5% of the biogas produced, so that the biogas quantity is 286.6 Nm³/0.535=535.8 Nm³. CO² has here a volume share of 44.60%, i.e. 238.9 Nm³. A density of 1.977 kg/Nm³ results in a CO² mass of 472.4 kg). Its liquefaction requires an electricity input of 472.4×0.365=172.4 kWhₑₗ. A future LCA-THG emission of the German electricity mix of 540 gCO₂-eq/kWhₑₗ results in a GHG emission of 172.4×540=93,110 gCO₂-eq/t straw-WM. Based on the quantity of fuel produced of 2,860 kWh_{Hi}, this is 32.6 gCO₂-eq/kWh_{Hi}. This value, which at first glance appears unfavorable, is put into perspective when one considers that the substitution of 472,400 gCO₂ represents the gross admixture of 472.400/249.5=1,893 kWh_{Hi} natural gas to the produced 2,860 kWh_{Hi} straw-gas (see below) or otherwise calculated the GHG value of the produced 2,860 kWh_{Hi} decreases by another 472,400/2,860=165.2 gCO₂-eq/kWh_{Hi}. The net effect of CO₂ capture and CO₂ substitution is therefore (165.2./.32.6) x−1=−132.6 gCO₂-eq/kWh_{Hi}.

The feeding of bio-methane into the national natural gas grid is an established technology; in Germany there are about 200 biogas plants that feed biogas processed into bio-methane into the natural gas grid. The energy input for the compression in transmission grids amounts to about 1% of the calorific value of the bio-methane quantity to be compressed.

With a conversion efficiency of 70%, the method according to the invention produces about 2,860 kWh_{Hi} of bio-methane per ton of straw input. Its compression for transmission through the national grid therefore requires an electrical power quantity of 28.6 kWhₑₗ. A future LCA-THG emission of the German electricity mix of 540 gCO₂-eq/kWhₑₗ results in a GHG emission of 28.6×540=15,444 gCO₂-eq/t straw-WM. Based on the produced fuel quantity of 1,634 kWh_{Hi}, this is 9.5 gCO₂-eq/kWh_{Hi}.

Adding Fossil Natural Gas to the Straw-Gas Fed into the Grid

According to the method of the invention, a quantity of fossil natural gas should be added to the produced GHG-negative bio-methane or the produced straw-gas such that a gas mixture results, the GHG balance of which is neutral or the GHG emission value of which is zero or the GHG emission reduction performance of which in comparison with the fossil reference (fuel base value) is exactly 100%—i.e. 94.1 kilograms of carbon dioxide equivalent per gigajoule or 338.76 gCO₂-eq/kWh_{Hi}. The amount added here depends on the GHG negativity (or negative GHG emission quantity) of the bio-methane produced.

Pressing the straw requires an energy input of 5 kWh_{Hi}/t straw-WM. According to the method of the invention, the tractors that pull the straw press have a CNG or LNG drive that uses the GHG-free mixed gas as fuel. Pressing the straw into square bales, therefore, does not involve GHG emissions.

Collecting the straw bales and loading the trucks with wheel loaders requires the use of 3.4 kWh_{Hi} of fuel per ton of wet straw mass. According to the method of the invention, the wheel loaders that collect and load the straw bales have a CNG or LNG drive that uses the GHG-free mixed gas as fuel. Collecting and loading the square bales is therefore not associated with GHG emissions.

Long-distance transport of straw by full trucks requires a fuel input of 15 kWh_{Hi}/t straw-WM. When conventional diesel fuel is used, 15 kWh_{Hi}×342.36 gCO₂-eq/kWh_{Hi}=5,134 gCO₂ eq would be emitted into the atmosphere per ton of straw-WM. However, since the method according to the invention provides that the trucks will be equipped with CNG or LNG engines that refuel and use GHG-free mixed gas produced according to the method of the invention, the long-distance transport of the straw does not cause GHG emissions either.

Since the straw is already chopped during the bale pressing process, this process step no longer has to be carried out in the biogas plant. This means that there is no energy input or GHG emissions in this respect.

Although the treatment of the straw with steam requires a heat input of about 8 kWh_{Hi}/t straw-WM transferred to the entire quantity of straw, this input is covered by process heat from the torrefaction or pyrolysis of the fermentation residues and is therefore not loaded with GHG emissions.

The conversion of the straw into biogas is associated with a generalized electricity input of 21.4 kWhₑₗ. In future, the German electricity mix will have a corresponding GHG load of 21.4 kWhₑₗ/t straw-WM×540 gCO₂-eq/kWhₑₗ=+11,556 gCO₂-eq/t straw-WM. The wheel loader causes an energy consumption of 9.6 kWh_{Hi} per ton of straw-WM. Since the wheel loader has a CNG or LNG drive and is operated with an absolutely GHG-free mixed gas produced according to the method of the invention, no GHG emissions result from the operation of the wheel loader.

The first dehydration of the straw-containing fermentation residues by phase separation has an electricity requirement of 1.81 kWhₑₗ/t straw-WM as described above. For a future LCA-THG emission of the German electricity mix of 540 gCO₂-eq/kWhₑₗ, this is a GHG emission of 8.07×540=+978 gCO₂-eq/t straw-WM.

Drying the dehydrated fermentation residue to a DS content of 60% requires a heat input of 81.8 kWh_{Hi}. Since the drying plant is operated with process heat (waste heat from fermentation residue pyrolysis or fermentation residue torrefaction), there are no additional GHG emissions.

The torrefaction and pyrolysis of the straw-containing fermentation residue, which occurs at an input of 1 ton of straw-WM, have an electricity requirement of 8.7 kWhₑₗ. A future LCA-THG emission of the German electricity mix of 540 gCO₂-eq/kWhₑₗ will result in a GHG emission of 8.7×540=+4,698 gCO₂-eq/t straw-WM.

The energy input required for mixing and loading fermentation residues with nutrients is so low that it can be neglected. When fermentation residues are mixed with fresh vegetable coal and loaded with plant nutrients, virtually no GHG emissions occur.

The delivery of the biochar/vegetable coal mixture H or the biochar/vegetable coal fermentation residue mixture I requires a fuel quantity of 1.90 kWh_{Hi} based on the input of 1 ton of straw-WM. For the reasons given above, this energy input is not associated with the emission of greenhouse gases.

Both the distribution of the biochar/vegetable coal mixture H or the biochar/vegetable coal fermentation residue mixture I on the field and its incorporation into the soil can take place together with mineral fertilizer, with farm manure (solid manure), with liquid fermentation residues from biogas production or with liquid fermentation residues (stillage) from bio-ethanol production. Consequently, no additional energy is required. With the incorporation of 70 kg of permanently stabilized carbon into the soil, these 70 kg C are removed from the atmospheric carbon cycle. The GHG effect converted into CO₂ equivalents is negative −256,480 gCO₂-eq (70 kg C×3.664) per 1 ton of straw input.

Biogas preparation to bio-methane requires an electricity input of about 80.4 kWhₑₗ per ton of straw input. A future LCA-THG emission of the German electricity mix of 540 gCO₂-eq/kWhₑₗ will result in a GHG emission of 80.4×540=+43,416 gCO₂-eq/t straw-WM.

The liquefaction of the recuperated CO₂ quantity of 472.4 kgCO₂/t straw-WM requires an electricity input of about 172.4 kWhₑₗ as explained above. A future LCA-THG emission of the German electricity mix of 540 gCO₂-eq/kWhₑₗ will result in a GHG emission of +93,110 gCO₂-eq/t straw-WM.

The substitution of fossil CO₂ by recuperated atmospheric CO₂ produces a GHG effect of −472,400 gCO₂/t straw-WM.

The compression of the produced straw-gas requires 28.6 kWhₑₗ of electrical power per ton of straw input. A future LCA-THG emission of the German electricity mix of 540 gCO₂-eq/kWhₑₗ will result in a GHG emission of +15,444 gCO₂-eq/t straw-WM.

In total, one ton of straw input results in the external energy input not covered by process energy of 328.3 kWhₑₗ and 34.9 kWh_{Hi}n. The GHG balance or total GHG emissions per ton of straw input are −559,678 gCO₂, which corresponds to −196 gCO₂-eq/kWh_{Hi} of bio-methane produced. As compressed natural gas (CNG) has a specific GHG emission of 69.3 kg CO₂-eq/GJ or 249.5 gCO₂-eq/kWh_{Hi} according to the EU directive, 559,678/249.5=2,243 kWh_{Hi} of CNG can be added to the strongly GHG-negative bio-methane in order to produce a mixed gas quantity of 2,860+2,243=5,103 kWh_{Hi}, the GHG emission of which is 0 gCO₂-eq. The GHG emission reduction performance is correspondingly 1,728,692 gCO₂-eq/t straw-WM or 100% or 338.76 gCO₂/kWh_{Hi} or 94.1 kg CO₂-eq/GJ.

The amount of natural gas added to the produced GHG-negative bio-methane increases in an advantageous way if not only straw is used as input material but also largely GHG-free input materials, such as slurry, sold manure, beet and potato haulm as well as legume waste.

This results in the admixture quantity of CNG resulting from the use of slurry, solid manure, beet and potato haulms as well as legume waste, as shown below. The embodiment here refers to the use of 60% of the national amount of slurry, sold manure, beet and potato haulms and legume waste. However, the degree of utilization can also be higher or lower.

If, in addition to the harvestable straw growth, 60% of the national amount of slurry, solid manure, beet and potato haulms and legume waste (a total of about 191 million tons of wet mass or 21.7 million tons of dry mass and about 10.0 million tons of atmospheric carbon) were used for the production of bio-methane, the method according to the invention could provide another 5,600-39,200 GWh_{Hi} (20-140 PJ) of gas fuel in addition to bio-methane produced from straw. Another 1.0-3.1 million t of chemically stabilized biochar/vegetable coal would be introduced into the arable land as permanent humus, with a further decarbonization effect of −3.8 million t CO₂-eq to −11.3 million t CO₂-eq. Based on the bio-methane quantity produced from slurry, manure, etc., this means a GHG value of −96 to −2,019 gCO₂/kWh_{Hi} gross and −61 to −1,984 gCO₂/kWh_{Hi} net. Credits for the prevention of slurry-based GHG emissions have not yet been taken into account.

This negative GHG effect allows the addition of 0.2-8.0 kWh_{Hi} of fossil natural gas per 1 kilowatt hour of bio-methane. Based on the used German slurry/manure/haulm volume (60%), about 10,000-45,000 GWh_{Hi} (36-160 PJ) of fossil natural gas could be added to the produced bio-methane without exceeding the GHG value of 0.0 gCO₂-eq/kWh_{Hi}. In total, the method according to the invention can provide up to 50,000 GWh_{Hi} (180 PJ) of zero emission fuel as an additional GHG-neutral mixed gas quantity through the use of slurry, solid manure and haulms, which covers the (gas) fuel requirement of another 17 million gasoline car equivalents with a future real driving annual fuel consumption of about 3,000 kWh per gasoline car and year.

Gas Transport in the National Gas Network & Availability at Gas Fling Stations Transport and removal of the energy equivalent of the mixed gas formed from straw-gas and natural gas from the natural gas grid does not require any effort since the distribution is usually only carried out virtually. The straw-gas molecules produced and the added natural gas are physically used at a completely different location than at the virtual exit point, i.e. usually near the entry point.

The energy equivalent of the mixed gas formed from straw-gas and natural gas is preferably supplied as a fuel, more preferably as a CNG or LNG substitute, to the end-consumers (motor vehicles with CNG or LNG drive). The compression of the gas removed from the natural gas grid to the 250 bar delivery pressure common at CNG filling stations requires an effort of 0.38 kWhₑₗ per cubic meter of natural gas, i.e. about 0.03 kWhₑₗ/kWh_{Hi}.

With a conversion efficiency of 70%, the method according to the invention produces 2,860 kWh_{Hi} bio-methane/t straw-WM. Due to the GHG negativity (or negative GHG emission quantity) of the produced gas fuel bio-methane, a CNG quantity of 2,243 kWh_{Hi} can be added, resulting in a mixed gas quantity of 5,103 kWh_{Hi}.

The compression thereof to 250 bar requires an electricity quantity of 5,103 kWh_{Hi}×0.03 kWhₑₗ=153.1 kWhₑₗ. A future LCA-THG emission of the German electricity mix of 540 gCO₂-eq/kWhₑₗ will result in a GHG emission of 153.1×540=82,669 gCO₂-eq/t straw-WM. Based on the produced fuel quantity of 5,103 kWh_{Hi}, this is 16.2 gCO₂-eq/kWh_{Hi}. To compensate for this GHG emission and to be able to offer an absolutely GHG-neutral mixed gas, it is only necessary to add 82.669/249.5=331 kWh_{Hi} of CNG less, i.e. instead of 2,243 kWh_{Hi} only 1,912 kWh_{Hi}. Thus a total of “only” 4,772 kWh_{Hi} per ton of straw input is available, which corresponds to 117% of the (lower) calorific value of this straw input.

Lie Cycle Greenhouse Gas Intensity of the Production Path According to the Invention

The bio-methane produced from straw is in any case GHG negative, no matter how high the biogas conversion efficiency is. In the event that the above calculated GHG emission values are higher or considered higher or that certain fuels (e.g. the gas fuel “mixed gas”) are replaced by other fuels (e.g. by conventional diesel fuel), the GHG negativity (or negative GHG emission quantity) of the produced bio-methane is reduced but without becoming GHG positive.

Straw, fuel and electricity are supplied as external energy carriers or energies to the system according to the invention with its system boundaries “straw collection” and “delivery of the energy equivalent gas fuel to end-consumers”.

The straw used has a (lower) calorific value of 4,750 kWh_{Hi} per ton of dry substance. With a usual dry substance content of 86%, this results in a calorific value of 4,085 kWh_{Hi} for 1 ton of straw wet mass. According to EU Directive 2009/28 (RED 1), the straw is not loaded with GHG emissions. The energies used for straw collection and straw harvesting is provided solely by the fuel used.

The fuel is required a) for the operation of the tractor, which pulls and drives the straw baler, b) for the wheel loader, which picks up the square bales with a multiple grab and loads them onto a truck, c) for the truck, which transports the straw to the biogas plant and returns empty, d) for the wheel loader which removes and refills the garage fermenters in the biogas plant and e) for the truck which transports the fermentation residue-vegetable coal mixture back to the straw supplier and returns empty.

The electric current is used a) for the conversion of straw into biogas (various systems of the biogas plants), b) for the dehydration of the fermentation residues (screw press or the like), c) for the torrefaction and pyrolysis of the fermentation residue (drive of the system), d) for the processing of the biogas into bio-methane, e) for feeding the bio-methane into the natural gas grid (compression), f) for liquefying the recuperated CO₂ and g) for the delivery to the end-consumer (compression to 250 bar). According to the German Federal Environment Agency, the so-called CO₂ emission factor for domestic electricity consumption is 615 gCO₂-eq/kWhₑₗ for 2013, 598 gCO₂-eq/kWhₑₗ for 2014 and 587 gCO₂-eq/kWhₑₗ for 2015. This factor is expected to decrease to 540 gCO₂ eq/kWhₑₗ by 2020, which is why this value is used here for calculations.

The inventor offsets the methane slip possibly occurring during the opening of the garage fermenters, fermentation mass change, biogas processing and delivery of the gas fuel to the end-consumer against the much higher GHG emission reduction performances that the method according to the invention achieves upstream through the energetic use of nitrogen-containing and very GHG-intensive farm manure (slurry, solid manure, poultry manure), the co-fermentation of which is advantageous in the garage fermenters already because it helps to achieve the C/N ratio of 20-40 (If the recirculation of N-containing percolate or process water is used) or this C/N ratio can be produced by a corresponding amount of farm manure alone.

The (lower) calorific value of the straw input is 4,085 kWh_{Hi}/t straw-WM, regardless of the subsequent conversion efficiency. The (lower) calorific value of the fuel input amounts to a total of 34.9 kWh_{Hi}. In the final expansion stage of the system according to the invention (option A), the tractor, trucks and wheel loaders have CNG or LNG drives as described above and use the GHG-free mixed gas produced. The GHG emission associated with the fuel input is therefore 0 gCO₂-eq/t straw-WM.

In option B, the tractor, trucks and wheel loaders have conventional diesel drives and use pure bio-diesel (FAME). According to the German Federal Agency for Agriculture and Food (FAF), FAME has only been loaded with 24.62 tCO₂-eq/TJ since 2015 and without iLUC, which corresponds to 88.6 gCO₂/kWh_{Hi} and a GHG emission reduction performance of 70.62%. The GHG emission associated with the fuel input thus amounts to 34.9×88.6=3,092 gCO₂-eq/t straw-WM.

In option C, the tractor, trucks and wheel loaders have dual fuel drives and use 80% B7 diesel, which consists of 7% FAME and 83% mineral diesel, and 20% GHG-free mixed gas. Since 2015, FAME has only been loaded with 88.6 gCO₂/kWh_{Hi} (see above), mineral diesel with 95.1 gCO₂-eq/MJ according to EU Directive 2015/652, which corresponds to 342.4 gCO₂-eq/kWh_{Hi}. B7 diesel thus has a GHG emission value of 324.6 gCO₂-eq/kWh_{Hi}. The use of 20% GHG-free mixed gas reduces the GHG emission to 342.4×0.8=259.7 gCO₂-eq/kWh_{Hi}. The GHG emission associated with the fuel input thus amounts to 34.9×259.7=9,064 gCO₂-eq/t straw-WM.

The electricity input, including the electricity input for the compression of the mixed gas consisting of bio-methane and natural gas, amounts to a total of 481.4 kWhₑₗ. This electricity consumption will cause a GHG emission of 481.4×540=259,956 gCO₂-eq/t straw-WM in 2020.

In total, option A generates GHG emissions of 259,956 gCO₂ eq per ton of straw input (wet mass). Based on the output of 2,860 kWh_{Hi} this is 90.9 gCO₂/kWh_{Hi}. In option B, GHG emissions increase by 3,092 g to 263,048 gCO₂-eq/t straw-WM and in option C by 9,064 g to 269,020 gCO₂-eq/t straw-WM. Based on the output of 2,860 kWh_{Hi}, this is 92.0 or 94.1 gCO₂/kWh_{Hi}.

The method according to the invention overcompensates these GHG emissions with the sequestration of atmospheric CO₂ and/or with the replacement of fossil CO₂ used in industry (e.g. In the food industry) by atmospheric CO₂ recuperated in the biogas plant. The incorporation and sequestration of the permanently stabilized carbon content in the fermentation residue-vegetable coal mixture has a negative GHG effect of −256,580 gCO₂-eq/t straw-WM. The GHG effect of the substitution of fossil CO₂ by atmospheric CO₂ is −472,400 gCO₂-eq/t straw-WM. Taken together, these two measures result in a decarbonization effect of −728,980 gCO₂-eq/t straw-WM.

In option A, the life cycle THG balance or the life cycle THG emission quantity is therefore −728,980+259,956=−469,024 gCO₂/t straw-WM. Based on the direct fuel production of 2,860 kWh_{Hi}, a specific GHG emission factor of −164 gCO₂-eq/kWh_{Hi} results. Option B leads to a “load” of −728,980+263,048=−465,932 gCO₂/t straw-WM. Based on the direct fuel production of 2,860 kWh_{Hi}, a specific GHG emission factor of −163 gCO₂-eq/kWh_{Hi} results. Option C leads to an environmental relief of −728,980+269,020=−459,960 gCO₂/t straw-WM. Based on the direct fuel production of 2,860 kWh_{Hi}, a specific GHG emission factor of −161 gCO₂-eq/kWh_{Hi} results.

These GHG-negativities (or the negative GHG-emission quantities) allow the admixture of fossil and GHG-positive natural gas. If the absolute value of the GHG emission associated with this natural gas admixture is the same as the absolute value of the GHG negativity determined above for bio-methane, a mixed gas is formed, the specific GHG emission factor of which is 0.0 gCO₂-eq/kWh_{Hi}. The invention thus produces a zero emission fuel despite the use of a fossil energy source.

The natural gas admixture quantity in option A is 469,024/249.5=1,880 kWh_{Hi}, in option B 465,932/249.5=1,867 kWh_{Hi} and in option C 459,960/249.5=1,844 kWh_{Hi}.

The result is a zero emission fuel production based on the input of 1 ton of straw wet mass of 2,860+1,880=4,740 kWh_{Hi} (option A), 2,860+1,867=4,727 kWh_{Hi} (option B) and 2,860+1,844=4,704 kWh_{Hi} (option C).

The lifecycle greenhouse gas intensity of the production path according to the invention for a mixed gas composed of GHG-negative bio-methane and GHG-positive natural gas therefore always has the value 0.0 gCO₂-eq/kWh_{Hi} or 0.0 gCO₂-eq/MJ.

Depending on the biogas conversion efficiency with which the bio-methane is produced from straw, it has a different GHG negativity (or different negative GHG emission values) before it is mixed with natural gas: bio-methane produced with a BG conversion efficiency of 10% has a life cycle greenhouse gas intensity of −1.635 gCO₂-eq/kWh_{Hi}, bio-methane produced with a BG conversion efficiency of 40% has a life cycle greenhouse gas intensity of −329 gCO₂-eq/kWh_{Hi} and bio-methane produced with a BG conversion efficiency of 70% has a life cycle greenhouse gas intensity of about −164 gCO₂-eq/kWh_{Hi}.

The GHG effect resulting from another evaluation of individual influencing factors, which cannot eliminate the GHG negativity but only reduces it, can therefore be cured by the systematics of the invention by simply adding a smaller amount of GHG-positive natural gas (CNG) to the produced GHG-negative bio-methane. This reduces the available amount of (true) zero emission fuel (mixed gas) but the GHG emission quantity of the zero emission fuel still remains at 0 gCO₂-eq/kWh_{Hi} or 0 gCO₂-eq/MJ.

These unusually good values are essentially based on the fact that atmospheric carbon is chemically and physically stabilized and sequestered (in the arable soil) and, in addition, carbon dioxide produced as a by-product is recuperated and fossil CO₂ is substituted.

Further Advantageous Embodiments of the Invention

The method according to the invention and the system according to the invention can be further improved by the recuperation, purification and liquefaction of atmospheric CO₂ resulting from the carbonization of the residues from single-stage or multi-stage biomass conversion, preferably during the pyrolysis and/or torrefaction of these conversion residues. The available amount of atmospheric CO₂ therefore increases substantially and thus the amount of atmospheric CO₂ available for fossil CO₂ substitution, the amount of atmospheric CO₂ available for sequestration and the amount of atmospheric CO₂ available for the production of CO₂-based fuels (heating mediums, combustion materials). Accordingly, the GHG negativity is further improved and with it the amount of available GHG-neutral mixed gas—after the addition of an additional amount of natural gas.

Alternatively, the recuperation, purification and liquefaction of CO₂ resulting from the carbonization of the residues from the single-stage or multi-stage biomass conversion, preferably during pyrolysis and/or torrefaction, can, of course, also be carried out alone without the recuperation, purification and liquefaction of CO₂ resulting from the processing of biogas into bio-methane.

In a preferred embodiment, the invention is based on an anaerobic bacterial fermentation of straw into biogas and the processing thereof into bio-methane. It goes without saying, however, that the method according to the invention and the system according to the invention can also be based on the fermentative production of ethanol from biomass, in particular on the production of ligno-ethanol from straw or wood, as well as on the production of other energy carriers from biomass, e.g. on the production of FT-fuels (syn-diesel, syn-gasoline, DME, bio-methanol, etc.) from straw or wood, of bio-diesel from rapeseed or palm oil, of hydrogen from biomass, etc. This shall comprise all biomass conversion methods, with which a person skilled in the art is familiar from the relevant prior art and/or from practice.

In a preferred embodiment of the invention, the single-stage or multi-stage biomass conversion consists of an anaerobic bacterial fermentation of fermentation substrate into biogas, an alcoholic fermentation of biomass into bio-ethanol, an (enzymatic) conversion of biomass into ligno-ethanol, a conversion of biomass into bio-diesel or FAME or HVO (e.g. transesterification), a conversion of biomass into Fischer-Tropsch fuels, a conversion of biomass into hydrogen (e.g. steam reforming), a conversion of biomass into bio-methanol (methanol synthesis) or a conversion of biomass into DME (DME synthesis).

An advantageous embodiment of the invention comprises a method in which process water is obtained from the course of a method step, preferably from the process of an anaerobic fermentation or alcohol fermentation, more preferably by a selection of the following processes: separation, decantation, pressing, filtration, ultrafiltration, reverse osmosis, heating, evaporation, concentration, sedimentation, crystallization, catalysis, phase separation, addition or use of polymers, any combination of these processes.

Another advantageous embodiment of the invention is the purification or processing of process water obtained from the process before it is reused with a selection from the following processes: separation, decantation, pressing, filtration, ultrafiltration, reverse osmosis, heating, evaporation, concentration, sedimentation, crystallization, catalysis, phase separation, addition or use of polymers, any combination of these processes. The reuse of purified process water is known to reduce the cost of water consumption for any water-using biomass conversion.

Another advantageous embodiment of the invention is that in the case of anaerobic (bacterial) fermentation, the average hydraulic residence time of the input material in the fermenter is less than 250 days, preferably less than 120 days, more preferably less than 70 days, in particular less than 40 days and at best less than 20 days. It is well known that low HRTs reduce the specific plant and capital costs per unit of energy (kWh_{Hi}, MJ) of each biomass conversion.

In a further advantageous embodiment of the method according to the invention, process heat from a process step is returned to the process, preferably from a thermal treatment of the biomass or the conversion residue into a warming or heating step, more preferably from a thermal treatment of the biomass before its conversion into a GHG-emission-reduced energy carrier, and in particular process heat from the chemical-physical stabilization of the atmospheric carbon. Preferably, the process heat is recirculated by means of a countercurrent heat exchanger, more preferably by means of water, in particular by means of pressurized water and/or steam, and at best by means of process water.

If the method according to the invention is applied, the atmospheric carbon mass of the biochar/vegetable coal/biocoke sequestered in the soil can decrease by less than 40% after 5 years by the process of soil respiration or by other chemical-physical processes, preferably by less than 10%, more preferably by less than 5% and in particular by less than 1%; or, by extrapolation, the mass of atmospheric carbon of the biochar/vegetable coal/biocoke sequestered in the soil decreases by less than 50% after 50 years by the process of soil respiration or by other chemical-physical processes, preferably by less than 25%, more preferably by less than 10% and in particular by less than 5%; or, by extrapolation, the mass of atmospheric carbon of the biochar/vegetable coal/biocoke sequestered in the soil decreases by less than 60% after 100 years by the process of soil respiration or by other chemical-physical processes, preferably by less than 30%, more preferably by less than 15% and in particular by less than 6%; or, by extrapolation, the mass of atmospheric carbon of biochar/vegetable coal/biocoke sequestered in the soil is reduced by less than 70% after 500 years by the process of soil respiration or by other chemical-physical processes, preferably by less than 50%, more preferably by less than 25% and in particular by less than 10%; or, by extrapolation, the mass of atmospheric carbon of biochar/vegetable coal/biocoke sequestered in the soil is reduced by less than 80% after 1000 years by the process of soil respiration or by other chemical-physical processes, preferably by less than 60%, more preferably by less than 30% and in particular by less than 15%; or, by extrapolation, the mass of atmospheric carbon of biochar/vegetable coal/biocoke sequestered in the soil is reduced by less than 90% after 10000 years by the process of soil respiration or by other chemical-physical processes, preferably by less than 70%, more preferably by less than 40% and in particular by less than 20%; or, by extrapolation, the mass of atmospheric carbon of the biochar/vegetable coal/biocoke sequestered in the soil is reduced by less than 95% after 100,000 years by the process of soil respiration or by other chemical-physical processes, preferably by less than 75%, more preferably by less than 50% and in particular by less than 25%.

In another advantageous embodiment of the method according to the invention and the system according to the invention, fly ash and dust are removed from the highly CO₂-containing flue gas from pyrolysis or torrefaction of the residues from the single-stage or multi-stage biomass conversion using the cyclone technology. Preferably, the resulting exhaust gas is freed from gaseous salts by means of an “acid gas scrubber” and fed to a CO₂ separation plant, which is preferably a cryogenic separation plant.

In a further advantageous embodiment of the method according to the invention, this method is designed in such a way that, compared to the greenhouse gas emission of the respective fossil reference fuel, heating medium or combustion material, the resulting GHG emission reduction performance of the generated fuel, heating medium or combustion material reaches a relative value between 1% and 10,000%, preferably a value between 5% and 5,000%, more preferably a value between 50% and 500% and in particular a value between 80% and 200%, based on the same amount of energy.

According to the invention it is preferred that the biomass consists of at least a portion of lignocellulose-containing biomass, preferably at least a portion of straw, more preferably at least a portion of wood.

In another preferred embodiment of the method according to the invention, aerobic rotting (oxidation) of straw into carbon dioxide (CO₂) and water in the field or in the arable soil is avoided by the straw utilization, conversion residue treatment and/or utilization of the resulting biochar/vegetable coal/biocoke, which consists largely of atmospheric carbon, at least partially, preferably at least 0.1%, more preferably at least 10%, in particular at least 50% and at best at least 80%, so that fewer greenhouse gases, in particular less CO₂, N₂O and CH₄, are formed in the (agriculturally used) soil than if the straw remained on the agriculturally used fields or would have been incorporated into the arable soil.

In another preferred embodiment of the method according to the invention and the system according to the invention, energy is used in the production, distribution and use of the energy carriers produced (fuels, heating mediums or combustion materials), in C-stabilization and in the incorporation of the produced biochar/vegetable coals mixtures and blends in such a way that energy carriers are used the GHG balance or GHG emission values of which are reduced compared to their fossil reference, more preferably to 0.0 gCO₂-eq/kWh or 0.0 gCO₂-eq/MJ and in particular are GHG-negative, i.e. have a negative GHG emission value.

In a further advantageous embodiment, the biogas plant described by US2006/0275895A1 (Jensen & Jensen) and the method disclosed therein for producing biogas from straw and/or straw-containing input materials are linked with at least one of the embodiments of the present invention, preferably with the chemical-physical stabilization of atmospheric carbon according to the invention, more preferably with the production of multi-part biochar/vegetable coal/biocoke mixtures or biochar/vegetable coal/biocoke conversion residue mixtures according to the invention and in particular with the incorporation of such biochar/vegetable coal/biocoke mixtures or biochar/vegetable coal/biocoke conversion residue mixtures according to the invention into agricultural soils.

In another advantageous embodiment, the biogas plant described by the inventor in EP 2167631B1 (Feldmann) and the method for producing biogas from lignin-containing renewable raw materials, which is disclosed therein, are combined with at least one of the embodiments of the present invention, preferably with the chemical-physical stabilization of atmospheric carbon according to the invention, more preferably with the production of single-part or multi-part biochar/vegetable coal/biocoke mixtures or biochar/vegetable coal/biocoke conversion residue mixtures according to the invention and in particular with the incorporation of such biochar/vegetable coal/biocoke mixtures or biochar/vegetable coal/biocoke conversion residue mixtures according to the invention into agricultural soils.

In a further advantageous embodiment, the method disclosed by EP2183374B1 (Fraunhofer) for the conversion of biomass from renewable raw materials into biogas in anaerobic fermenters is combined with at least one of the embodiments of the invention, preferably with the chemical-physical stabilization of atmospheric carbon according to the invention, more preferably with the production of single-part or multi-part biochar/vegetable coal/biocoke mixtures or biochar/vegetable coal/biocoke conversion residue mixtures according to the invention and in particular with the incorporation of such biochar/vegetable coal/biocoke mixtures or biochar/vegetable coal/biocoke conversion residue mixtures according to the invention into agricultural soils.

In a further advantageous embodiment, the method published under the application file number EP13807989.2 (Verbio) and the biogas plant for the production of biogas from lignocellulose-containing biomass, preferably from straw, which is disclosed therein, are Inked with at least one of the embodiments of the invention, preferably with the chemical-physical stabilization of atmospheric carbon according to the invention, more preferably with the production of single-part or multi-part biochar/vegetable coal/biocoke mixtures or biochar/vegetable coal/biocoke conversion residue mixtures according to the invention and in particular with the incorporation of such biochar/vegetable coal/biocoke mixtures or biochar/vegetable coal/biocoke conversion residue mixtures according to the invention into agricultural soils.

In a further advantageous embodiment, the method described by the inventor in European patent application EP12729875.0 (Feldmann) and the plant for producing atmospheric carbon dioxide from non-fossil energy carriers by means of the conversion processes of combustion, gasification and fermentation, which is disclosed therein, are Inked with at least one of the embodiments of the present invention, preferably with the chemical-physical stabilization of atmospheric carbon according to the invention, more preferably with the production of single-part or multi-part biochar/vegetable coal/biocoke mixtures or biochar/vegetable coal/biocoke conversion residue mixtures according to the invention and in particular by the incorporation of such biochar/vegetable coal/biocoke mixtures or biochar/vegetable coal/biocoke conversion residue mixtures according to the invention into agricultural soils.

According to various embodiments, the method according to the invention and the system according to the invention, can produce greenhouse gas-reduced energy carriers, preferably greenhouse gas emission-reduced biogas and/or greenhouse gas emission-reduced bio-methane, more preferably greenhouse gas-negative energy carriers and in particular greenhouse gas-negative biogas and/or greenhouse gas-reduced bio-methane.

### 7. BRIEF DESCRIPTION OF THE DRAWINGS

The figures show embodiments and details of the invention, wherein the basic concept of the invention and the scope of protection should not be restricted to the exact forms or details of the embodiments shown and described below. The scope of protection shall also include modifications (extensions and restrictions) which are previously known in the relevant arts and/or obvious to a person skilled in the art.

FIG. 1 shows a schematic diagram of a complex embodiment of the method and system according to the invention, in which a biomass is selected from many available biomasses, harvested and pre-treated prior to the (first) conversion, wherein the energy carrier generated with the (first) conversion is mixed with other energy carriers, the conversion residue is divided into four partial streams A to D and the conversion residues A to C are dehydrated, wherein the dehydration can be associated with nutrient extraction and releases nutrient-containing process water; after comminution and, if necessary, pelletization of the conversion residues possibly treated in this way, conditions for a chemical-physical stabilization of atmospheric carbon are created in a reaction vessel; as soon as these conditions exist, the atmospheric carbon contained in conversion residues A to C is chemically and physically stabilized, namely A is fully stabilized, B is partially stabilized and C is not stabilized; this (partial) stabilization results in biochar masses E to G, which are quenched with the nutrient-containing process water and loaded with these nutrients; the biochar masses are mixed with one another to form a biochar mixture H which is mixed with untreated conversion residues D to form a biochar conversion residue mixture I; after pelletizing the biochar mixture H or the biochar conversion residue mixture I, the pellets produced are filled into BigBags and distributed via regional interim storage facilities to the farms where they are filled into solid manure spreaders, fertilizer spreaders or slurry distributors. After the application of the biochar mixture or the biochar conversion residue mixture, these are worked into the topsoil of the fields where they can develop their positive effect on the soil and the atmosphere of the earth.

FIG. 2 shows a further schematic diagram of an embodiment of the method and system according to the invention without the conversion residue D indicated in FIG. 1 and with a first recuperation of atmospheric carbon dioxide (CO₂ I) on the occasion of the (first) conversion of the selected biomass into a produced energy carrier; furthermore, a second recuperation of atmospheric carbon dioxide (CO₂ II) is shown; CO₂ I and CO₂ II are combined, liquefied and geologically sequestered, used as substitutes for fossil CO₂ or used for the production of CO₂-based energy carriers.

### 8. DETAILED DESCRIPTION OF THE PREFERRED EMBODIMENTS

For a better understanding of the present invention, reference is made in the following to the embodiments shown in the drawings, which are described using specific terminology. The terminology is consistent, i.e. the respective designations apply to all figures. It should be noted that the scope of protection of the invention shall not be restricted by the designation of the embodiments shown in the drawings. On the contrary, the embodiments and modifications of the embodiments shall also be covered by the claimed protection. Furthermore, certain amendments, additions and other modifications will be obvious to a person of ordinary skill in the art familiar with the invention. Obvious modifications of the method disclosed herein and its embodiments as well as amendments, additions and other modifications of the disclosed system and its embodiments as well as further obvious applications of the invention, which are obvious to a person of ordinary skill in the art, shall be regarded as normal current or future expert knowledge of a person skilled in the relevant art and shall also be protected.

The features, advantages and details of the invention, which are disclosed in the drawings and claims, can be essential for the further development of the invention, either individually or in any combination. The basic concept of the invention shall not be limited to the exact forms or details of the embodiments shown below. It should also not be limited to a subject matter which would be restricted in comparison to the subjects described in the claims.

The elements followed by reference signs can indicate both a process and a device as well as the product of a process, if necessary at the same time.

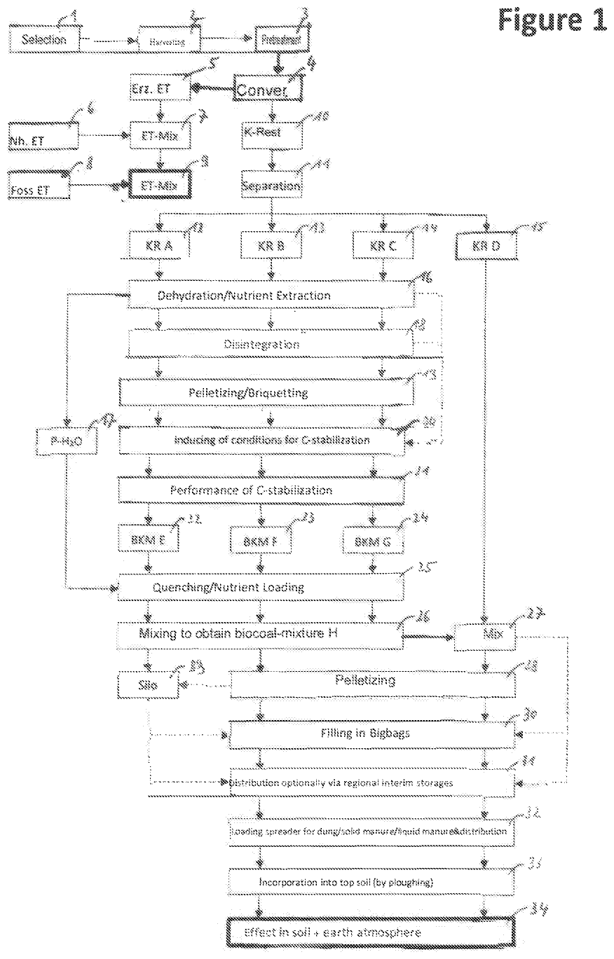

FIG. 1 shows a schematic diagram of a complex embodiment of the method and system according to the invention, which basically can also only consist of the conversion of biomass 4 by means of suitable devices into a generated energy carrier 5 as well as the generation of suitable conditions for the chemical-physical stabilization 20 of the atmospheric carbon still contained in the conversion residues and the conduction of carbon stabilization 21. These method steps are carried out in each case with suitable devices which are known to a person skilled in the art from the relevant prior art and which are described above, at least in part, and below. Even without sequestration 33 of the biochar or vegetable coal or biocoke produced, stabilization 21 of the atmospheric carbon already ensures that it can no longer react with atmospheric oxygen to form CO₂ or with hydrogen to form CH₄. This decarbonizes the atmosphere of the earth.

The at least one biomass to be used is selected in a method step 1 from a plurality of available biomasses (see claim 21). Some biomasses are only loaded with low GHG emission quantities up to the production stage of the biomass accumulation, some are even GHG-neutral (e.g. straw resulting from the grain harvest) and some are highly harmful to the environment, such as liquid manure or poultry manure stored outdoors, which emit CH₄ and N₂O. Their early use in a biomass conversion process prevents these GHG emissions, so that early utilization helps to avoid GHG emissions. This GHG avoidance is allocated to the product of the conversion process or is technically linked thereto, so that the utilization of certain farm manures can even lead to a GHG negative initial effect. The selection of one or more suitable biomasses for conversion process 4 can therefore be advantageous.

The selection 1 is also advantageous for a second reason. Some input materials are not suitable, or only suitable to a limited extent, for C-stabilization 20/21. Liquid input materials, such as fermented slurry, can only be carbonized using the HTC method. This method only provides partially stabilized biochar/vegetable coals, which do not remain stable for centuries or millennia. Therefore, a permanent sequestration with these HTC-coals is not possible (see chapter “Background”). Consequently, it is advantageous to select the input material 1 prior to the (first) conversion of the input material 1 into a generated energy carrier 5 in such a way that its conversion residue can still be used for chemical-physical stabilization that provides permanently stabilized carbon. This is the case with solid or lumpy lignocellulose-containing input materials, such as straw and wood, which are therefore preferably used in the method and system of FIG. 1. Sold or lumpy conversion residues containing straw and wood from a first biomass conversion can, for example, be subjected to pyrolysis, which provides permanently stabilized biochar/vegetable coals or biocoke. This makes a permanent decarbonization of the atmosphere of the earth possible.

The at least one biomass, which is preferably lignocellulose-containing biomass, more preferably straw, is harvested or collected in a step 2. In this context, harvesting and collection involve the transport of the at least one input material from the point of production to the point of use of the at least one input material. In particular, devices, apparatuses, installations and equipment can be used that use GHG-emission-reduced, preferably GHG-neutral and more preferably GHG-emission-negative fuels, heating mediums and combustion materials. These include, for example, combine harvesters, corn harvesters, tractors, wheel loaders, manitous, trucks, semitrailer tractors and all similar harvesting and transport machines with CNG or LNG drive known from the relevant prior art. These machines can use the energy carrier 5, energy carrier 6, energy carrier mix 7 or energy carrier mix 9 produced according to the method and system of FIG. 1 as fuel, wherein these fuels are GHG-emission-reduced, preferably GHG-neutral and in particular GHG-negative. By using such equipment with such drives and fuels, the GHG footprints and GHG emission values of the generated energy carriers 5, 7 and 9 are reduced, preferably to 0.0 gCO₂-eq/kWh_{Hi}, more preferably to less than 0.0 gCO₂-eq/kWh_{Hi}.

The process 2 can be limited to a collection if the at least one biomass is a by-product, residue or waste. Harvesting is required if the biomass is agricultural or forestry biomass or If it is an agricultural by-product (residue), such as straw. Straw, for example, must usually be pressed into straw bales in swaths after it has been deposited by the combine harvester since loose straw is not suitable for transport. The baling is carried out with tractor-drawn and tractor-driven straw bale presses, which usually achieve a pressing capacity of about 35 tons per hour. Alternatively, the straw deposited in the swath can also be pressed into pellets, highly suitable for transport, already in the field by tractor-drawn pellet presses (pellet harvesters). However, the pressing capacity of the pellet presses is currently only about 5 tons per hour, which blocks valuable tractor and personnel capacity, which is scarce especially during the harvesting campaign. With a bulk density of 600-700 kg/m³, straw pellets are much more suitable for transport than straw bales, in particular when using the long-distance means of transport rail or ship.

When using the truck as a means of transport, there is no further increase in transport efficiency at the moment when the load is using up the transport capacity of the truck. This is the case when the straw is compressed into high-pressure bales with a density of about 200 kg/m₃ and the load volume is exhausted.

In addition to pressing, the straw harvest also includes collecting the straw bales and loading the first means of transport. With collection trucks that are clamped to the straw baling press, a pre-collection can be carried out by accumulating 3-4 straw bales each before they are placed in a group on the stubble field. By placing the bales in groups, the distances travelled or the loading cycles of the loading equipment that load the (first) means of transport used to move the straw bales from the field are reduced.

In the case of smaller quantities of straw to be harvested, the (first) means of transport usually consists of agricultural trailers available on the farm, in the case of larger farms of low-loader semi-trailers which are mounted on tractor-drawn double-axle mountings or tractor-drawn low-floor trailers. The loading of the means of transport is usually done with front loaders (tractors or so-called manitous), which pick up the bales individually or in pairs and load them onto the means of transport. The loaded straw is transported from the first means of transport to the edge of the field or to the farm where it is unloaded again with front loaders or manitous and piled up outside to straw haystacks or stored in weather-protected warehouses. The route rarely exceeds 10 km. The straw bales can be cuboid or round. Bale collecting trucks are also known from Spain, with which square bales are collected and unloaded at the edge of the field.

For larger quantities of straw, the (second) means of transport consists of truck-towed trailers or semi-trailers towed by tractors. This means that the semitrailers drive directly onto the field or—if harmful compaction of the soil is to be avoided—at least to the edge of the field. Loading is usually carried out with wheel loaders which have a special grab with which up to 6 square bales can be grabbed at once and loaded onto the (second) means of transport. When so-called high-presses or ultra-high-presses are used, the density of the square bales reaches up to 0.210 t/m³, which almost fully utilizes the weight loading capacity of the trucks (about 20 t) when the loading volume is fully utilized. The straw can then be transported over several hundred kilometers, as is already the case today, namely from the Magdeburg area to Holland and Belgium, where there is a high demand for straw, for example for mushroom cultivation. Usually, however, the straw is temporarily stored at a regional straw storage facility, protected from arson, before it is transported over long distances. It is advantageous to use in the method and system shown in FIG. 1 the harvesting and transport technology that is used for larger quantities of straw because it is more energy-efficient than the small-sized technology usually used. Thus, harvesting can be carried out with less use of the valuable energy carriers 5, 7 or 9.

The (first) single-stage or multi-stage biomass conversion 4 can be any type of biomass conversion previously known from the relevant prior art that has the purpose of producing a marketable energy carrier. These include in particular the KIT Bioliq® process, the CLARIANT AG (formerly Süd-Chemie GmbH) Sunliquid® process, the IOGEN process for producing ethanol from lignocellulose-containing biomass, the CHOREN process, the Verbio AG process for producing biogas from straw, the Lehmann process for producing biogas from straw, the Fraunhofer process for producing biogas from biomass, the Jensen & Jensen process for producing biogas from straw, all Hoffmann processes for producing biogas from biomass, all processes by Lutz (Bekon) for producing biogas from biomass, all processes by Schiedermeier (BioFerm) for producing biogas from biomass, all processes by Eggersmann for producing biogas from biomass, all processes for producing ethanol from biomass, all processes for producing FT-fuel from biomass, all processes for producing methanol from biomass, all processes for producing DME from biomass, all other processes for producing fuel from biomass (cf. claim 1).

The different forms of single-stage or multi-stage biomass conversion 4 are carried out using devices, installations, apparatuses or systems which are previously known from the relevant prior art and which are suitable for this purpose, preferably in order to achieve economies of scale with installations of an industrial scale. More preferably, the devices for the single-stage or multi-stage conversion of biomass can consist of a selection of the following devices: Devices for the anaerobic bacterial fermentation of biomass into biogas and/or bio-methane, devices for the alcoholic fermentation of biomass into bioethanol or ligno-ethanol, devices for the carbonization of biomass into carbonization gas (weak gas), devices for the gasification of biomass into pyrolysis gas and/or pyrolysis slurry, devices for the transesterification of vegetable oils into bio-diesel (FAME), Devices for the hydration of vegetable oils in HVO (mineral oil refineries), devices for refining vegetable oils in HVO (NesteOil process), devices for the gasification/pyrolysis of biomass to process gas, devices for the conversion of biomass-derived process gas into synthesis gas, devices for the synthesis of biomass-derived synthesis gas into a Fischer-Tropsch fuel (FT-diesel, FT-gasoline, FT-kerosene, FT-methanol and the like), devices for the synthesis of methanol from biomass-derived gases, devices for DME synthesis, any combination of these devices (cf. claim 28). Single-stage or multi-stage biomass conversion 4 with biogas installations is particularly preferred, especially with agricultural biogas installations and at best with medium-sized or industrial biogas installations (cf. claim 34). When using these devices, the wet fermentation process in wet fermenters can be used, but also the solid fermentation process in sold fermenters, preferably in garage or plug flow fermenters. If garage fermenters are used, they are operated with a fermentation cycle of <180 days, preferably with a fermentation cycle of <60 days, more preferably with a fermentation cycle of <35 days, in particular with a fermentation cycle of <21 days and at best with a fermentation cycle of <14 days (cf. claim 34).

Before the (first) single-stage or multi-stage biomass conversion 4, the biomass, which is preferably lignocellulose-containing biomass, especially straw, can be pre-treated (but does not have to be). Conversion 4 is preferably an anaerobic bacterial fermentation carried out in a biogas installation, more preferably a solid fermentation and in particular a fermentation in a garage fermenter (cf. claim 23). Conversion 4 is carried out in devices which are previously known from the relevant prior art and which are suitable for such a conversion. Fermentation in a solid fermentation plant has the advantage that the fermentation residue 10 produced after fermentation is present in a heap (and not in liquid form) and thus after dehydration and/or drying in suitable devices a pyrolysis can be carried out which would only be possible with liquid conversion residues with a very high technical and energetic effort.

Depending on how long and intensively fermentation 4 is carried out in the at least one biogas installation (wet or solid fermentation installation) (Hydraulic Retention Time HRT), a more or less high conversion efficiency results. Long HRTs lead to desirable high conversion efficiencies and short HRTs to low conversion efficiencies. However, even with a very high conversion efficiency, a certain proportion of the atmospheric carbon contained in the input material always remains in the conversion residue, which is chemically and physically stabilized in suitable facilities according to the invention (cf. claims 1 and 27). In an advantageous embodiment of the invention shown in FIG. 1, the average hydraulic residence time of the fresh mass in the fermenter in the case of anaerobic (bacterial) fermentation is less than 250 days, preferably less than 120 days, more preferably less than 70 days, in particular less than 40 days and at best less than 20 days.

If straw or wood is used as an input material and pre-treatment 3 is carried out, pre-treatment 3 which is upstream of the conversion 4 can consist of a number of previously known measures which are previously known prior art as are the installations, apparatuses and systems used for this purpose. It is possible, for example, that the straw is subjected to comminution, preferably chopping or shredding, more preferably the comminution combination consisting of chopping or shredding and grinding, and in particular the comminution combination consisting of bale disintegration, chopping/shredding and grinding. In order to achieve a high conversion efficiency in the first conversion 4, it is advantageous to perform multi-stage comminution to an average final particle length of <20 cm, preferably to an average final particle length of <5 cm, more preferably to an average final particle length of <10 mm, in particular to a final particle length of <3 mm and in the best case to a final particle length of <1 mm (cf. claim 24). Pre-treatment of straw extrusion which crush and defiber the straw, and similar processes previously known from the prior art are also possible.

Before, during or after the method step of the single-stage or multi-stage conversion 4 of the selected biomass 1 into another marketable energy carrier 5, the biomass 1, which is preferably lignocellulose-containing biomass, more preferably straw and/or wood, can be subjected, along with or instead of the above described treatment, to a treatment consisting of an appropriate treatment previously known from the relevant prior art, preferably a selection from the following treatment methods: comminution, soaking/mixing/mashing in cold water or aqueous suspensions including lyes and acids, soaking/mixing/mashing in 20° C.-100° C. warm water or aqueous suspensions including lyes and acids, biological treatment with fungi, pressurization to >1 bar to 500 bar, treatment with >100° C. hot water, treatment with saturated steam, treatment by thermal pressure hydrolysis, treatment by wet oxidation, treatment by extrusion, ultrasonic treatment, treatment by steam reforming, treatment by steam explosion, drying, treatment with process water of any kind, treatment with process heat, treatment with enzymes, combination of a selection of these treatment methods by means of suitable installations, apparatuses or systems previously known from the relevant prior art. The schematic embodiment of FIG. 1 only shows the pre-treatment 3 of input materials ½, although it is understood that the above measures can also be applied to conversion residues 10 and/or to all intermediate products produced during a multi-stage conversion 4. Straw and/or wood, for example, can be subjected to a multi-stage conversion 4 with or without pre-treatment 3, namely fermented in a first fermentation, wherein the fermentation residues from this fermentation are treated according to the above-mentioned treatment methods and the fermentation residues treated in this way are fermented again in at least a second fermentation (cf. claim 25).

Accordingly, the system shown in FIG. 1 can include devices capable of subjecting biomass ½ to a selection of the following treatment methods before or during conversion 4 or residues 10 after biomass conversion 4: soaking/mixing/mashing in cold water or aqueous suspensions, soaking/mixing/mashing in 20° C.-100° C. warm water or aqueous suspensions, biological treatment with fungi, pressurization to >1 bar-500 bar, treatment with >100° C. hot water, treatment with saturated steam, treatment by thermal pressure hydrolysis, steam explosion, wet oxidation treatment, heating, extrusion treatment, ultrasonic treatment, steam reforming treatment, evaporation, sedimentation, crystallization, catalysis, drying, use of polymers, phase separation, particle extraction, combination of a selection of these treatment methods (cf. claim 39).

Before, during or after the method step of the single-stage or multi-stage conversion 4 of the selected biomass 1 into another marketable energy carrier 5, the biomass 1, which is preferably lignocellulose-containing biomass, more preferably straw, can be provided with suitable additives previously known from the relevant prior art, preferably with a selection from the following additives: Lime, enzymes, enzyme-containing solutions, fungi, acids, lyes, yeasts, water, recycled process water, purified process water, filtered process water, ultrafiltrated process water, process water subjected to reverse osmosis, treated process water, acid-water mixtures, lye-water mixtures, percolate, silage seepage juices, slurry, micro-organisms, any cereal grain stillage from ethanol production, any residue from the production of ligno-ethanol, any by-product/residue from the production of pyrolysis or synthesis gas, any by-product/residue from FT synthesis, any by-product/residue from DME synthesis, any by-product/residue from methanol synthesis, any sugar beet stillage from ethanol production, combination of two or more of these additives (cf. claim 23). The admixture of the additives is preferably carried out with suitable apparatuses, installations or systems previously known from the relevant prior art.

At least a portion of the residues from the single-stage or multi-stage biomass conversion 4 (conversion residue 10) is recuperated and made available to the further method steps (cf. claim 22). The conversion residues 10 can in particular be residues from a single-stage or multi-stage anaerobic bacterial fermentation 4 (fermentation residues).

The energy carrier 5 produced in the process step of single-stage or multi-stage conversion 4 can be a selection of biogas, bio-methane, pyrolysis gas, synthesis gas, bio-diesel, bio-kerosene, Fischer-Tropsch fuel, bio-methanol, DME or bio-ethanol (cf. claim 14).

Preferably, this energy carrier 5 is processed in such a way that it can be used as fuel, heating medium or combustion material, preferably as a transport fuel, more preferable as a road fuel (cf. claim 14). Conversion 4 is preferably carried out with suitable apparatuses, installations or systems previously known from the relevant prior art. The energy carrier 5 produced can assume a gaseous or liquid aggregate state, i.e. for the liquid aggregate state the energy carrier 5 is liquefied with suitable devices previously known from the relevant prior art.

In an advantageous embodiment of the invention shown in FIG. 1, after the production, distribution and use of the energy carrier 5 produced, which is preferably a fuel, more preferable a gas fuel and in particular bio-methane, there is a lower amount of greenhouse gas in the atmosphere of the earth than after the production, distribution and use of an equal amount of energy of the compatible fossil counterpart of the energy carrier 5 produced, wherein mineral diesel fuel is the compatible fossil counterpart for all diesel substitutes, mineral fuel for Otto engines (gasoline) the compatible fossil equivalent for all substitutes of fuel for Otto engines, mineral kerosene the compatible fossil equivalent for all kerosene substitutes, natural gas (CNG) the compatible fossil equivalent for all natural gas substitutes, LNG the compatible fossil equivalent for all LNG substitutes, LPG the compatible fossil equivalent for all LPG substitutes and the weighted average of mineral fuel for Otto engines and mineral diesel the compatible fossil equivalent for all other fuels, heating mediums and combustion materials (cf. claim 15).

In another advantageous embodiment, after the production, distribution and use of the generated energy carrier 5, which is preferably a fuel, more preferably a gas fuel and in particular bio-methane, there is a smaller amount of greenhouse gas in the atmosphere of the earth than before, i.e. the generated energy carrier 5 is GHG-negative (cf. claim 16).

Preferably, the generated energy carrier 5 is mixed with a compatible sustainable energy carrier 6 to form an energy carrier mix 7, wherein the compatible and sustainably generated energy carrier 6 can come from another conversion process which can have a higher GHG emission value than energy carrier 5. The energy source mix 7 resulting from the mixing is used more preferably as a fuel in traffic. The proportion of energy carrier 5 in the energy carrier mix 7 can be between 0% and 100%, and accordingly the proportion of sustainable energy carrier 6 in the energy carrier mix 7 can be between 100% and 0%. The mixing is preferably carried out with suitable apparatuses, installations or systems previously known from the relevant prior art.

The mixing of the produced energy carrier 5 with the compatible, sustainably produced energy carrier 6 to form an energy carrier mix 7 is preferably carried out in such a way that after the production, distribution and use of the produced energy carrier mix 7 there is a lower amount of greenhouse gas in the atmosphere of the earth according to life cycle analysis or stoichiometric analysis than after the production, distribution and use of an equal amount of energy of the fossil counterpart of the produced energy carrier mix 7, wherein mineral diesel fuel is the fossil equivalent for all diesel substitutes, mineral fuel for Otto engines (gasoline) is the fossil equivalent for all substitutes for Otto engines, mineral kerosene is the fossil equivalent for all kerosene substitutes, natural gas (CNG) is the fossil equivalent for all natural gas substitutes, LNG the fossil equivalent for all LNG substitutes, LPG the fossil equivalent for all LPG substitutes and the weighted average from mineral fuel for Otto engines and mineral diesel the fossil equivalent for all other fuels, heating mediums and combustion materials (cf. claim 17).

In an advantageous embodiment, the produced energy carrier 5 is mixed with the compatible, sustainably produced energy carrier 6 in such a way that after the production, distribution and use of the produced energy carrier mix 7 according to life cycle analysis or stoichiometric analysis, a smaller amount of greenhouse gas is present in the atmosphere of the earth than before. This means that the GHG emission value of the energy source mix 7 is GHG negative (cf. claim 18).

The energy carrier 5 is preferably biogas processed into bio-methane, more preferably straw-gas, which was produced by anaerobic bacterial fermentation at least proportionally from straw-containing and possibly other input materials (farm manure, beet and potato haulms as well as legume waste, wood). More preferably, the compatible sustainable energy carrier 6 is syn-methane, which was produced from wind power and atmospheric CO₂, or is another biogas. In particular, energy carrier 5 and energy carrier 6 are mixed in such a way that after the production, distribution and use of the energy carrier mix according to life cycle analysis or stoichiometric analysis, a lower (measured in tons of CO₂ equivalent) amount of greenhouse gas is present in the atmosphere of the earth than after the production, distribution and use of an equal amount of energy of the fossil equivalent of the energy source mix 7, at best in such a way that after the production, distribution and use of the energy carrier mix 7 there is a smaller amount of greenhouse gas in the atmosphere of the earth than before, i.e. the energy carrier mix 7 produced is GHG negative. In the case of straw-gas, the energy carrier 5 can be mixed with syn-methane 6 in such a way that the resulting mixed gas 7 is GHG neutral (cf. claim 19). The quantity of added syn-methane 6 depends on its GHG value because the higher the GHG value, the lower the syn-ethane 6 quantity that can be admixed. The mixing is preferably carried out with suitable apparatuses, installations or systems previously known from the relevant prior art. The energy source mix 7 can assume a gaseous or liquid aggregate state, i.e. for the liquid aggregate state, the energy carrier 5 produced and the sustainably produced compatible energy carrier 6 are liquefied before or after mixing with suitable devices known from the relevant prior art.

Preferably, the energy carrier mix 7 and at least one compatible fossil energy carrier 8 can be mixed as the end product to produce an energy carrier mix 9. The energy carrier mix 9 resulting from the mixing is used more preferably as a fuel in traffic. The proportion of energy carrier 7 in the end product 9 can be between 0.1% and 100.0%, and accordingly the proportion of fossil energy carrier 8 in the end product 9 can be between 99.9% and 0.0%. In particular, the energy carrier mix 7 is biogas processed to bio-methane mixed with other biogas or syn-methane 6, at best straw-gas produced from straw-containing input materials mixed with other biogas or syn-methane. The mixing is preferably carried out with suitable apparatuses, installations or systems previously known from the relevant prior art.

The compatible fossil energy carrier 8 is preferably natural gas or LNG (liquefied natural gas). The mixing of the energy carrier mix 7 and the fossil energy carrier 8 is more preferred in such a way that, according to life cycle analysis or stoichiometric analysis, a lower greenhouse gas amount (measured in tons of CO₂ equivalent) is present in the atmosphere of the earth after the production, distribution and use of the energy carrier mix 9 than after the production, distribution and use of an equal energy amount of the fossil equivalent of the energy carrier mixture 9, more preferably in such a way that after the production, distribution and use of the energy carrier mixture 9 there is a smaller amount of greenhouse gas in the atmosphere of the earth than before, i.e. the generated energy carrier mixture 9 is GHG negative.

In the case of straw-gas, the energy carrier mix 7 can be mixed with natural gas (CNG) 6 in such a way that the resulting mixed gas is GHG neutral. The quantity of the added natural gas 6 here depends on the GHG emission value of the energy carrier mix 7, because the higher the GHG emission value, the lower the quantity of natural gas 8 that can be admixed.

The energy carrier mix 9 can assume a gaseous or liquid aggregate state, i.e. for the liquid aggregate state the energy carrier mix 7 produced and the compatible fossil energy carrier 6 are liquefied before or after mixing with suitable devices known from the relevant prior art.

The conversion residue 10 resulting from the single-stage or multi-stage biomass conversion 4, which is preferably an anaerobic bacterial fermentation, is at least partially recuperated (cf. claim 22). The amount of the resulting conversion residue 10 depends on the conversion efficiency achieved by conversion 4 of the biomass (reference numerals 1 and 2) into the energy carrier 5. If the resulting conversion residues are completely recuperated and the conversion efficiency is e.g. 10%, about 90% of the atmospheric carbon contained in the biogenic input material is still contained in conversion residue 10. If the conversion efficiency with complete recuperation of the conversion residue is e.g. 70%, about 90% of the atmospheric carbon contained in the biogenic input material is still contained in conversion residue 10.

In the process and system variant shown in FIG. 1, the recuperated proportion of conversion residue stream leaving the method step of single-stage or multi-stage biomass conversion is divided in the method and system variant shown in FIG. 1 in an additional method step 11 into up to four partial streams A to D (reference signs 12 to 15), namely into the first partial stream “production of stabilized pyrolysis coal”, into the second partial stream “production of partly stabilized torrefaction or HTC coal”, into the third partial stream “production of unstabilized biochar/vegetable coal/biocoke” and into the fourth partial stream “non-carbonized conversion residues” (cf. claim 9). The purpose of this division is to obtain a biochar/vegetable coal mixture or a biocoke mixture that is better suited to forest and agricultural soils than biochar/vegetable coal that consists only of fully stabilized, partially stabilized or unstabilized atmospheric carbon—although these options shall not be excluded. In order to avoid repetitions, reference is made in this respect to the above descriptions and explanations.

The partial streams A to D (RS 12 to 15) can each have a share of 0% to 100% of the total stream, i.e. each partial stream 12 to 15 can represent the total stream as well as zero and each share in between (cf. claim 9). The partial stream A with reference sign 12 “production of stabilized pyrolysis coal” preferably has a share of >1% of the total stream, more preferably a share of >25%, in particular a share of >50% and in the best case a share of >75% (cf. claim 9). The higher the proportion of fully stabilized carbon in the biochar/vegetable coal mixture, the greater the permanent decarbonization effect and the resulting effect on the GHG emission values of the energy carriers 5, 7 and 9. The partial streams A to D can be divided using a switch that divides the conversion residue stream 10 leaving conversion 4 into various suitable conveyors, such as lines, conveyor belts, chutes, elevators, etc. (not shown) or containers.

In the method steps following stream distribution 11, the respective partial stream is carried out by means of suitable installations, apparatuses or systems, which is indicated by the partial stream designation (cf. claim 9). This means that the partial stream A (RS 12) serves for the full stabilization of atmospheric carbon, wherein this full stabilization is carried out in method steps 20 and 21, preferably by means of pyrolysis, more preferably by means of high-temperature pyrolysis and in particular by means of high-temperature pyrolysis, which is carried out after a slowly carried out heating. Biochar/vegetable coal with fully stabilized atmospheric carbon advantageously increases the permanent humus content of the soil.

Accordingly, partial stream B (reference sign 13) serves for partial stabilization of atmospheric carbon in method steps 20 and 21, preferably by means of HTC, more preferably by means of low-temperature pyrolysis (torrefication) and in particular by means of low-temperature pyrolysis (torrefication) which is carried out after slow heating. Biochar/vegetable coal with partially stabilized atmospheric carbon increases the nutrient humus content of the soil in an advantageous way.

Partial stream C (reference numeral 14) is used to produce biochar/vegetable coal containing unstabilized atmospheric carbon. For this purpose, inter alia any carbonization process can be used, including pyrolysis, which must be carried out only briefly and/or aggressively enough (very fast heating, very high reaction temperatures). Biochar/vegetable coal with unstabilized atmospheric carbon increases the OPS or OSS content of the soil in an advantageous way.

The partial stream D (RS 15) serves to advantageously increase the OPS or OSS content of the soil. It is comparable with conversion residues 10, which are applied directly and without after-treatment as fertilizer on agricultural land after a single-stage or multi-stage conversion 4. Such conversion residues are e.g. fermentation residues from anaerobic bacterial fermentation or stillage from the alcoholic fermentation of biomass to bio-ethanol.

The up to four products (biochar masses E to G and conversion residue D), which may or may not be produced in the method steps following conversion residue division 11, can generally be processed or treated in parallel or in series with the respective installations and apparatuses. In the case of serial processing, the unprocessed partial streams are conveyed to suitable intermediate storage facilities (not shown) by means of suitable conveyor technology (not shown) which is previously known from the relevant prior art, and stored until further processing is started. It is thus possible to first carry out high-temperature pyrolysis with one and the same device for C-stabilization and then torrefaction with a significantly lower reaction temperature and/or a significantly shorter reaction time. Devices which are suitable for high reaction temperatures are usually also suitable for lower reaction temperatures. The same applies to the parameters heating rate, reaction time and reaction pressure.

After dividing the residual conversion stream 10 into the partial streams A to D (reference signs 12 to 15), the partial streams A to C pass through three optional method steps, namely dehydration/nutrient extraction 16, disintegration 18 and pelletization/briquetting 19. However, such treatment is not absolutely necessary; the objective is also achieved if the conversion residues 10 or partial streams 12 to 14 do not pass through method steps 16 to 19 and are led straight to method steps 20 and 21. The division is preferably carried out with a suitable switch or the like which is previously known from the relevant prior art.

In an advantageous embodiment (not shown in FIG. 1) of the method and system shown in FIG. 1, the residual conversion partial streams A to D (RS 12 to 15) generated by means of division 11 can be stored in a silo, container, bunker or similar devices previously known from the relevant prior art until there is a need for them. This demand can be based on downstream dehydration/nutrient extraction 16, downstream desintegration 18, downstream pelleting/briquetting 19, downstream C-stabilization 20/21 or downstream mixing 27.

In the method step of dehydration/nutrient extraction 16, the conversion residues 12 to 14 are dewatered by means of suitable installations, apparatuses or systems previously known from the relevant prior art. Preferably, devices are included which are capable of extracting or separating at least a portion of the organic nutrients still contained in the residues of the single-stage or multi-stage biomass conversion or at least a portion of the water still contained in the residues of the single-stage or multi-stage biomass conversion, which devices can more preferably consist of a selection from the following devices: soaking, mixing, mashing and similar devices, spinners, centrifuges, cyclones, decanters, presses, separators, screens, filtration devices, ultrafiltration devices, reverse osmosis devices, similar devices, combination of these devices (cf. claim 31).

Dewatering 16 is advantageous because the downstream stabilization of the atmospheric carbon functions the better and more effective, the higher the dry substance content of the biomass to be treated in method steps 20 and 21. In an advantageous embodiment of the method shown in FIG. 1, dewatering is therefore carried out in such a way that the dry substance content (DS content) of the resulting, more solid phase is >35%, preferably >50% DS, more preferably >60% DS (cf. claim 4). For this purpose, devices previously known from the relevant prior art are used, preferably those which are suitable for extracting or separating at least a portion of the water contained in the conversion residues 12 to 14, wherein these devices more preferably consist of a selection from the following devices: Spinners, centrifuges, cyclones, decanters, presses, separators, screens, filtration devices, ultrafiltration devices, reverse osmosis devices, similar devices, combinations of these devices. These dewatering devices are preferably suitable for dewatering the conversion residues 12 to 14 from the single-stage or multi-stage biomass conversion 4, preferably to a DS content of >35% DS, more preferably to a DS content of >50% DS and in particular to >60% DS (cf. claim 31).

In addition to the more solid phase, the product of dewatering 16 is a more liquid phase, which is referred to here as process water 17. “More solid” in this context means that the solid phase also contains water just as the “more liquid” phase contains dry substance. This means that the DS content of the more solid phase is not 100% but—unless otherwise stated—only higher than the DS content of the more liquid phase.

The process water 17 can be used to quench, if necessary, the biochar/vegetable coal coming from the C-stabilization or the biocoke coming from the C-stabilization, which is usually very hot. This is done in method step 25. However, it can also be used to replace (fresh) water requirements elsewhere in the method, e.g. In method step 3 (e.g. pretreatment by soaking in water or aqueous suspensions) and/or in method step 4 (e.g. anaerobic bacterial fermentation in a wet fermenter) and/or in method step 19 (evaporation of conversion residues 12 to 14 to be pelletized or briquetted).

The system shown in FIG. 1 can comprise devices which are previously known from the relevant prior art and are suitable for recuperating process water produced in the method and supplying it to other system components (lines, containers, bunkers, reservoirs, pumps, etc.), preferably after processing or purification of the recuperated process water in corresponding devices previously known from the relevant prior art, more preferably after processing or purification of the recuperated process water by means of a selection from the following devices: spinners, centrifuges, cyclones, decanters, presses, separators, screens, filtration devices, ultrafiltration devices, reverse osmosis devices, similar devices, combinations of these devices (cf. claims 31 and 32).

The process water 17 usually contains valuable organic nutrients to a technically relevant extent. After the (first) single-stage or multi-stage biomass conversion 4, which is preferably an anaerobic fermentation, the (organic) nutrients contained in the biogenic input material are still largely present, i.e. they are contained in the recuperated conversion residue 10 or in the conversion residue partial streams 12 to 15. Most of them are in solution. This means that the process water 17 leaving the conversion residues 12 to 14 can be aqueous suspensions enriched with (organic) nutrients. The composition of these nutrients corresponds exactly to that of the composition of the nutrients removed from the topsoil during biomass cultivation or biomass growth. As shown above, it is advantageous for the field topsoil if the applied biochar/vegetable coal is loaded with nutrients. To avoid repetitions, reference is made to the relevant sections of the above Illustrations. Since the most of the nutrients contained in the conversion residues 12 to 14 are destroyed and/or lost during chemical-physical stabilization 20/21 of the atmospheric carbon still contained in the conversion residues 12 to 14, it is advantageous to extract at least part of these nutrients from the conversion residues prior to C-stabilization by means of suitable installations, apparatuses or systems previously known from the relevant prior art and to then use them to load the biochar/vegetable coal 22 to 24 produced (cf. claim 31). The biochar/vegetable coals are loaded with nutrients in method step 25 (see below).

According to the invention, the organic nutrients still contained in the residues of the single-stage or multi-stage biomass conversion 4 are therefore at least partially removed, prior to the chemical-physical treatment of the conversion residues 12 to 14, from these conversion residues by means of suitable apparatuses, installations or systems previously known from the relevant prior art, preferably together with process water 17, more preferably by a selection from the methods of spinning, decanting, pressing, separation, filtration, reverse osmosis, addition of polymers, combination of these method steps and in particular with corresponding suitable apparatuses and installations (cf. claim 4).

According to the invention, the process water 17 obtained, which is preferably an aqueous suspension that contains organic nutrients, is returned to the process, more preferably by means of suitable apparatuses, such as lines, containers, tanks, bunkers and pumps (cf. claim 4) and in particular after purification of inhibitors or residual materials.

The conversion residues 12 to 14, which may have been dehydrated in method step 16 and/or from which organic nutrients may have been extracted in the same method step 16, can be disintegrated in an optional method step 18 using suitable disintegration apparatuses previously known from the relevant prior art (cf. claim 38), namely to an average particle length of 0.01 mm to 300 mm, preferably to an average particle length of 0.1 mm to 100 mm, more preferably to average particle lengths of 1.0 mm to 30 mm and in particular to average particle lengths of 1.5 mm to 20 mm. Disintegration can be advantageous because the biochar/vegetable coals 22-24 produced can be better distributed on (arable) soil and/or better incorporated into (arable) soil. A high degree of fineness is particularly advantageous if the biochar/vegetable coals 22-24 produced shall be mixed with slurry, with or without nutrient loading, in order to reduce or prevent unpleasant odors from the slurry and/or to reduce environmentally harmful N₂O emissions resulting from the spreading of the slurry. For this purpose of easier distribution by means of a slurry distributor, disintegration 18 can also be carried out later in the process, for example immediately after C-stabilization 20/21, immediately after quenching/loading with nutrients 25, immediately after mixing to a biochar mixture H 26 or immediately after mixing to a biochar conversion residue mixture 27.

The optional disintegration 18 with suitable apparatuses previously known from the relevant prior art can be advantageous for a second reason, namely as a preparatory measure for a possibly downstream pelletization or briquetting 19. If the chemical-physical stabilization 20/21 is to consist of carbonization of the conversion residues 12 to 14 and these consist of pyrolysis, pyrolysis methods and apparatuses can be used which can only pyrolyze input materials that are lumpy or cut into lumps. In order to be able to use these methods and apparatuses, pelletization/briquetting of the conversion residues 12 to 14 is required. This in turn can require comminution of the conversion residues 12 to 14. If the comminution has not already taken place during the pretreatment 3 to the degree of fineness required for pelleting/briquetting, the comminution must then take place at the latest before method step 19 (pelleting/briquetting).

The optional disintegration 18 with suitable apparatuses previously known from the relevant prior art can be advantageous for a third reason, namely as a preparatory measure for the downstream C-stabilization 20/21. If the chemical-physical stabilization 20/21 is to consist of a carbonization of the conversion residues 12 to 14 and the latter of a pyrolysis, pyrolysis processes and apparatuses can be used which can only pyrolyze input materials with a certain degree of fineness. The PYREG 500 pyrolysis system from Pyreg GmbH currently only processes input materials that do not exceed a certain particle length.

The optional pelletizing/briquetting 19 can be necessary if the chemical-physical stabilization 20/21 shall consist of a carbonization of the conversion residues 12 to 14 and the latter of a pyrolysis. In this case, pyrolysis processes and apparatuses can be used which can only pyrolyze materials that are lumpy or cut into lumps. In order to be able to use these methods and apparatuses, pelletizing/briquetting 19 of the conversion residues 12 to 14 can be necessary.

Accordingly, the system of FIG. 1 can include devices which are previously known from the prior art and can pelletize or briquette the conversion residues prior to C-stabilization 21 and/or perform the associated sub-functions, namely to vaporize or dry the mass to be pelleted/briquetted, if necessary, and to cool, store and transport the pellets/briquettes produced (cf. claim 33).

The generation of conditions allowing at least partial chemical-physical stabilization of the atmospheric carbon still present in the residues of the (first) single-stage or multi-stage biomass conversion (digestion, fermentation, pyrolysis or synthesis residues) (RS 20) can refer to any methods and apparatuses previously known from the relevant prior art for the chemical-physical carbon stabilization. In the case of carbonization of the (possibly pre-treated or post-treated) conversion residues 12 to 14, this method step 20 can include the selection of the carbonization process, the selection of the aggregate state of the mass to be carbonized, the selection of the reaction vessel (reactor), the heating to a certain reaction temperature, the heating to reaction temperature in a certain period, the conduction of the reaction for a certain (period of) time, pressurization of the reaction mass, a certain kind of pre-treatment of the reaction mass, the kind of cooling the reaction product, another way of post-treatment of the reaction product and the selection of the appropriate devices. The execution of these parameters depends on the partial conversion stream 12 to 14 or the type of biocoal mass E to G (RS 22 to 24) to be used, as described below in the comment on step 21.

In a preferred embodiment of the method shown in FIG. 1, the conditions of method step 20 and/or the implementation of method step 21 are selected or set in such a way that the atmospheric carbon still contained in the conversion residues 12 and 14 is at least partially stabilized such that it is degraded (mineralized) within a certain period of time by less than 30%, preferably less than 20%, in particular less than 10% and in the best case less than 5% by the processes of soil respiration, weathering, aerobic rotting and/or reaction with atmospheric oxygen, wherein the certain period of time can be a selection from the following periods of time: 10 years, 30 years, 100 years, 500 years, 1.000 years, 10,000 years, 100,000 years, >100,000 years (cf. claim 2).

Accordingly, the system shown in FIG. 1 comprises devices which are previously known from the relevant prior art and can carbonize the conversion residues 12 and 13 in such a way that the carbon content of the resulting biochar masses 22 and 23 is degraded (mineralized) within a given period of time by less than 50%, preferably less than 20%, more preferably less than 10% and in particular less than 5%, by the processes of soil respiration, weathering, aerobic rotting and/or reaction with atmospheric oxygen, wherein the given period of time can be a selection from the following periods of time: 10 years, 30 years, 100 years, 500 years, 1.000 years, 10,000 years, 100,000 years, >100,000 years (cf. claim 30).

This degradation resistance is achieved in the case of carbonization of the conversion residues 12 and 13 when the molar H/C ratio of the produced biochar/vegetable coal/biocoke 22 and 23 is <0.8, preferably <0.6, or when its molar O/C ratio is <0.8, preferably <0.4. In particular, this degradation resistance is achieved when, in the case of carbonization, the molar H/C ratio of the biochar/vegetable coal/biocoke 22 and 23 produced is <0.8, preferably <0.6, and when its molar O/C ratio is <0.8, preferably <0.4. In an advantageous embodiment of the method and system described in FIG. 1, the conditions of method step 20 are therefore selected or set such that the molar H/C ratio of the biochar/vegetable coal/biocoke 22 and 23 produced is <0.8, preferably <0.6, and/or its molar O/C ratio is <0.8, preferably <0.4 (cf. claim 6).

These basic results can be achieved by selecting a particular carbonization process and/or keeping the reaction temperature relatively high and/or by heating to reaction temperature for a relatively long time, i.e. relatively slowly. In an advantageous embodiment of the method and system of FIG. 1, the conditions for the chemical-physical treatment of the conversion residues (RS 20) are therefore preferably set in such a way that the thermal or thermo-chemical carbonization of the conversion residues 12 and 13 (possibly treated according to 16, 18 or 19) to form biochar/vegetable coal/bio-coke is carried out by a selection from the following thermo-chemical carbonization processes: Pyrolysis, carbonization, torrefaction, hydrothermal carbonization (HTC), vapothermal carbonization, gasification and any combination of these treatment methods. This carbonization is preferably carried out by pyrolysis or torrefaction.

Accordingly, the system shown in FIG. 1 comprises devices previously known from the relevant prior art for the carbonization of conversion residues, preferably those suitable for pyrolysis and/or torrefaction (cf. claim 36).

The fundamental results listed above can also be achieved by the physico-chemical stabilization of atmospheric carbon by carbonization of the conversion residues 12 to 14 under oxygen deficiency at reaction temperatures of 100° C.-1600° C., preferably at reaction temperatures of 200° C.-1,200° C., more preferably at reaction temperatures of 300° C.-1,000° C., in particular at reaction temperatures of 350° C.-1,000° C. and in the best case at reaction temperatures of 400° C.-900° C. (cf. claim 20). Accordingly, the system of FIG. 1 comprises suitable devices previously known from the relevant prior art for the carbonization of conversion residues, preferably those suitable for ensuring reaction temperatures of 100° C.-1600° C., preferably reaction temperatures of 200° C.-1,200° C., more preferably reaction temperatures of 300° C.-1,000° C., in particular reaction temperatures of 350° C.-1,000° C. and in the best case reaction temperatures of 400° C.-900° C. (cf. claim 36).

The conversion residues A (RS 12) are preferably subjected to pyrolysis, more preferably to high-temperature pyrolysis, wherein the proportion of the conversion residues A in the total recuperated conversion residue stream 10 preferably has a proportion of >1%, more preferably >50% and in particular >75%.

The conversion residues B (RS 13) are preferably subjected to low-temperature pyrolysis, HTC or torrefaction, wherein the proportion of the conversion residues B in the total recuperated conversion residue stream 10 preferably has a proportion of <99%, more preferably <50%, in particular <25% and at best of <10%.

These basic results listed above can also be achieved by heating the conversion residue 12 to 14 to be treated to reaction temperature for longer than 1 second, preferably longer than 10 minutes, more preferably longer than 50 minutes and in particular longer than 100 minutes (cf. claim 20). The less aggressive conditions for C-stabilization 20 lead to better or more complete outgassing of conversion residues 12 to 14, leaving a firmer and less reactive carbon backbone behind, which in turn results in higher degradation resistance.

These fundamental results listed above can also be achieved in that the conversion residues 12 to 14 (possibly treated according to 16, 18 or 19) have the lowest possible residual water content. In an advantageous embodiment of the method shown in FIG. 1, dehydrated (dewatered) conversion residues 12 to 14 are therefore used in method steps 20/21 because carbonization by pyrolysis or torrefaction functions better in the sense of the objective of “permanent C-stabilization”, the lower the residual water content of the biomass treated in method step 21. Some pyrolysis installations, for example, require dewatering to at least 50% dry substance (DS). The conditions for C-stabilization 20 are preferably set so that the dewatering of the conversion residues 12 to 14 used for this purpose is carried out to >35% DS, more preferably >50% DS and in particular >60% DS (cf. claim 5).

In a preferred embodiment of the method shown in FIG. 1, the conditions of method step 20 are selected or set in such a way that the loss of atmospheric carbon which inevitably occurs during at least partial chemical-physical stabilization of the conversion residues is a maximum of 99%, preferably a maximum of 60%, more preferably a maximum of 40% and in particular a maximum of 30%. This can be achieved inter alia by heating the conversion residues 12 to 14 to be treated to reaction temperature for longer than 1 second, preferably longer than 10 minutes, more preferably longer than 50 minutes and in particular longer than 100 minutes (see above; cf. claim 20). This can also be achieved by a relatively long reaction time, preferably longer than 1 second, more preferably longer than 60 minutes, in particular longer than 240 minutes and at best longer than 600 minutes.

The devices of generating conditions allowing at least partial chemical-physical stabilization of the atmospheric carbon still present in the biomass conversion residues can include any devices known from the relevant prior art capable of carrying out chemical-physical treatment of such residues. They preferably include devices for the thermal or thermo-chemical carbonization of conversion residues 12 to 14 to form biochar/vegetable coal/biocoke 22 to 24, more preferably they include a selection from the following devices for the thermo-chemical carbonization of biomass to form biochar/vegetable coal/biocoke: pyrolysis devices, carbonization devices, torrefaction devices, hydrothermal carbonization (HTC) devices, vapothermal carbonization devices, gasification devices, any combination of these devices, wherein the devices for carbonizing the conversion residues are preferably suitable for carrying out the carbonization under oxygen deficiency and/or at reaction temperatures of 100° C.-1600° C., more preferably at reaction temperatures of 200° C.-1.200° C., in particular at reaction temperatures of 300° C.-1,000° C., in an even better case at reaction temperatures of 350° C.-950° C. and in the best case at reaction temperatures of 400° C.-900° C. (cf. claim 29).

In an advantageous embodiment of the system shown in FIG. 1, the devices for the generation of conditions allowing at least partial chemical-physical stabilization of the atmospheric carbon still contained in the residues of the biomass conversion are operated in such a way that the carbon fraction of the produced biochar/vegetable coals or the biocoke produced is degraded (mineralized) within a given period of time by less than 50%, more preferably less than 20%, in particular less than 10% and in the best case less than 5% by the processes of soil respiration, weathering, aerobic rotting and/or reaction with atmospheric oxygen, wherein the given period of time can be a selection from the following periods of time: 10 years, 30 years, 100 years, 500 years, 1,000 years, 10,000 years, 100,000 years, >100,000 years.

Once the conditions for C-stabilization have been set as desired in method step 20, C-stabilization 21 is carried out. The way in which the atmospheric carbon contained in conversion residues 12 to 14 is stabilized can be part of the conditions set in method step 20, i.e. method steps 20 and 21 are closely linked. For example, the rate of heating of the conversion residues 12 to 14 can be both a parameter for creating conditions for C-stabilization 20 and a parameter for performing C-stabilization 21. If the heating of the conversion residues 12 to 14 to be treated to reaction temperature falls under method step 21, it also takes longer than 1 second, preferably longer than 10 minutes, more preferably longer than 50 minutes and in particular longer than 100 minutes. The reaction time parameter also plays a role in both method step 20 and method step 21. C-stabilization 21 is therefore preferably carried out over a period of time longer than 1 second, more preferably longer than 60 minutes, in particular longer than 240 minutes and at best longer than 600 minutes.

In an advantageous embodiment of the method and system shown in FIG. 1, C-stabilization 20/21 is carried out under pressure, preferably at a reaction pressure of >1 bar, more preferably >5 bar and in particular >10 bar.

The method according to the invention is the more efficient in terms of a GHG emission reduction of the generated energy carrier 5 the lower the loss of (atmospheric) carbon during the C-stabilization 21. The chemical-physical stabilization of the atmospheric carbon contained in the conversion residues 12 to 14 or the carbonization of the conversion residues 12 to 14 is therefore preferably carried out in such a way that the occurring loss of atmospheric carbon based on the state before C-stabilization conversion residue carbonization is a maximum of 99%, more preferably a maximum of 60%, in particular a maximum of 40% and at best a maximum of 30% (cf. claim 3). As stated above, this can be achieved by slow heating to reaction temperature and/or a high reaction temperature and/or a long reaction time.

During the application of the produced biochar/vegetable coal according to the invention or the produced biocoke in the soil, not only the (stabilized) carbon content in the biochar/vegetable coal has an effect but also the entire biochar/vegetable coal including the other substances contained in it that are not made of carbon. Accordingly, the consumers of the biochar/vegetable coals produced primarily consider the total quantity purchased from the producer or applied per hectare. It is therefore advantageous in terms of a maximum output of biochar/vegetable coal or biocoke if the loss of dry substance occurring during carbonization (RS 21) of the conversion residues 12 to 14 is a maximum of 99%, more preferably a maximum of 60%, in particular a maximum of 40% and at best a maximum of 30% (cf. claim 6).

The biochar/vegetable coals 22 to 24 or biocoke produced according to the method shown in FIG. 1 are all the more valuable the higher their carbon content is. In an advantageous embodiment of the example of the method according to the invention shown in FIG. 1, C-stabilization 21 is therefore carried out in such a way that the carbon content of the produced biochar/vegetable coal or the produced biocoke is at least 20%, preferably at least 40%, more preferably at least 60%, in particular at least 70% and in the best case at least 80% (cf. claim 6).

It goes without saying that the devices used for carrying out C-Stabilization 21 are suitable for this purpose.

C-stabilization 21 is performed in different ways, depending on the conversion residue partial stream. The conversion residue partial stream A (RS 12) is processed in such a way that the atmospheric carbon still contained in the conversion residue is stabilized as completely and permanently as possible under the secondary conditions of the lowest possible carbon loss. At the same time, the dry substance loss of the conversion residue 12 shall be as low as possible and the carbon content of the produced biochar mass 22 as high as possible. This can be achieved by carbonizing the conversion residue partial stream A (RS 12), wherein this carbonization 21 is preferably carried out by pyrolysis or torrefaction, more preferably by high-temperature pyrolysis and in particular by high-temperature pyrolysis which is carried out after slow heating. The conversion residue partial stream A (reference sign 12) is preferably exposed to a temperature of 150° C.-1,600° C. under oxygen deficiency, more preferably to a temperature of 500° C.-1,000° C. and in particular to a temperature of 600° C.-900° C. Preferably the reaction mass is exposed to the reaction temperature for more than 1 second, more preferably for more than 50 minutes and in particular for more than 500 minutes. Preferably, the molar H/C ratio of the produced, highly C-containing biochar/vegetable coals E (RS 22) is <0.8, more preferably <0.6, and/or its molar O/C ratio is <0.8, more preferably <0.4.

Biochar/vegetable coal E (RS 22) with fully stabilized atmospheric carbon increases the permanent humus content of the soil in an advantageous way with appropriate application because the atmospheric carbon contained in biochar/vegetable coal E can no longer react with atmospheric oxygen to form CO₂ or with hydrogen to form CH₄ for centuries and millennia.

Partial stream A (RS 12) preferably contains lignocellulose, more preferably wood and in particular straw. If it contains straw, the variant of the method according to the invention that is disclosed in FIG. 1 allows access to those ⅔ of the annual straw growth that had to remain in the field until now to secure the humus content of the soil.

The conversion residue partial stream B (RS 13) treated on the basis of C-stabilization 21 is processed in such a way that the atmospheric carbon still contained in the conversion residue is partially stabilized under the secondary conditions of the lowest possible carbon loss. At the same time, the dry mass loss of the conversion residue 13 shall be as low as possible and the carbon content of the produced biochar mass 23 as high as possible.

This can be achieved by carbonizing the conversion residue partial stream B (RS 13), wherein this carbonization 21 is preferably carried out by low-temperature pyrolysis or torrefaction, more preferably by hydrothermal carbonization HTC and in particular by low-temperature pyrolysis or torrefaction, which is carried out after rapid heating. Partial stabilization can also be achieved by the reaction parameters of a pyrolysis being aggressive, i.e. the heating rate to reaction temperature is rapid, the reaction temperature is relatively low and/or the reaction duration is relatively short. The conversion residue partial stream B (RS 13) is preferentially exposed under oxygen deficiency to a reaction temperature which is lower than the reaction temperature used for partial stream A (RS 12). The reaction mass is preferably exposed to the reaction temperature for a shorter period of time than the reaction time used for partial stream A (RS 12). Preferably, the molar H/C ratio of the produced, highly C-containing biochar/vegetable coals F (reference sign 23) is higher than that of the biochar/vegetable coals E (reference sign 22). Preferably, the molar O/C ratio of the produced, highly C-containing biochar/vegetable coals F (RS 23) is higher than that of the biochar/vegetable coals E (RS 22). Biochar/vegetable coal F (RS 23) with partially stabilized atmospheric carbon increases the nutrient humus content of the soil in an advantageous way with appropriate application because the atmospheric carbon contained in the biochar/vegetable coal F can no longer react with atmospheric oxygen to form CO₂ or with hydrogen to CH₄, at least proportionally for decades.

The conversion residue partial stream C (RS 14) treated on the basis of C-stabilization 21 is processed in such a way that the atmospheric carbon still contained in the conversion residue is not stabilized or is hardly stabilized at al under the secondary conditions of the lowest possible carbon loss. At the same time, the dry mass loss of the conversion residue 14 shall be as low as possible and the carbon content of the produced biochar mass 24 as high as possible. This can be achieved by carbonizing the conversion residue partial stream C (RS 14), wherein this carbonization 21 is preferably carried out by means of only short low-temperature pyrolysis or torrefaction, more preferably by means of short and/or low-pressure hydrothermal HTC carbonization and in particular by means of short low-temperature pyrolysis or torrefaction, which is carried out after very rapid heating. Non-stabilization can also be achieved by the reaction parameters of a pyrolysis being very aggressive, i.e. the heating rate to reaction temperature is very fast, the reaction temperature is very low and/or the reaction time is very short. The conversion residue partial stream C (RS 14) is preferably exposed under oxygen deficiency to a reaction temperature which is lower than the reaction temperature used for partial stream B (RS 13). Preferably, the reaction mass is exposed to the reaction temperature for a shorter period of time than the reaction time used for partial stream B (RS 13). Preferably, the molar H/C ratio of the produced, highly C-containing biochar/vegetable coals G (RS 24) is higher than that of the biochar/vegetable coals F (RS 23). Preferably, the molar O/C ratio of the produced, highly C-containing biochar/vegetable coal G (RS 24) is higher than that of the biochar/vegetable coals F (RS 23). Biochar/vegetable coal G (RS 24) with unstabilized atmospheric carbon increases the OPS or OSS content of the soil in an advantageous way with appropriate application because the atmospheric carbon contained in biochar/vegetable coal G is used as food or energy supplier for the soil flora and fauna at least proportionally for years.

The product of method step 21 (carrying out C-stabilization) are thus the biochar masses (BCM) 22 to 24. These have different properties as described above. Preferably, these BCM 22 to 24 are produced at least proportionally from straw-containing conversion residues (cf. claim 11).

The application of fresh, untreated biochar/vegetable coal or fresh, untreated biocoke can lead to the effect of temporary nitrogen immobilization and/or immobilization of other micro- and macronutrients, in particular if the biochar/vegetable coal or biocoke has been produced at low temperatures and/or by the HTC process. As shown above, this effect is due to inter alla the binding of the NH₄ ion and the resulting reduction of nitrification and increased soil respiration. In order that the fresh biochar/vegetable coals or the fresh biocoke 22 to 24 does not remove any nutrients from the topsoil after incorporation thereinto and immobilizes them, the biochar masses E to G produced are enriched in method step 25 (quenching/loading with nutrients) even before they are mixed to a biochar mixture H with exactly the same organic nutrients contained in the cereal plant (the enrichment with organic nutrients can also take place immediately after their mixing to a biochar mixture H).

Method step 25 preferably involves loading with nitrogen compounds, more preferably enrichment with organic nitrogen compounds. This prevents any (short-term) N-immobilization that may occur during method step 34.

The loading of the biochar masses E to G (RS 22 to 24) with nutrients can be BCM-specific by quenching the hot torrefied or pyrolyzed biochars/vegetable coals/biocokes separately with process water 17, which was extracted from the conversion residues 12 to 14 in method step 16, preferably together with nutrients, more preferably with the very nutrients that have previously been lost to the soil on which the biochars/vegetable coals are applied by the previous cultivation of the biomass from which the biochars/vegetable coals originate. Optionally, the process water 17 and/or the nutrients extracted from conversion residues 12 to 14 in method step 16 can be supplemented or replaced in this method step 25 by quenching or mixing the biomass coals 22 to 24 with a selection from the following nutrient-containing aqueous suspensions: slurry, percolate, swill, liquid residues from anaerobic fermentation, stillage from ethanol production, urine, seepage water from silages, process water (possibly processed or purified), liquid fermentation mass, permeate, more liquid phase of dehydration, more solid phase of dehydration, any phase of separation, suspensions prepared with mineral fertilizers, suspensions containing other nutrients and similar suspensions (cf. claim 8).

The loading of the biochar masses E to G (RS 22 to 24) with nutrients or the quenching of the biochar masses E to G (RS 22 to 24) with process water 17 is carried out with suitable devices which are previously known from the relevant prior art, preferably with tanks, containers and mixing devices (cf. claims 35 and 36).

If the amount of heat contained in the hot biochar/vegetable coals/bio coke 22 to 24 is less than the amount of heat required to evaporate the water supplied for quenching (only the water evaporates during quenching, the organic nutrients dissolved in the water remain in the biochar/vegetable coal mixture), the biochar/vegetable coals/biocoke 22 to 24 or the biochar/vegetable coal mixture H 26 become wet again, otherwise they remain dry. Preferably, only enough liquid (process water 17 or fresh water) is used for quenching 25 to keep the quenched biochar/vegetable-coal/biocoke dry.

If necessary, and if necessary at all, the quenched biochar/vegetable coals 22 to 24 can be dried (not shown in FIG. 1), preferably at low temperature. The low temperature drying known to persons skilled in the relevant art is advantageous because, in contrast to high temperature drying, no harmful dioxins and furans are formed. Corresponding devices are previously known from the relevant prior art. Such a need exists in particular if fungus formation and spontaneous combustion shall be prevented and/or if the transport weight of the biochar/vegetable coal mixture loaded with nutrients shall be reduced. Low-temperature drying is preferably carried out at a DS content of at least 86% since fungus formation can only be prevented from this DS content on.

The produced biochars/vegetable coals/biocokes can be quenched individually or as a coal mixture H (RS 26) or as a coal conversion residue mixture I (RS 27) or loaded with nutrients, in particular with N-containing nutrients. The corresponding devices previously known from the relevant prior art only have to be arranged or switched accordingly (cf. claim 36).

If the aim of the downstream biochar additions 32/33/34 is to bind or immobilize the N-surplus in the (agriculturally used soil), the biochar masses E to G (RS 22 to 24) are not loaded with nutrients in method step 25. Quenching can then either be completely omitted (and thus the entire method step 25) or it is carried out with purified process water 17 or with fresh water.

In an advantageous embodiment of the invention (not shown in FIG. 1), the biochar masses E to G (RS 22 to 24) produced by means of quenching/loading 25 can be stored in a silo, container, bunker or similar device previously known from the relevant prior art, preferably sorted by type, until they are needed. This need can arise from downstream mixing 26, from downstream mixing 27, from downstream pelletization/briquetting 28, from downstream filling in BigBags 30 or from downstream loose distribution, e.g. by tank trucks via regional interim storage facilities 31.

In method step 26 (mixing to form a biochar mixture H), the up to three biochar masses E to G (RS 22 to 24) are mixed in any combination and with any proportions to form a biochar mixture H (RS 26). The biochar mixture 26 can also consist of only one of the three biochar masses E to G (RS 22 to 24) (cf. claim 10). The biochar masses E to G (RS 22 to 24) can, but do not have to, be quenched beforehand with process water. Likewise, the biochar masses E to G (RS 22 to 24) can have been, but do not have to be, loaded with nutrients before being mixed together. According to the invention, special designer biochar mixtures with different properties can be produced by such mixing. Thus, method step 26 makes it possible to adapt the subsequent biochar/vegetable coal application 33/34 to the area-specific demand for OPS, OSS, nutrient humus, permanent humus and/or organic carbon.

The shares of the up to three biochar masses E to G (RS 22 to 24) in the biochar mixture H (RS 26) can each be between 0% and 100% under the natural secondary condition that the sum of the shares does not exceed 100% (cf. claim 10).

Preferably the biochar mass E (RS 22) has a share of >1% of the total coal mixture H (RS 26), more preferably a share of >50% and in particular a share of >75%.

The biochar mass F (RS 23) preferably has a share of <99% of the total coal mixture H (RS 26), more preferably a share of <50%, in particular a share of <25% and at best a share of <10%.

Preferably, the coal mixture H (RS 26) is produced at least proportionally from straw-containing conversion residues (cf. claim 11).

A partial stream of the biochar mixture H (RS 26) produced in the process step Mixing 26 can be stored in a silo 29, container, bunker or similar device known from the relevant prior art until it is needed. This need can be based on filing in BigBags 30 (or other containers), loose distribution e.g. by tank trucks via regional interim storage facilities 31, pelleting/briquetting 28 (not shown in FIG. 1) or mixing 27 (not shown in FIG. 1). The share of the partial stream in the total biochar mixture H stream can be between 0% and 100%.

For the division into the BCM partial streams E to G, mixing 26, mixing 27 and the intermediate storage in silo 29, suitable devices previously known from the relevant prior art are used (cf. claim 36).

It is also possible that the mixing 26 of the up to three biochar masses E to G (RS 22 to 24) is only carried out on the occasion of application 32 (not shown in FIG. 1). For this purpose, the application and distribution devices (fertilizer spreaders, solid manure spreaders, slurry spreaders and similar distribution devices previously known from the relevant prior art) consist of up to three departments in which the type-pure biochar masses E to G (RS 22 to 24) are located. Appropriate sensors previously known from the relevant prior art, installed in front of the distribution devices in the direction of travel (e.g. on the tractor), measure the soil content, preferably the topsoil, of OPS, OSS, nutrient humus, permanent humus, organic carbon, nitrogen and/or other substances during application 32 and the biochar-mass-specific additions are then made according to the resulting need or in such a way that the set target value is reached. The same is possible by including the conversion residue D (RS 15). In this case, the up to three biochar masses E to G (RS 22 to 24) and the one conversion residue D (RS 15) are spread and applied from up to four departments.

In method step 27 (mixing to form a biochar conversion residue mixture I), the biochar mixture H (RS 26) and the conversion residue D (RS 15) are mixed in any proportions to form a biochar conversion residue mixture I (RS 27). The biochar conversion residue mixture I 27 can also consist of only one of the three biochar masses E to G (RS 22 to 24) or only of the biochar mixture H (RS 26) (see claim 10). The biochar conversion residue mixture 127 can, but does not have to, be quenched beforehand with process water 17. Likewise, the biochar conversion residue mixture I 27 or its components can have been, but do not have to be, loaded with nutrients before their production. According to the invention, special designer biochar mixtures with different properties can also be produced by this mixing 27. Thus, method step 27 allows an even better adaptation of the subsequent biochar/vegetable coal application 33/34 to the area-specific need for OPS, OSS, nutrient humus, permanent humus and/or organic carbon.

The shares of the up to three biochar masses E to G (RS 22 to 24) and/or the biochar mixture H (RS 26) in the biochar conversion residue mixture I (RS 27) can each be between 0% and 100% under the natural secondary condition that the sum of the shares does not exceed 100% (cf. claim 10).

Preferably the biochar conversion residue mixture I (RS 27) is produced at least proportionally from straw-containing conversion residues (cf. claim 11).

In the case of mixing 27, suitable devices previously known from the relevant prior art are used for this purpose (cf. claim 36).

It is also possible that the mixing 27 takes place only on the occasion of application 32 (not shown in FIG. 1). For this purpose, the spreading and distribution devices (fertilizer spreaders, solid manure spreaders, slurry spreaders and similar distribution devices previously known from the relevant prior art) consist of up to four departments in which the type-pure biochar masses E to G (RS 22 to 24) and the conversion residue D (RS 15) or the biochar mixture H (RS 26) and the conversion residue D (RS 15) are located. Appropriate sensors previously known from the relevant prior art and installed in front of the distribution devices in the direction of travel (e.g. on the tractors), measure the content of the soil, preferably the topsoil, of OPS, OSS, nutrient humus, permanent humus, organic carbon, nitrogen and/or other substances during application 32 and the biochar-mass-specific additions are then made according to the resulting need or in such a way that the set target values are reached.

In an advantageous embodiment of the example of the method and system according to the invention, shown in FIG. 1, the transport suitability of the produced biochar mixture H (RS 26) and/or of the biochar conversion residue mixture I (RS 27) is increased and/or its downstream application 32 is facilitated by method step 28 (pelletizing). The pelletization is carried out as it is previously known from practice and the relevant prior art, i.e. It can comprise a selection of the following sub-processes: drying, comminution, evaporation, pressing, cooling, conveying, warehousing, storage. Pelletizing 28 is carried out using appropriate devices and systems previously known from the relevant prior art.

The biochar/vegetable coal pellets or biocoke pellets produced by pelletizing 28 can be stored in a silo 29, container, bunker or similar device previously known from the relevant prior art until they are needed. This need can arise from the downstream filling in BigBags 30 (or other containers) or from downstream bulk distribution, e.g. by tank trucks via regional interim storage facilities 31. The interim storage of the biochar/vegetable coal pellets or biocoke pellets in silo 29 and all associated upstream and downstream sub-processes (first conveying, warehousing, storage, outplacement, second conveying, etc.) are carried out with suitable devices and systems previously known from the relevant prior art.

As shown in FIG. 1 by dashed lines leading from reference sign 27 to reference signs 30 and 31, it is also possible that the biochar-conversion residue mixture I (RS 27), which can consist of only the biochar mixture H (RS 26) or only one of the biochar masses E to G (RS 22 to 24) (see above), is not pelletized and is loosely filled in BigBags in subsequent method step 30 or loosely distributed in subsequent method step 31.

The biochar-conversion residue mixture I (RS 27), which may have been pelletized in method step 28 and can consist only of the biochar mixture H (RS 26) or only of one of the biochar masses E to G (RS 22 to 24) (see above), is preferably filled in BigBags in method step 30 but can also be filled in bags, containers and similar receptacles. It is also possible that the biochar-conversion residue mixture I (RS 27) is comminuted, preferably ground, prior to filling 30. This is advantageous if the biochar-conversion residue mixture I (RS 27) shall be spread together with other media, such as slurry, solid manure, fermentation residue, swill, fertilizer, lime, etc., on the (agricultural and/or forestry) areas. The degree of fineness of the comminution depends on the needs of the customer (farmer), it can comprise a particle length from 0.1 mm to 100 mm. Comminution and filling 30 are carried out using appropriate devices previously known from the prior art.

In method step 31, the possibly pelletized biochar-conversion residue mixture I (RS 27), which is filled in BigBags and can consist of only the biochar mixture H (RS 26) or only one of the biochar masses E to G (RS 22 to 24) (see above), is distributed to regional interim storage facilities, preferably by rail, ship and/or truck. The biochar-conversion residue mixture I (RS 27) filled in BigBags can also be delivered directly to the end consumers (farmers).

In an advantageous variant of the embodiment of the invention, shown in FIG. 1, the possibly pelletized biochar mixture H (RS 26) and/or the possibly pelletized biochar-conversion residue mixture I (RS 27) can also be distributed in bulk to the intermediate storage facilities and/or to the end consumers. For this purpose, suitable devices previously known from the relevant prior art are used, preferably tank trucks, designed for the transport of pellets or powdery products, such as cement or flour.

In method step 32, the loose or packaged biochar-conversion residue mixture I (RS 27), which can also consist only of the biochar mixture H (RS 26) or only of one of the biochar masses E to G (RS 22 to 24) (see above), is delivered directly from the biomass conversion plant or from one of the regional intermediate storage facilities to the agricultural or forestry enterprise and filled, with or without intermediate storage, in devices suitable for distributing the biochar-conversion residue mixture I (RS 27), the biochar mixture H (RS 26) and/or at least one of the biochar masses E to G (RS 22 to 24) on the areas into which these biochars/vegetable coals or mixtures shall be incorporated. These can be all devices previously known from the relevant prior art, preferably fertilizer spreaders or solid manure spreaders.

These biochars/vegetable coals can also be mixed with solid fertilizers, solid manure or other substances so that the fertilizer spreaders or sold manure spreaders load and spread appropriate mixtures. If these other substances are liquid, the application of the biochars/vegetable coals or mixtures can also be carried out with slurry spreaders or devices having equal functions. In the latter case, it can be advantageous to comminute the biochars/vegetable coals or mixtures beforehand to such a degree of fineness that the slurry distributors or devices having equal function do not clog.

The biochars/vegetable coals or mixtures are applied on agricultural and forestry land after loading the distribution devices as previously known from the relevant prior art or from practice.

In method step 33, the biochars/vegetable coals or corresponding mixtures spread on the agricultural or forestry land are worked into the sol, preferably into the topsoil. This incorporation is carried out as previously known from the relevant prior art and practice, preferably by ploughing, cultivating or harrowing, with devices previously known from the relevant prior art, preferably by means of tractor-drawn ploughs, cultivators, harrows or similar devices.

In an advantageous variant of the embodiment of the invention, shown in FIG. 1, the biochars/vegetable coals incorporated into the soil, preferably into the topsoil, or the corresponding mixtures (RS 22 to 24, 26 and 27) are at least partly produced from lignocellulose-containing input materials, preferably from straw. More preferably, these input materials were exposed to high reaction temperatures. The biochars/vegetable coals produced from conversion residues containing straw preferably have a pH of >7.0, more preferably >8.5 and in particular >10.0. Preferably, the biochar/vegetable coal/biocoke produced from conversion residues containing straw is applied to acidic soils (cf. claim 11).

In an advantageous embodiment of the method shown in FIG. 1, unloaded stabilized biochars/vegetable coals/biocokes are incorporated into overfertilized and/or sandy soils to reduce overfertilization and/or nitrogen wash. Preferably, 0.1 to 5000 t biochar/vegetable coal/biocoke dry mass are applied per hectare, more preferably 1 to 1000 t biochar/vegetable coal/biocoke dry mass, in particular 10 to 500 t biochar/vegetable coal/biocoke dry mass and at best 20 to 100 t biochar/vegetable coal/biocoke dry mass.

Preferably, at least 5 t biochar/vegetable coal/biocoke per hectare and 100 years are incorporated into the soil, more preferably at least 50 t biochar/vegetable coal/biocoke per hectare and 100 years and in particular at least 100 t biochar/vegetable coal/biocoke per hectare and 100 years (cf. claim 12).

In another advantageous embodiment of the method shown in FIG. 1, the biochar/vegetable coal mixtures produced are applied with a high proportion of pyrolysis coals prior to the cultivation of cereal crops, preferably with a proportion of >50% and in particular with a proportion of >75%.

Preferably, the biochar-conversion residue mixture I (RS 27) incorporated into the soil, preferably into the topsoil, is produced at least proportionally from straw-containing conversion residues (cf. claim 11).

In an advantageous embodiment of the method shown in FIG. 1, so much of the produced biochar/vegetable coal-conversion residue mixture I (RS 27) is worked into the soil, preferably into the topsoil, that the proportion of biomass growth, preferably the proportion of straw growth, which had to remain on the fields before the method was used to maintain the humus content of the soil, can be reduced, and thus increased access to the biomass growth, preferably to the straw growth, becomes possible.

Preferably, the increased access based on the total biomass growth or to the total straw growth is >0.1% points, more preferably >30% points, in particular >50% points and in the best case >75% points (cf. claim 12).

In an advantageous embodiment of the method and system, shown in FIG. 1, at least a portion of the atmospheric carbon-containing biochar mixture H (RS 26) is not incorporated into agricultural or forestry soil, but is sequestered/end stored in geological formations, in stagnant waters, in aquifers or in the ocean, in soils not or no longer used for agriculture or forestry, or in bogs, desert soils, permafrost soils (cf. claim 7).

In method step 34, the incorporated biochars/vegetable coals or the corresponding mixtures are activated. This consists of securing, preferably improving, soil quality. This is achieved by securing, preferably increasing, the OPS or OSS content, the nutrient humus content, the permanent humus content and/or the content of organic carbon. The ways in which this can be done are described above. In order to avoid repetitions, reference is made to the above explanations.

The biochar/vegetable coal mixtures provided by the method and system of FIG. 1 are preferably used to improve at least one of the yield limiting soil properties.

In the sense of the invention, however, the desired main effect of method steps 33/34 is that atmospheric carbon is permanently removed from the atmosphere of the earth as part of a fuel or heating medium or combustion material production process. This decarbonization prevents atmospheric carbon from reacting (again) with atmospheric oxygen to form CO₂ or with hydrogen to form CH₄ for millennia. Accordingly, the GHG emission value of the product of the method according to the invention, the energy carrier mix 9 (which can also consist only of the produced energy carrier 5 or the energy carrier mix 7), is improved relative to the GHG emission value of its fossil counterpart, preferably to such an extent that no GHG emissions are associated with the production, distribution and use of the energy carrier mix 9, more preferably to such an extent that after the production, distribution and use of the energy carrier mix 9 there are fewer greenhouse gases or GHG quantities in the atmosphere of the earth than before.

The embodiment of the invention described in FIG. 1 can be modified in many ways. For example, individual method steps can be omitted without changing the end products (EC-Mix 9 and/or the effect in the soil & in the atmosphere of the earth 34). In the following, some variants are listed with reference to the embodiment of FIG. 1 that describe circumstances in which individual method steps and accordingly the use of corresponding devices can be omitted. These embodiments are not all-encompassing; for a person skilled in the art who is aware of the invention, further circumstances are obvious, in the presence of which some individual method steps 1 to 3, 6 to 9, 11 to 19 and 22 to 34 can be obsolete or are not absolutely necessary.

Briquetting 19, for example, can also be carried out without previous disintegration 18. This is also the case with pelletizing 19 if the conversion residues A to C (RS 12 to 14) to be pelletized are small enough. Dehydration 16 is also not absolutely necessary, e.g. If, for example, an HTC shall be carried out in method steps 20/21 or if the conversion residues A to C (RS 12 to 14) have a DS content sufficient for efficient pyrolysis or torrefaction. Nutrient extraction 16 can be dispensed with, as can pelletizing 19, for example if carbonization processes are used in method steps 20/21 that function without pelletized input materials. If only one type of biochar mass E, F or G (RS 22 to 24) shall be produced and no conversion residue D (RS 15) is required, e.g. separation 11 is also superfluous. Pre-treatment 3 of the input materials 1/2 is also not absolutely necessary, e.g. If high conversion efficiencies shall not be achieved in the conversion 4 and/or the focus is more on ensuring that as large a proportion as possible of the atmospheric carbon contained in the at least one input material remains in conversion residue 10 in order to achieve as high a decarbonylation effect 34 as possible. Harvest and collection 2 can be omitted, for example, if at least one input material is produced anyway and therefore does not have to be harvested or collected. If the substance 1/2 to be used is given, e.g. method step 1 is superfluous. The mixtures 7 and/or 9 do not necessarily have to occur either, the desired GHG effect still occurs. Furthermore, under the circumstances described above, loading with nutrients 25 can be omitted. In the case of C-stabilization by carbonization of the conversion residues, for example, quenching 25 can be omitted if there is a corresponding alternative to cooling hot biochar/vegetable coal, e.g. cooing with air.

The mixtures 26 and 27 can be omitted if only one biochar mass E, F or G (RS 22 to 24) shall be produced. Filling in BigBags 30 can be omitted if e.g. filing in bags is carried out or if the produced biochar/vegetable coal mixture shall be distributed in bulk. Distribution to regional intermediate storage facilities 31 becomes obsolete if e.g. the end customer is supplied directly or if he collects the biochar/vegetable coal mixture himself at the biomass conversion plant. The loading of application devices and the application 32 as well as the incorporation into the field topsoil become superfluous if the biochar mixture H (RS 26) produced is sequestered elsewhere than in the soil used for agriculture or forestry.

The embodiment of the invention shown in FIG. 1, its sub-variants described above and the above-listed examples of shorter embodiments and other embodiments of the invention, which result from the entire above description, the claims and, where applicable, the reference signs, or which are obvious to a person skilled in the art after becoming aware of the invention, can also be extended in many ways. For example, it is possible to use devices and/or methods for the recuperation of process heat, for heat exchange and/or for heat recirculation, which can preferably include components that function according to the countercurrent principle (see claims 26 and 32). It is also possible, for example, top provide one or more conveyances and/or one or more intermediate storage between the individual method steps or between the individual devices by means of suitable devices previously known from the relevant prior art. These conveyances and/or intermediate storage can include the sub-methods of a first conveyance, warehousing, storage, taking out of storage and second conveyance. It is also possible, for example, that the biochar masses, biochar/vegetable coal mixtures, biochar conversion residue mixtures produced or the soil are provided with additives as known from the relevant prior art or from horticultural practice and/or plant cultivation before or after incorporation of the biochar (mixtures) produced. Furthermore, it is possible to put the produced biochar masses, biochar/vegetable coal mixtures, biochar conversion residue mixtures to uses previously known from the relevant prior art other than the incorporation into the soil 33. Such additional measures, as well as the devices used for them, are considered to be the usual current or future specialist knowledge of a competent person skilled in the art and shall also be protected.

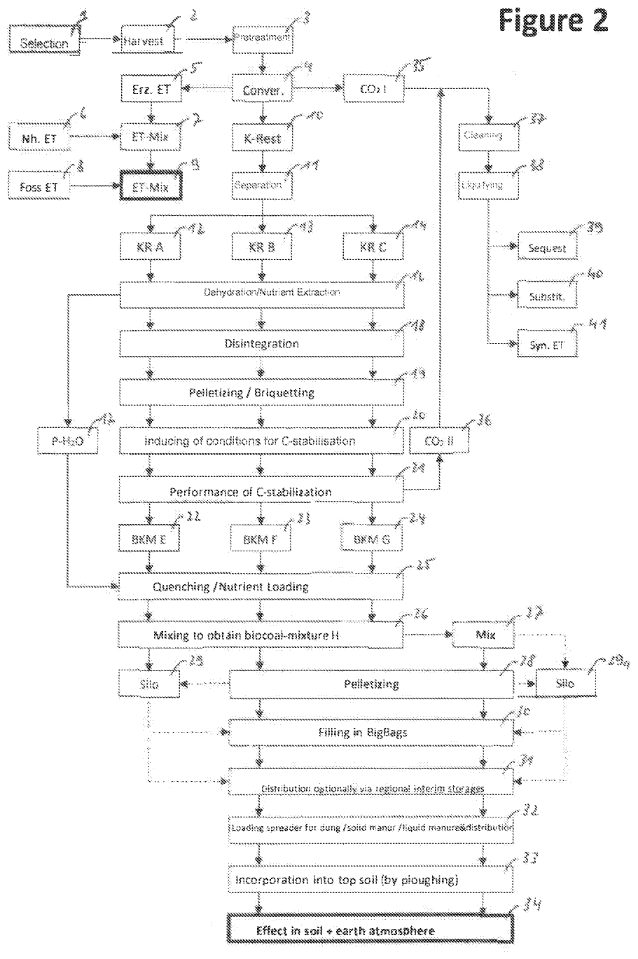

FIG. 2 shows a schematic diagram of a further example of the method and system according to the invention for reasons of a clearer representation without the conversion residue D and its use indicated in FIG. 1. It goes without saying that the embodiment shown in FIG. 2 can also be carried out with conversion residue D and the use thereof, as shown in FIG. 1.

A first recuperation of atmospheric carbon dioxide (CO₂ I) with reference sign 35 and a second recuperation of atmospheric carbon dioxide (CO₂ II) with the reference sign 36 are added in FIG. 2. CO₂-I (RS 35) can occur as a by-product or residue of conversion 4. The recuperation of CO₂-I (RS 35) is carried out on the occasion of this (first) conversion 4 of the selected biomass 1/2 into a produced energy carrier 5. Such recuperation is e.g. possible in the production of bio-ethanol and in the processing of biogas into bio-methane. For the recuperation of CO₂, suitable devices previously known from the relevant prior art are used, preferably pipelines with valves, especially pressurized gas pipelines.

The second recuperation of atmospheric carbon dioxide (CO₂ II) takes place in method step 21 on the occasion of the chemical-physical stabilization of atmospheric carbon. CO₂-II (RS 36) can occur as a by-product or as a residue of C-stabilization 21. Such CO₂ recuperation 36 is e.g. possible in the carbonation of biomass, especially in the pyrolysis of biomass. The combustion of pyrolysis gas produces a flue gas with a high CO₂ content.

The recuperated CO₂ I (RS 35) and the recuperated CO₂ II (RS 36) are combined, purified (RS 37), liquefied (RS 38) geologically sequestered (RS 39), used as a substitute for fossil CO₂ (RS 40) or for the production of CO₂-based energy carriers (RS 41), preferably for the production of syn-methane (cf. claim 13). These uses are advantageous because, due to the resulting decarbonization effects, the GHG emission values of the produced energy carrier 5 can be improved and consequently the admixture quantities of sustainable energy carrier 6 and/or fossil energy carrier 8 can be increased without affecting the GHG emission values of the energy carrier mixtures 7 and 9.

In order to implement this, devices previously known from the relevant prior art are used which are suitable for recuperating, liquefying, purifying, processing, storing, transporting (preferably in a liquid state of aggregation) atmospheric carbon doxide (CO₂) produced in the method according to the invention, delivering it to industry, introducing it into geological formations, converting it into CO₂-based fuel, heating medium or combustion material, performing a combination of these functions (cf. claim 37).

### LIST OF REFERENCE SIGNS (KS)

1 Selection of at least one biogenic input material containing atmospheric carbon or the selected input material itself
2 Harvesting/collection of the at least one biogenic input material (biomass) selected in 1 or the harvested/collected at least one input material itself
3 If necessary, pretreatment/disintegration of the at least one input material 2 or the pretreated input material itself
4 Single-stage or multi-stage conversion of the possibly pre-treated input material 3 into an energy carrier 5 containing atmospheric carbon 5
5 Sustainable energy carrier resulting from 4, preferably used as transport fuel
6 Sustainably produced energy carrier from another conversion process with a higher GHG emission value than energy carrier 5, which is used in particular as a transport fuel
7 Mixing the energy carrier 5 with another sustainable energy carrier 6 to form an energy carrier mixture 7, wherein the energy carrier 5 is preferably bio-methane, the other sustainable energy carrier 6 is preferably syn-methane produced from wind power and atmospheric CO₂
8 Fossil energy carrier (fuel, heating medium or combustion material), preferably CNG or LNG
9 Mixing of the energy carrier mix 7 with a fossil energy carrier 8 to form an energy carrier mix 9, the mixing of which preferably takes place in such a way that, after the production, distribution and use of the energy carrier mix according to life cycle analysis or stoichiometric analysis, the amount of greenhouse gas in the atmosphere of the earth (measured in tons of CO₂ equivalent) is the same or lower than after the production, distribution and use of an equal energy amount of the fossil counterpart of the energy carrier mixture, more preferably in such a way that, after the production, distribution and use of the energy carrier mix, a smaller amount of greenhouse gases is in the atmosphere of the earth than before, i.e. the energy carrier mix produced is GHG negative.
10 Recuperation of conversion residues (K residue) from 4 or the recuperated conversion residue itself
11 Distribution of the recuperated conversion residues CR
12 Conversion residue partial stream A
13 Conversion residue partial stream B
14 Conversion residue partial stream C
15 Conversion residue partial stream D
16 Dehydration by separation into a more solid and a more liquid phase, wherein the more liquid phase is used as process water, and/or extraction of organic nutrients
17 Process water, preferably containing nutrients
18 Disintegration
19 Pelletizing/briquetting
20 Generation of conditions which allow at least partial chemical-physical stabilization of the atmospheric carbon still contained in the conversion residue
21 Carrying out the chemical-physical stabilization of atmospheric carbon, preferably by carbonization, more preferably by pyrolysis or torrefaction
22 Resulting stabilized carbon, preferably contained in biochar/vegetable coal/biocoke (BC mass E)
23 Resulting partially stabilized carbon, preferably contained in biochar/vegetable coal/biocoke (BC mass F)
24 Resulting unstabilized carbon, preferably contained in biochar/vegetable coal/biocoke (BC mass G)
25 Loading the biochar/vegetable coal/biocoke masses E to G with organic nutrients, preferably with organic nutrients extracted from the conversion residue according to 16, more preferably by quenching the hot output of the pyrolysis/torrefaction devices with the more liquid phase obtained at 16
26 Mixing of the biochar/vegetable coal/biocoke masses E to G loaded according to 25 to form a biochar/vegetable coal/biocoke mixture H or the biochar/vegetable coal/biocoke mixture H itself
27 Mixing the biochar/vegetable coal/biocoke mixture H mixed according to 26 with the conversion residue partial stream D to form a biochar/vegetable coal/biocoke conversion residue mixture I or the biochar/vegetable coal/biocoke conversion residue mixture I itself
28 Pelletizing, where appropriate, the biochar/vegetable coal/biocoke mixture H obtained according to 26, which can also be only a biochar/vegetable coal/biocoke mass E to G, and/or the biochar/vegetable coal/biocoke conversion residue mixture I obtained according to 27
29 Possibly intermediate storage of the biochar/vegetable coal/biocoke masses E to G pelletized according to 28, of the biochar/vegetable coal/biocoke mixture H and/or the biochar/vegetable coal/biocoke conversion residue mixture I, preferably in silos 29
30 Filling of loose and/or pelletized biochars/vegetable coals/biocokes, preferably in BigBags
31 Distribution of biochars/vegetable coals/biocokes packed in BigBags, if applicable, preferably to regional distribution points
32 Loading agricultural/forestry fertilizer, slurry and/or solid manure spreading devices, if necessary after further intermediate storage in the agricultural or forestry companies and spreading of the biochars/vegetable coals/biocokes E to G or with biochar/vegetable coals/biocoke mixtures H or with biochar/vegetable coal/biocoke conversion residue mixtures I preferably together with fertilizer, solid manure and/or slurry
33 Incorporation d into agricultural soil, preferably by ploughing, more preferably by ploughing into the topsoil
34 Effect of biochars/vegetable coals/biocokes or biochar/vegetable coal/biocoke mixtures or biochar/vegetable coal/biocoke conversion residue mixtures incorporated into the agricultural soil, which differs in the soil and atmosphere of the earth depending on the biochar/vegetable coal/biocoke type (E to q) incorporated into the soil
35 Recuperation of atmospheric CO₂ I resulting from the single-stage or multi-stage conversion according to 4 of possibly pre-treated biomass 3 into a produced energy carrier 5
36 Recuperation of atmospheric CO₂ II, which is produced during (partial) stabilization 21 of atmospheric carbon
37 Purification of atmospheric CO₂ recuperated according to 35 and/or 36, if necessary,
38 Liquefaction of atmospheric CO₂ recuperated according to 35 and/or 36
39 Sequestration of atmospheric CO₂ in carbon sinks
40 Substitution of fossil CO₂ with atmospheric CO₂
41 Manufacture of synthetic fuel, heating medium or combustion material from atmospheric CO₂

We claim:

1. Method for converting biomass containing atmospheric carbon, preferably lignocellulose-containing biomass, more preferably straw, straw-containing input materials (e.g. solid manure) and/or wood, into GHG emission-reduced energy carriers, preferably biogas, bio-methane, pyrolysis gas, synthesis gas, bio-diesel, Fischer-Tropsch fuel, DME, bio-methanol or bio-ethanol, on the one hand, and chemically and physically stabilized atmospheric carbon, on the other hand, comprising the following steps:
(1) Single-stage or multi-stage conversion of atmospheric carbon-containing biomass into another energy carrier, preferably a GHG emission-reduced energy carrier, more preferably by anaerobic bacterial fermentation into biogas or bio-methane, by alcoholic fermentation into bioethanol or ligno-ethanol, by gasification into pyrolysis gas, by carbonization into carbonization gas, by transesterification into bio-diesel, by Fischer-Tropsch synthesis into FT-fuel (FT-diesel, FT-gasoline, FT-kerosene, FT-methanol), by methanol synthesis into bio-methanol, by dimethyl ether synthesis into DME,
(2) Generation of conditions that allow an at least partial chemical-physical stabilization of the atmospheric carbon still contained in the biomass conversion residues (e.g. digestion, fermentation, pyrolysis or synthesis residues),
(3) Conduction of the at least partial chemical-physical stabilization of the atmospheric carbon still contained in the biomass conversion residues.

2. Method according to claim 1, in which the atmospheric carbon is at least partially stabilized in such a way that it is degraded (mineralized) within a given period of time to less than 30%, preferably less than 20%, in particular less than 10% and at best less than 5% by the processes of soil respiration, weathering, aerobic rotting and/or reaction with atmospheric oxygen, wherein the given period of time can be a selection from the following periods: 10 years, 30 years, 100 years, 500 years, 1,000 years, 10,000 years, 100,000 years, >100,000 years.

3. Method according to claim 1, in which the loss of atmospheric carbon or the loss of conversion residue dry substance which occur during the at least partial chemical-physical stabilization of the conversion residues is a maximum of 99%, preferably a maximum of 60%, more preferably a maximum of 40% and in particular a maximum of 30%.

4. Method according to claim 1, in which organic nutrients still contained in the residues of the single-stage or multi-stage biomass conversion are at least partially removed from these residues before the chemical-physical treatment, preferably together with process water, more preferably by a selection from the methods of centrifuging, decanting, pressing, separation, filtration, reverse osmosis, combination of these method steps, and in particular by recirculation of process water into the process, and/or in which the residues from the single-stage or multi-stage biomass conversion are pelletized or briquetted before the chemical-physical treatment, preferably after dewatering to >35% DS, in particular after dewatering to >50% DS and in particular after dewatering to >60% DS.

5. Method according to claim 1, in which the at least partial chemical-physical stabilization of the atmospheric carbon still contained in the residues of the single-stage or multi-stage biomass conversion is effected by a chemical-physical treatment of these residues, preferably by a thermal or thermo-chemical carbonization of these residues to form biochar/vegetable coal/biocoke, more preferably by a selection from the following thermo-chemical carbonization processes: pyrolysis, carbonization, torrefaction, hydrothermal carbonization (HTC), vapothermal carbonization, gasification and any combination of these treatment methods, and in particular by pyrolysis or torrefaction of dehydrated (dewatered) residues from the single-stage or multistage biomass conversion, wherein the dewatering preferably takes place to >35% dry substance (DS), more preferably >50% DS and in particular >60% DS.

6. Method according to claim 5, in which the dry mass loss which occurs during the carbonization of the conversion residues from the single-stage or multistage biomass conversion is a maximum of 99%, preferably a maximum of 60%, more preferably a maximum of 40% and in particular a maximum of 30% and/or in which the carbon content of the biochar/vegetable coal/biocoke produced is at least 20%, preferably at least 40%, more preferably at least 60%, in particular at least 70% and in the best case at least 80%, and/or in which the molar H/C ratio of the biochar/vegetable coal/biocoke produced is <0.8, preferably <0.6, and/or the molar O/C ratio of the biochar/vegetable coal/biocoke produced is <0.8, preferably <0.4.

7. Method according to claim 5, in which at least a portion of the biochar/vegetable coal/biocoke containing atmospheric carbon is sequestered (disposed of permanently) in an additional method step in the soil (geological formations), in stagnant waters, in aquifers or in the ocean, preferably in agricultural or forestry soils, more preferably in soils which are not or no longer used for agriculture or forestry, and in particular in bogs, desert or permafrost soils.

8. Method according to claim 5, in which the biochar/vegetable coal/biocoke containing atmospheric carbon is loaded (mixed) with nutrients prior to incorporation into soil formations, preferably with organic nutrients, more preferably with organic nutrients, which are contained in a selection of the following aqueous suspensions: slurry, percolate, swill, stillage from ethanol production, liquid residues from anaerobic fermentation, urine, seepage water from silages, (possibly treated or purified) process water, liquid fermentation mass, permeate, more liquid phase of dehydration, more solid phase of dehydration, any phase of separation, suspensions containing other nutrients and similar suspensions, and in particular with organic nutrients removed from the conversion residues to be carbonized before the carbonization of the conversion residues.

9. Method according to claim 5, in which the recuperated proportion of the conversion residues stream leaving the method step of the single-stage or multi-stage biomass conversion is divided into up to four partial streams before its carbonization in an additional method step, namely into the first partial stream “production of stabilized pyrolysis coal”, the second partial stream “production of partially stabilized torrefaction or HTC coal”, the third partial stream “production of unstabilized biochar/vegetable coal/biocoke” and the fourth partial stream “non-carbonized conversion residues”, wherein each of the partial streams can represent between 0% and 100% of the total stream (each partial stream can represent both the total stream and zero), wherein, in the method steps following the stream distribution, that is carried out with the respective partial stream which is indicated by the partial stream designation and/or wherein the first partial stream “production of stabilized pyrolysis coal” preferably has a proportion of >1% of the total stream, more preferably a proportion of >25%, in particular a proportion of >50% and in the best case a proportion of >75%.

10. Method according to claim 9, in which the up to four products which are produced in the method steps following the conversion residue distribution are produced in parallel or in series and/or in which they are mixed in any selection or combination to form a biochar/vegetable coal/biocoke mixture or to form a biochar/vegetable coal/biocoke conversion residue mixture, wherein the proportions of the up to four products can each be between 0% and 100% under the self-evident secondary condition that the sum of the proportions does not exceed 100%.

11. Method according to claim 5, in which the produced biochar/vegetable coal/biocoke, the biochar/vegetable coal/biocoke mixture or the biochar/vegetable coal/biocoke conversion residue mixture are produced at least in part from straw-containing conversion residues and/or in which the biochar/vegetable coal/biocoke, the biochar/vegetable coal/biocoke mixture or the biochar/vegetable coal/biocoke conversion residue mixture have a pH of >7.0, preferably a pH of >8.0, more preferably a pH of >9.0 and in particular a pH of >10.0, and wherein these basic products are preferably incorporated into acidic soils.

12. Method according to claim 1, in which chemically and physically stabilized atmospheric carbon, preferably atmospheric carbon carbonized at least partially into biochar/vegetable coal/biocoke, is incorporated into areas used for agriculture or forestry (arable soils, fields, forests, banks), preferably at least 5 t of biochar/vegetable coal/biocoke per hectare and 100 years, more preferably at least 50 t biochar/vegetable coal/biocoke per hectare and 100 years and in particular at least 100 t biochar/vegetable coal/biocoke per hectare and 100 years, and this C-application contributes to maintaining or increasing the humus content of the soil, preferably the C-containing humus content of the soil, more preferably the active nutrient humus content in the soil, in particular the passive permanent humus content in the soil, such that the proportion of biomass growth, preferably the proportion of straw growth, which had to remain in the fields prior to the application of the method for maintaining the humus content of the soil, can be reduced and thus increased access to the biomass growth, preferably to the straw growth, becomes possible, wherein the increased access based on the total biomass growth or the straw growth is preferably >0.1%-points, more preferably >30%-points, in particular >50%-points and in the best case >75%-points.

13. Method according to claim 1, in which atmospheric carbon dioxide (CO₂) produced as a by-product, waste or residue is subjected to a selection of the following method steps: recuperation, purification, liquefaction, processing, sequestration (in geological formations, such as crude oil or natural gas reservoirs), substitution of fossil CO₂, production of CO₂-based energy carriers (syn-methane, syn-methanol), combination of these method steps.

14. Method according to claim 1, in which the energy carrier produced, which is preferably a selection of biogas, bio-methane, pyrolysis gas, synthesis gas, bio-diesel, bio-kerosene, Fischer-Tropsch fuel, bio-methanol, DME or bio-ethanol, is processed in such a way that it can be used as a fuel, heating medium or combustion material, preferably as a transport fuel, more preferably as a road fuel.

15. Method according to claim 1, in which, after the production, distribution and use of the energy carrier produced, which is preferably a fuel, more preferably a gas fuel and in particular bio-methane, there is a smaller amount of greenhouse gas in the atmosphere of the earth than after the production, distribution and use of an equal amount of energy of the fossil counterpart of the energy carrier produced, wherein mineral diesel fuel is the fossil counterpart for all diesel substitutes, mineral fuel for Otto engines (gasoline) the fossil counterpart for all substitutes of fuel for Otto engines, mineral kerosene the fossil counterpart for all kerosene substitutes, natural gas (CNG) the fossil counterpart for all natural gas substitutes, LNG the fossil counterpart for all LNG substitutes, LPG the fossil counterpart for all LPG substitutes and the weighted average of mineral fuel for Otto engines and mineral diesel the fossil counterpart for all other fuels, heating mediums and combustion materials.

16. Method according to claim 1, in which, after the production, distribution and use of the energy carrier produced there is a smaller amount of greenhouse gas in the atmosphere of the earth than before, i.e. the energy carrier produced is GHG-negative.

17. Method according to claim 1, in which the produced energy carrier (fuel, heating medium or combustion material) is mixed with a GHG-positive energy carrier (fuel, heating medium or combustion material), which is preferably a fossil counterpart of the produced energy carrier and more preferably a sustainable energy carrier, such that after the production, distribution and use of the produced energy carrier mixture there is a smaller amount of greenhouse gas in the atmosphere of the earth than after the production, distribution and use of an equal amount of energy of the fossil counterpart of the produced energy carrier, wherein mineral diesel fuel is the fossil counterpart for all diesel substitutes, mineral fuel for Otto engines (gasoline) the fossil counterpart for all substitutes of fuel for Otto engines, mineral kerosene the fossil counterpart for all kerosene substitutes, natural gas (CNGis the fossil counterpart for all natural gas substitutes, LNG is the fossil counterpart for all LNG substitutes, LPG is the fossil counterpart for all LPG substitutes and the weighted average of mineral fuel for Otto engines and mineral diesel the fossil counterpart for all other fuels, heating mediums and combustion materials.

18. Method according to claim 1, in which the generated energy carrier (fuel, heating medium or combustion material) is mixed with a GHG-positive energy carrier (fuel, heating medium or combustion material), which is preferably a fossil counterpart of the produced energy carrier and more preferably a sustainable energy carrier, such that after the production, distribution and use of the produced energy carrier mixture there is a smaller amount of greenhouse gas in the atmosphere of the earth than before, i.e. the energy carrier mixture is GHG-negative.

19. Method according to claim 17, in which mixing of the produced energy carrier is carried out with an energy carrier which is its fossil or its sustainable counterpart, wherein the mixing is preferably carried out in such a way that the resulting energy carrier mixture has a GHG emission value that is lower than the GHG emission value of the admixed energy carrier, and in particular in such a way that according to the life cycle analysis (WtW) or after stoichiometric analysis (TtW) the resulting energy carrier mixture has a GHG emission value which is less than/equal to 0.0 gCO₂-eq/kWh_{Hi} or less than/equal to 0.0 gCO₂-eq/MJ.

20. Method according to claim 1, in which the physical-chemical stabilization of the atmospheric carbon takes place under oxygen deficiency and/or at reaction temperatures of 100° C.-1600° C., preferably at reaction temperatures of 200° C.-1,200° C., more preferably at reaction temperatures of 300° C.-1.000° C., in particular at reaction temperatures of 350° C.-1,000° C. and in the best case at reaction temperatures of 400° C.-900° C., and/or in which the heating of the residue to be treated from the single-stage or multi-stage biomass conversion to reaction temperature takes longer than 1 second, preferably longer than 10 minutes, more preferably longer than 50 minutes and in particular longer than 100 minutes.

21. Method according to claim 1, in which the method step of the single-stage or multi-stage conversion of biomass is preceded by the method step of selecting and/or harvesting or collecting at least one biogenic input material containing atmospheric carbon, wherein this input material is preferably characterized in that the selection is made from the input material groups of cultivated biomass, straw (cereal straw, corn straw, rice straw and the like; pure or as part of silage), farm manure, solid manure containing straw (solid cow manure, solid pig manure, poultry manure, dry chicken dung, horse manure, etc.), straw-containing residues from mushroom cultivation, slurry, swill, fresh grass-like plants (ryegrass, switch grass, miscanthus, giant reed and catch crops before and after main crops) and silages from these grass-like plants, whole-plant corn cuttings and corn silage, whole-plant cereal cuttings and silage from cereal whole plants, cereal and corn grains, wood, waste, residues from biomass processing, by-product from biomass processing, cellulose-containing non-food material, waste paper, bagasse, grape marc and wine lees, lignocellulose-containing biomass, residual forest wood, landscape conservation material, roadside greenery, cereals and other crops with a high starch content, sugar plants, oil plants, algae, biomass fraction of mixed municipal waste, household waste, biowaste, biowaste from private households, biomass fraction of industrial wastes including materials from wholesale and retail, agricultural and food industries as well as the fishing industry and aqua industry, slaughterhouse waste, sewage sludge, waste water from palm oil mills, empty palm fruit bundles, tall oil pitch, crude glycerine, glycerine, bagasse, molasses, grape marc, wine lees, stillage from ethanol production, nut shells, husks, cored corn cobs, biomass fractions of wastes and residues from forestry and forest-based industries (bark, twigs, pre-commercial thinnings, leaves, needles, tree tops, sawdust, wood shavings, black liquor, brown liquor, fiber sludges, lignin, tall oil), other cellulose-containing non-food material, other lignocellulose-containing material, bacteria, used cooking oil, animal fats, vegetable fats or combinations thereof.

22. Method according to claim 1, in which an additional step of recuperating at least a portion of the residues from the single-stage or multi-stage biomass conversion is performed between the step of single-stage or multi-stage conversion of the biomass and the step of the at least partial chemical-physical stabilization of the atmospheric carbon still contained in the residues of the biomass conversion.

23. Method according to claim 1, in which the method step of the single-stage or multi-stage conversion of the biomass into another energy carrier consists of an anaerobic bacterial fermentation, which is preferably carried out according to the solid fermentation process, more preferably the process of solid fermentation in garage fermenters or plug-flow fermenters, and/or in which before, during or after the method step of the single-stage or multi-stage conversion of the biomass this biomass is provided with at least one suitable admixture previously known from the relevant prior art, preferably an admixture from the selection: lime, enzymes, enzyme-containing solutions, fungi, acids, lyes, yeasts, water, recycled process water, purified process water, filtered process water, ultra-filtrated process water, process water subjected to reverse osmosis, treated process water, acid-water mixtures, lye-water mixtures, percolate, silage seepage juices, slurry, micro-organisms, any cereal grain stillage from ethanol production, any residue from the production of ligno-ethanol, any by-product/residue from the production of pyrolysis or synthesis gas, any by-product/residue from FT-synthesis, any by-product/residue from DME synthesis, any by-product/residue from methanol synthesis, any sugar beet stillage from ethanol production, combination of two or more of these additives.

24. Method according to claim 1, in which the biomass is subjected, before or after the method step of the single-stage or multi-stage conversion of the biomass into another energy carrier, to comminution, preferably chopping or shredding, more preferably the comminution combination consisting of chopping or shredding and grinding, and in particular the comminution combination consisting of bale disintegration, chopping/shredding and grinding and/or in which the comminution takes place in one or more stages to an average final particle length of <20 cm, preferably to an average final particle length of <5 cm, more preferably to an average final particle length of <10 mm, in particular to a final particle length of <3 mm and in the best case to a final particle length of <1 mm.

25. Method according to claim 1, in which the biomass is subjected, before, during or after the method step of the single-stage or multi-stage conversion of the biomass into another energy source, to a treatment which consists of a selection of the following treatment methods: comminution, soaking/mixing/mashing in cold water or aqueous suspensions including lyes and acids, soaking/mixing/mashing in 20° C.-100° C. warm water or aqueous suspensions including lyes and acids, biological treatment with fungi, pressurization to >1 bar-500 bar, treatment with >100° C. hot water, treatment with saturated steam, treatment by thermal pressure hydrolysis, treatment by wet oxidation, treatment by extrusion, ultrasonic treatment, steam reforming treatment, steam explosion treatment, drying, treatment with process water, treatment with process heat, treatment with enzymes, combination of a selection of these treatment methods.

26. Method according to claim 1, in which process heat from a method step is recycled into the process, preferably by means of heat exchange functioning in counterflow, and/or into a warming or heating step of the process, more preferably process heat from a thermal or thermo-chemical treatment before or after the single-stage or multi-stage conversion of the biomass into a warming or heating step, in particular process heat from the method step of the chemical-physical stabilization of the atmospheric carbon still contained in the conversion residues into a warming or heating step and at best process heat from the thermal or thermo-chemical carbonization of the conversion residues into a warming or heating step.

27. System for performing at the method of claim 1, comprising
(a) devices for the single-stage or multi-stage conversion of biomass, preferably lignocellulose-containing biomass, more preferably straw-containing biomass, into a GHG emission-reduced energy carrier,
(b) devices for generating conditions allowing an at least partial chemical-physical stabilization of the atmospheric carbon still contained in the residues of the single-stage or multi-stage biomass conversion (digestion, fermentation, pyrolysis or synthesis residues and the like).

28. System according to claim 27, in which the devices for the single-stage or multi-stage conversion of biomass consist of suitable devices previously known from the relevant prior art, preferably a selection of the following devices: devices for the anaerobic bacterial fermentation of biomass into biogas and/or bio-methane, devices for the alcoholic fermentation of biomass into bio-ethanol or ligno-ethanol, devices for the gasification of biomass into pyrolysis gas and/or pyrolysis slurry, devices for the carbonization of biomass into carbonization gas (weak gas), devices for the transesterification of vegetable oils into bio-diesel (FAME), devices for the hydration of vegetable oils in HVO (mineral oil refineries), devices for the refining of vegetable oils in HVO (NesteOil process), devices for the gasification/pyrolysis of biomass to process gas, devices for the conversion of biomass-derived process gas to synthesis gas, devices for the synthesis of biomass-derived synthesis gas to a Fischer-Tropsch fuel (FT-diesel, FT-gasoline, FT-kerosene, FT-methanol and the like), devices for the synthesis of methanol from biomass-derived gases, devices for DME synthesis, any combination of these devices.

29. System according to claim 27, in which the devices for generating conditions allowing at least partial chemical-physical stabilization of the atmospheric carbon still contained in the biomass conversion residues, include appropriate devices previously known from the relevant prior art, preferably devices for the chemical-physical treatment of these residues, more preferably devices for the thermal or thermo-chemical carbonization of these residues to biochar/vegetable coal/biocoke, in particular a selection from the following devices for the thermo-chemical carbonization of biomass to biochar/vegetable coal/biocoke: pyrolysis devices, carbonization devices, torrefaction devices, hydrothermal carbonization (HTC) devices, vapothermal carbonization devices, gasification devices, any combination of these devices, wherein the carbonization devices for the conversion residues are preferably suitable for carrying out the carbonization under oxygen deficiency and/or at reaction temperatures of 100° C.-1600° C., more preferably at reaction temperatures of 200° C.-1.200° C., in particular at reaction temperatures of 300° C.-1,000° C., in an even better case at reaction temperatures of 350° C.-950° C. and in the best case at reaction temperatures of 400° C.-900° C.

30. System according to claim 27, in which the devices for the at least partial chemical-physical stabilization of atmospheric carbon are suitable for carbonizing the residues from the single-stage or multi-stage biomass conversion to such biochar/vegetable coals, or to such biocoke that their proportion of atmospheric carbon is preferably degraded (mineralized) within a certain period of time to less than 50%, more preferably to less than 20%, in particular to less than 10% and in the best case to less than 5% by the processes of soil respiration, weathering, aerobic rotting and/or reaction with atmospheric oxygen, wherein the certain period of time can be a selection from the following periods of time: 10 years, 30 years, 100 years, 500 years, 1,000 years, 10,000 years, 100,000 years, >100,000 years.

31. System according to claim 27, comprising devices suitable for extracting or separating at least a portion of the organic nutrients still contained in the residues of the single-stage or multi-stage biomass conversion and/or a portion of the water contained in the residues of the single-stage or multi-stage biomass conversion, wherein these devices preferably consist of a selection from the following devices: spinners, centrifuges, cyclones, decanters, presses, separators, screens, filtration devices, ultrafiltration devices, reverse osmosis devices, similar devices, combinations of these devices, and/or wherein these devices are more preferably suitable for dewatering the residues from the single-stage or multi-stage biomass conversion, preferably to a DS content of >35%, more preferably to a dry substance content of >50% DS and in particular to >60% DS.

32. System according to claim 27, comprising devices suitable for recuperating process water produced in the process and preferably returning it to the process after treatment and/or purification and/or devices suitable for recuperating process heat produced in the process and returning it to the process, wherein the devices for recuperating process heat and/or for heat recirculation preferably comprise components allowing heat exchange which more preferably functions according to the countercurrent principle.

33. System according to claim 27, in which the devices for stabilizing the atmospheric carbon still contained in the conversion residues are preceded by additional devices suitable for pelletizing or briquetting and/or vaporizing, drying, cooling, storing, transporting the conversion residues.

34. System according to claim 27, in which the devices for the single-stage or multi-stage conversion of biomass consist of devices for the anaerobic bacterial fermentation of biomass to biogas and/or bio-methane, which are preferably operated according to the wet fermentation process (wet fermenter), more preferably according to the solid fermentation process (solid fermenter), which are in particular garage fermenters or plug flow fermenters, and in which the at least one garage fermenter is operated with a fermentation cycle of <180 days, preferably with a fermentation cycle of <60 days, more preferably with a fermentation cycle of <35 days, in particular with a fermentation cycle of <21 days and at best with a fermentation cycle of <14 days.

35. System according to claim 29, comprising devices suitable for quenching hot biochar/vegetable coal/biocoke produced by the carbonization devices, preferably with aqueous suspensions selected from slurry, percolate, swill, stillage from ethanol production, liquid residues from anaerobic fermentation, urine, seepage water from silages, process water, processed or purified process water, liquid fermentation mass, permeate, more liquid phase of dehydration, more solid phase of dehydration, any phase of separation, suspensions containing other nutrients and similar suspensions, more preferably with such aqueous suspensions of this selection containing organic nutrients, and in particular process water containing organic nutrients, the organic nutrients of which were previously part of the residues from the single-stage or multi-stage biomass conversion.

36. System according to claim 29, the devices of which for carbonizing conversion residues are suitable for performing both pyrolysis and torrefaction, and/or which comprises devices suitable for loading produced biochar/vegetable coal/biocoke, preferably biochar/vegetable coal/biocoke mixtures and more preferably biochar/vegetable coal/biocoke conversion residue mixtures, with nutrients, for quenching them with water (process water or fresh water), mixing, conveying, warehousing, storing, pelletizing or briquetting them with one another and/or spreading them out on agricultural or forestry land and/or to incorporating them there.

37. System according to claim 27, comprising devices suitable for recuperating, liquefying, purifying, processing, storing, transporting (preferably in liquid aggregate state), delivering to industry, introducing into geological formations, converting into CO₂-based fuel, heating medium or combustion material, performing a combination of these functions, atmospheric carbon dioxide (CO₂) produced in the methods of claims 1 to 26.

38. System according to claim 27, which comprises devices suitable for subjecting the input materials and/or the residues from the single-stage or multi-stage conversion of the biomass into another energy carrier to comminution, preferably chopping or shredding, more preferably a comminution combination consisting of chopping or shredding and grinding, and in particular a comminution combination consisting of bale disintegration, chopping/shredding and grinding, wherein these comminution devices, alone or in combination, are suitable for performing the comminution to an average final particle length of <20 cm, preferably to an average final particle length of <5 cm, more preferably to an average final particle length of <10 mm, in particular to a final particle length of <3 mm and in the best case to a final particle length of <1 mm.

39. System according to claim 27, which comprises devices suitable for subjecting biomass, to a selection of the following treatments before or during the single-stage or multi-stage conversion or residues from the biomass conversion after the single-stage or multi-stage conversion to a selection from the following treatment methods: comminution to a degree of fineness of up to 0.1 mm, soaking/mixing/mashing in cold water or aqueous suspensions, soaking/mixing/mashing in 20° C.-100° C. warm water or aqueous suspensions, biological treatment with fungi, pressurization to >1 bar-500 bar, treatment with >100° C. hot water, treatment with saturated steam, treatment by thermal pressure hydrolysis, steam explosion, treatment by wet oxidation, heating, treatment by extrusion, ultrasonic treatment, treatment by steam reforming, evaporation, sedimentation, crystallization, catalysis, drying, use of polymers, phase separation, particle extraction, combination of a selection of these treatment methods.
